(19) United States
(12) Patent Application Publication
(10) Pub. No.: US 2026/0200905 A1
(43) Pub. Date: Jul. 16, 2026

(54) MICROBIOCIDAL TETRAHYDROISOQUINOLINE DERIVATIVES

(71) Applicant: SYNGENTA CROP PROTECTION AG, Basel (CH)

(72) Inventors: Andrew EDMUNDS, Stein (CH); Christopher Charles SCARBOROUGH, Stein (CH); Atul MAHAJAN, Corlim, Ilhas (IN); Clemens LAMBERTH, Stein (CH); Daniel STIERLI, Stein (CH)

(73) Assignee: SYNGENTA CROP PROTECTION AG, Basel (CH)

(21) Appl. No.: 19/134,122

(22) PCT Filed: Nov. 28, 2023
(86) PCT No.: PCT/EP2023/083420

§ 371 Date: May 29, 2025

(30) Foreign Application Priority Data
Nov. 30, 2022 (IN) 202211069110
Feb. 3, 2023 (EP) 23154887.6

(51) Int. Cl. C07D 413/14; A01N 43/80; A01N 43/82; A01N 43/90; A01P 3/00; C07D 417/14; C07D 471/04

(52) U.S. Cl. CPC C07D 413/14; A01N 43/80; A01N 43/82; A01N 43/90; A01P 3/00; C07D 417/14; C07D 471/04

(57) ABSTRACT

A compound of formula (I) wherein the substituents are as defined in claim 1, and the agrochemically acceptable salts, stereoisomers, enantiomers, tautomers and N-oxides of those compounds, which can be used as fungicides.

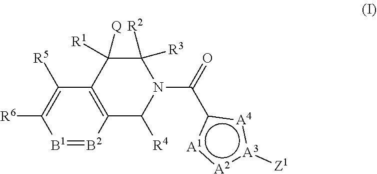

The present invention relates to microbiocidal tetrahydroisoquinoline derivatives, e.g., as active ingredients, which have microbiocidal activity, in particular fungicidal activity. The invention also relates to preparation of these tetrahydroisoquinoline derivatives, to intermediates useful in the preparation of these tetrahydroisoquinoline derivatives, to the preparation of these intermediates, to agrochemical compositions which comprise at least one of the tetrahydroisoquinoline derivatives, to preparation of these compositions and to the use of the tetrahydroisoquinoline derivatives or compositions in agriculture or horticulture for controlling or preventing infestation of plants, harvested food crops, seeds or non-living materials by phytopathogenic microorganisms, in particular fungi.

According to a first aspect of the present invention, there is provided a compound of formula (I)

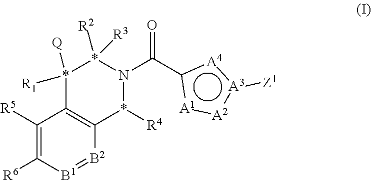

wherein
Q is selected from a 5- or 6-membered heteroaryl, wherein any of said 5- or 6-membered heteroaryl contains 1, 2, 3 or 4 heteroatoms independently selected from N, O or S, with the proviso that no more than one is O or S; and wherein any of said heteroaryl is unsubstituted or substituted with 1, 2 or 3 substituents independently selected from halogen, cyano, C₁-C₄-alkyl, C₁-C₄-haloalkyl, or C₃-C₆-cycloalkyl;
R¹ is selected from hydrogen, halogen, C₁-C₄-haloalkyl, C₃-C₆-cycloalkyl, or C₁-C₄-alkyl;
R² and R³ are independently selected from hydrogen, or C₁-C₄-alkyl;
R⁴ is selected from hydrogen, C₁-C₄-alkyl, C₁-C₄-alkylcarbonyl, N—C₁-C₄-alkoxy-C—C₁-C₄alkyl-carbonimidoyl, N-hydroxy-C—C₁-C₄-alkyl-carbonimidoyl, C₁-C₄-alkoxycarbonyl, N-methoxy-N-methyl-carbonyl, C₁-C₄ alkylaminocarbonyl, di(C₁-C₄ alky)laminocarbonyl, phenyl, a 5- to 6-membered heteroaryl, or C₃-C₈-cycloalkyl; wherein said 5- or 6-membered heteroaryl contains 1, 2, 3 or 4 heteroatoms independently selected from N, O or S, with the proviso that no more than one is O or S; wherein said phenyl and 5- to 6-membered heteroaryl are unsubstituted or substituted with 1, 2 or 3 substituents independently selected from halogen, cyano, C₁-C₄-alkyl, C₁-C₄-haloalkyl, carboxy, or C₁-C₄-alkoxy; and wherein said C₃-C₆-cycloalkyl is unsubstituted or substituted with 1, 2, or 3 substituents independently selected from halogen, cyano, C₁-C₄alkyl, C₁-C₄haloalkyl, or C₁-C₄alkoxy;
B¹ is selected from CR⁷, or N;
B² is selected from CR⁸, or N;
R⁵, R⁶, R⁷ and R⁸ are independently selected from hydrogen, halogen, C₁-C₄-alkyl, C₁-C₄-haloalkyl, C₁-C₄ alkoxy, C₁-C₄-haloalkoxy, C₂-C₄-alkenyloxy, C₂-C₄-alkynyloxy, C₁-C₄-alkylsulfanyl, C₁-C₄-alkylsulfinyl, C₁-C₄ alkylsulfonyl, C₁-C₄-alkoxy-C₁-C₄ alkyl, N—C₁-C₄-alkylamino, N,N-di(C₁-C₄-alkyl)amino, C₁-C₄-alkoxycarbonyl, C₁-C₄ alkylcarbonyl, N—C₁-C₄-alkoxy-C₁-C₄-alkyl-carbonimidoyl, N-hydroxy-C₁-C₄-alkyl-carbonimidoyl, hydroxy, trifluoromethylsulfonyloxy, cyano, carboxy, amino, phenyl, 5- or 6-membered heteroaryl, or C₃-C₆-cycloalkyl, wherein said 5- or 6-membered heteroaryl contains 1, 2, 3 or 4 heteroatoms independently selected from N, O or S, with the proviso that no more than one is O or S; and wherein any of said phenyl, 5- or 6-membered heteroaryl and C₃-C₆-cycloalkyl are unsubstituted or substituted with 1, 2 or 3 substituents independently selected from halogen, cyano, C₁-C₄-alkyl, C₁-C₄ haloalkyl, or C₁-C₄-alkoxy;
A¹, A², A⁴ are independently selected from methine, N, NR¹⁰, O, or S, with the proviso that at least one of A¹, A² and A⁴ is selected from N, O or S, and that no more than one of A¹, A² and A⁴ is O or S, wherein R¹⁰ is selected from hydrogen or C₁-C₄-alkyl
A³ is C or N with the proviso that when A³ is N, A¹, A² and A⁴ are methine, N or NR¹⁰, wherein R¹⁰ is selected from hydrogen or C₁-C₄-alkyl; and
Z¹ is selected from C₁-C₄-alkyl, phenyl, 5- or 6-membered heteroaryl, or C₃-C₆-cycloalkyl, wherein any of said 5- or 6-membered heteroaryl contains 1, 2, 3, or 4 heteroatoms independently selected from N, O, or S, with the proviso that no more than one is O or S; wherein any of said phenyl, and 5- or 6-membered heteroaryl are unsubstituted or substituted by 1, 2, or 3 substituents independently selected from halogen, cyano, C₁-C₄-alkyl, C₂-C₄-alkenyl, C₁-C₄-haloalkyl, C₁-C₄ alkoxy, C₁-C₄-haloalkoxy, C₁-C₄-alkylsulfanyl, C₁-C₄-alkylsulfinyl, C₁-C₄-alkylsulfonyl, or C₂-C₄-alkynyl; and wherein said C₃-C₆-cycloalkyl is unsubstituted or substituted by 1, 2, or 3 substituents independently selected from halogen, cyano, C₁-C₄-alkyl, C₁-C₄-haloalkyl, or C₁-C₄-alkoxy;
or an agrochemically acceptable salt, stereoisomer, or N-oxide thereof, with the proviso that the compound of formula (I) is not

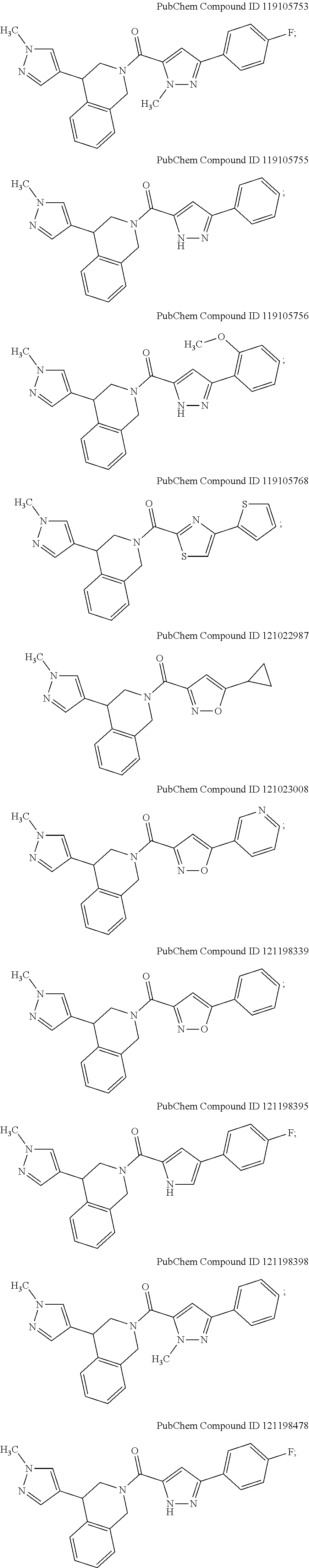

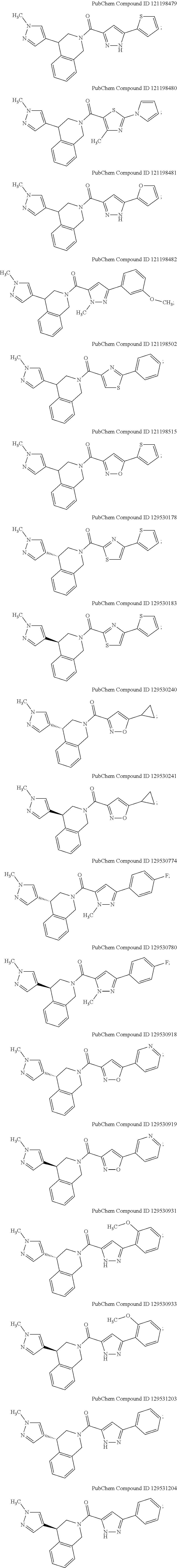

or an agrochemically acceptable salt, stereoisomer, enantiomer, tautomer or N-oxide thereof.

The PubChem Compound ID given for the above disclaimed compounds refers to the identification number of each compound in the PubChem website https://pubchem.ncbi.nlm.nih.gov/.

Surprisingly, it has been found that the compounds of formula (I) have, for practical purposes, a very advantageous level of biological activity for protecting plants against diseases that are caused by fungi.

According to a second aspect of the invention, there is provided an agrochemical composition comprising a fungicidally effective amount of a compound of formula (I) according to the invention. Such an agricultural composition may further comprise at least one additional active ingredient and/or an agrochemically-acceptable diluent or carrier.

According to a third aspect of the invention, there is provided a method of controlling or preventing infestation of useful plants by phytopathogenic microorganisms, wherein a fungicidally effective amount of a compound of formula (I) according to the invention, or a composition comprising the compound of formula (I), is applied to the plants, to parts thereof or the locus thereof.

According to a fourth aspect of the invention, there is provided the use of a compound of formula (I) as a fungicide,

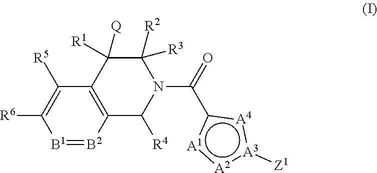

herein
Q is selected from a 5- or 6-membered heteroaryl, wherein any of said 5- or 6-membered heteroaryl contains 1, 2, 3 or 4 heteroatoms independently selected from N, O or S, with the proviso that no more than one is O or S; and wherein any of said heteroaryl is unsubstituted or substituted with 1, 2 or 3 substituents independently selected from halogen, cyano, C₁-C₄-alkyl, C₁-C₄-haloalkyl, or C₃-C₆-cycloalkyl;
R¹ is selected from hydrogen, halogen, C₁-C₄-haloalkyl, C₃-C₆-cycloalkyl, or C₁-C₄-alkyl;
R² and R³ are independently selected from hydrogen, or C₁-C₄-alkyl;
R⁴ is selected from hydrogen, C₁-C₄-alkyl, C₁-C₄-alkylcarbonyl, N—C₁-C₄-alkoxy-C—C₁-C₄alkyl-carbonimidoyl, N-hydroxy-C—C₁-C₄-alkyl-carbonimidoyl, C₁-C₄-alkoxycarbonyl, N-methoxy-N-methyl-carbonyl, C₁-C₄ alkylaminocarbonyl, di(C₁-C₄ alkyl)aminocarbonyl, phenyl, a 5- to 6-membered heteroaryl, or C₃-C₆-cycloalkyl; wherein said 5- or 6-membered heteroaryl contains 1, 2, 3 or 4 heteroatoms independently selected from N, O or S, with the proviso that no more than one is O or S; wherein said phenyl and 5- to 6-membered heteroaryl are unsubstituted or substituted with 1, 2 or 3 substituents independently selected from halogen, cyano, C₁-C₄-alkyl, C₁-C₄-haloalkyl, carboxy, or C₁-C₄-alkoxy; and wherein said C₃-C₆-cycloalkyl is unsubstituted or substituted with 1, 2, or 3 substituents independently selected from halogen, cyano, C₁-C₄alkyl, C₁-C₄haloalkyl, or C₁-C₄alkoxy;
B¹ is selected from CR⁷, or N;
B² is selected from CR⁸, or N;
R⁵, R⁶, R⁷ and R⁸ are independently selected from hydrogen, halogen, C₁-C₄alkyl, C₁-C₄ haloalkyl, C₁-C₄alkoxy, C₁-C₄ haloalkoxy, C₂-C₄alkenyloxy, C₂-C₄ alkynyloxy, C₁-C₄ alkylsulfanyl, C₁-C₄ alkylsulfinyl, C₁-C₄ alkylsulfonyl, C₁-C₄ alkoxy-C₁-C₄ alkyl, N—C₁-C₄alkylamino, N,N-di(C₁-C₄alkyl)amino, C₁-C₄ alkoxycarbonyl, C₁-C₄ alkylcarbonyl, N—C₁-C₄ alkoxy-C₁-C₄ alkyl-carbonimidoyl, N-hydroxy-C₁-C₄ alkyl-carbonimidoyl, hydroxy, trifluoromethylsulfonyloxy, cyano, carboxy, amino, phenyl, 5- or 6-membered heteroaryl, or C₃-C₆ cycloalkyl, wherein said 5- or 6-membered heteroaryl contains 1, 2, 3 or 4 heteroatoms independently selected from N, O or S, with the proviso that no more than one is O or S; and wherein any of said phenyl, 5- or 6-membered heteroaryl and C₃-C₆-cycloalkyl are unsubstituted or substituted with 1, 2 or 3 substituents independently selected from halogen, cyano, C₁-C₄ alkyl, C₁-C₄ haloalkyl, or C₁-C₄alkoxy;
A¹, A², A⁴ are independently selected from methine, N, NR¹⁰, O, or S, with the proviso that at least one of A¹, A² and A⁴ is selected from N, NR¹⁰, O or S, and that no more than one of A¹, A² and A⁴ is O or S, wherein R¹⁰ is selected from hydrogen or C₁-C₄ alkyl;
A³ is C or N with the proviso that when A³ is N, A¹, A² and A⁴ are methine; N or NR¹⁰, wherein R¹⁰ is selected from hydrogen or C₁-C₄alkyl; and
Z¹ is selected from C₁-C₄ alkyl, phenyl, 5- or 6-membered heteroaryl, or C₃-C₆ cycloalkyl, wherein said 5- or 6-membered heteroaryl contains 1, 2, 3, or 4 heteroatoms independently selected from N, O, or S, with the proviso that no more than one is O or S; wherein any of said phenyl, 5- or 6-membered heteroaryl are unsubstituted or substituted by 1, 2, or 3 substituents independently selected from halogen, cyano, C₁-C₄ alkyl, C₂-C₄ alkenyl, C₁-C₄ haloalkyl, C₁-C₄ alkoxy, C₁-C₄ haloalkoxy, C₁-C₄ alkylsulfanyl, C₁-C₄ alkylsulfinyl, C₁-C₄ alkylsulfonyl, or C₂-C₄ alkynyl and wherein said C₃-C₆ cycloalkyl is unsubstituted or substituted by 1, 2, or 3 substituents independently selected from halogen, cyano, C₁-C₄ alkyl, C₁-C₄ haloalkyl, or C₁-C₄alkoxy;
or an agrochemically acceptable salt, stereoisomer, or N-oxide thereof.

According to this particular aspect of the invention, the use may exclude methods for treatment of the human or animal body by surgery or therapy and diagnostic methods practised on the human or animal body.

Compounds of formula (I) which have at least one basic centre can form, for example, acid addition salts, for example with strong inorganic acids such as mineral acids, for example perchloric acid, sulfuric acid, nitric acid, nitrous acid, a phosphorus acid or a hydrohalic acid, with strong organic carboxylic acids, such as C₁-C₄alkanecarboxylic acids which are unsubstituted or substituted, for example by halogen, for example acetic acid, such as saturated or unsaturated dicarboxylic acids, for example oxalic acid, malonic acid, succinic acid, maleic acid, fumaric acid or phthalic acid, such as hydroxycarboxylic acids, for example ascorbic acid, lactic acid, malic acid, tartaric acid or citric acid, or such as benzoic acid, or with organic sulfonic acids, such as C₁-C₄ alkane- or arylsulfonic acids which are unsubstituted or substituted, for example by halogen, for example methane- or p-toluenesulfonic acid. Compounds of formula (I) which have at least one acidic group can form, for example, salts with bases, for example mineral salts such as alkali metal or alkaline earth metal salts, for example sodium, potassium or magnesium salts, or salts with ammonia or an organic amine, such as morpholine, piperidine, pyrrolidine, a mono-, di- or tri-lower-alkylamine, for example ethyl-, diethyl-, triethyl- or dimethylpropylamine, or a mono-, di- or trihydroxy-lower-alkylamine, for example mono-, di- or triethanolamine.

In each case, the compounds of formula (I) according to the invention are in free form, in oxidized form as a N-oxide or in salt form, e.g. an agronomically usable salt form.

N-oxides are oxidized forms of tertiary amines or oxidized forms of nitrogen containing heteroaromatic compounds. They are described for instance in the book “Heterocyclic N-oxides” by A. Albini and S. Pietra, CRC Press, Boca Raton 1991.

The compounds of formula (I) according to the invention also include hydrates which may be formed during the salt formation.

Where substituents are indicated as being “optionally substituted”, this means that they may or may not carry one or more identical or different substituents, e.g., one, two or three R^{x} substituents. For example, C₁-C₆alkyl substituted by 1, 2 or 3 halogens, may include, but not be limited to, —CH₂Cl, —CHCl₂, —CCl₃, —CH₂F, —CHF₂, —CF₃, —CH₂CF₃, or —CF₂CH₃ groups. As another example, C₁-C₆alkoxy substituted by 1, 2, or 3 halogens, may include, but not be limited to, CH₂ClO—, CHCl₂O—, CCl₃O—, CH₂FO—, CHF₂O—, CF₃O—, CF₃CH₂O—, or CH₃CF₂O-groups.

As used herein, the term “halogen” or “halo” refers to fluorine (fluoro), chlorine (chloro), bromine (bromo), or iodine (iodo), preferably fluorine, chlorine, or bromine. This also applies, correspondingly, to halogen in combination with other meanings, such as haloalkyl, haloalkenyl, haloalkynyl, haloalkoxy, and halocycloalkyl.

As used herein, amino means a —NH₂ group.

As used herein, cyano means a —CN group.

As used herein, the term “hydroxyl” or “hydroxy” means an —OH group.

As used herein, the term “carboxylic acid” means a —COOH group.

As used herein, the term “C₁-Cₙ-alkyl” refers to a saturated straight-chain or branched hydrocarbon radical attached via any of the carbon atoms having 1 to n carbon atoms, for example, any one of the radicals methyl, ethyl, n-propyl, 1-methylbutyl, 2-methylbutyl, 3-methylbutyl, 2, 2-dimethylpropyl, 1-ethylpropyl, n-hexyl, n-pentyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, or 1-ethyl-2-methylpropyl.

As used herein, the term “C₂-Cₙ-alkenyl” refers to a straight or branched alkenyl chain moiety having from two to n carbon atoms and one or two double bonds, for example, ethenyl, prop-1-enyl, but-2-enyl.

As used herein, the term “C₂-Cₙ-alkynyl” refers to a straight or branched alkynyl chain moiety having from two to n carbon atoms and one triple bond, for example, ethynyl, prop-2-ynyl, but-3-ynyl, As used herein, the term “C₃-Cₙ-cycloalkyl” refers to three (3) to n membered cycloalkyl radical such as cyclopropyl, cyclobutyl, cyclopentyl and cyclohexyl.

As used herein, the term “C₁-Cₙ-alkoxy” refers to a straight-chain or branched saturated alkyl radical having one (1) to n carbon atoms (as mentioned above) which is attached via an oxygen atom, i.e., for example, any one of the radicals methoxy, ethoxy, n-propoxy, 1-methylethoxy, n-butoxy, 1-methylpropoxy, 2-methylpropoxy and 1,1-dimethylethoxy. The term “C₂-Cₙ-alkenyloxy” as used herein refers to a straight-chain or branched alkenyl chain having two (2) to n carbon atoms (as mentioned above) which is attached via an oxygen atom.

As used herein, the term “C₁-Cₙ-alkoxy-C₁-Cₙ-alkyl” refers to an alkyl radical (as mentioned above) substituted with a C₁-Cₙ-alkoxy group. Examples are methoxymethyl, methoxyethyl, ethoxymethyl and propoxymethyl.

As used herein, the term “C₃-Cₙ-cycloalkyl-C₁-Cₙ-alkyl” refers to an alkyl radical (as mentioned above) substituted with a C₃-Cₙ-cycloalkyl group. Examples are cyclopropylmethyl, cyclopropylethyl. Similarly, the term “C₃-Cₙ-halocycloalkyl-C₁-Cₙ-alkyl” refers to an alkyl radical substituted with cycloalkyl group, wherein the cycloalkyl group is substituted by one or more of the same or different halogen atoms. Examples are 3,3-difluorobutylmethyl and 1-chlorocyclopropylmethyl.

As used herein, the term “C₁-Cₙ-haloalkyl” refers to a straight-chain or branched saturated alkyl radical attached via any of the carbon atoms having 1 to n carbon atoms (as mentioned above), where some or all of the hydrogen atoms in these radicals may be replaced by fluorine, chlorine, bromine and/or iodine, i.e., for example, any one of chloromethyl, dichloromethyl, trichloromethyl, fluoromethyl, difluoromethyl, trifluoromethyl, chlorofluoromethyl, dichlorofluoromethyl, chlorodifluoromethyl, 2-fluoroethyl, 2-chloroethyl, 2-bromoethyl, 2-iodoethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, 2,2,3,3,3-pentafluoropropyl, heptafluoropropyl, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl, or nonafluorobutyl. Accordingly, a term “C₁-C₂fluoroalkyl” would refer to a C₁-C₂alkyl radical which carries 1, 2, 3, 4, or 5 fluorine atoms, for example, any one of difluoromethyl, trifluoromethyl, 1-fluoroethyl, 2-fluoroethyl, 2,2-difluoroethyl, 2,2,2-trifluoroethyl, 1,1,2,2-tetrafluoroethyl, or pentafluoroethyl. Similarly, the term “C₂-Cₙ-haloalkenyl” or “C₂-Cₙ-haloalkynyl” as used herein refers to a C₂-Cₙ-alkenyl or C₂-Cₙ-alkynyl radical respectively substituted with one or more halogen atoms which may be the same or different. Similarly, the term “C₃-Cₙ-halocycloalkyl” or “C₁-Cₙ-haloalkoxy” as used herein refers to a C₃-Cₙ-cycloalkyl radical or C₁-Cₙ-alkoxyl radical respectively substituted with one or more halo atoms which may be the same or different.

As used herein, the term “C₁-Cₙ-alkylthio” or “C₁-Cₙ-alkylsulfanyl” refers to a C₁-Cₙ-alkyl group linked through a sulfur atom.

As used herein, the term “C₁-Cₙ-haloalkylthio” or “C₁-Cₙ-haloalkylsulfanyl” refers to a C₁-Cₙhaloalkyl group linked through a sulfur atom.

As used herein, the term “C₁-Cₙ-alkylsulfinyl” refers to a C₁-Cₙalkyl group linked through the sulfur atom of a sulfinyl (or S(═O)—) group.

As used herein, the term “C₁-Cₙ-alkylsulfonyl” refers to a C₁-Cₙalkyl group linked through the sulfur atom of a sulfonyl (or S(═O)₂—) group.

As used herein, the term “C₁-Cₙ-alkylsulfonyl-C₁-Cₙ-alkyl” refers to an a C₁-Cₙalkyl radical substituted with a C₁-Cₙalkylsulfonyl group.

As used herein, the term “C₁-Cₙ-alkylcarbonyl” refers to a C₁-Cₙ-alkyl group linked through the carbon atom of a carbonyl (C═O) group.

As used herein, the term “C₁-Con-alkoxycarbonyl” refers to a C₁-Cₙ-alkoxy moiety linked through a carbon atom of a carbonyl (or C═O) group.

As used herein, the term “C₁-Cₙ-alkoxycarbonyl-C₁-Cₙ-alkyl” refers to a C₁-Cₙ-alkyl radical substituted by a “C₁-Cₙ-alkoxycarbonyl group.

As used herein, the term “benzoyl” refers to a phenyl group linked through the carbon atom of a carbonyl (C═O) group.

As used herein, the term “C₁-Cₙ-haloalkoxycarbonyl” refers to a C₁-Cₙ-haloalkoxy group linked through the carbon atom of a carbonyl (C═O) group.

As used herein, the term “C₂-Cₙ-alkenyloxycarbonyl” refers to a C₂-Cₙ-alkenyloxycarbonyl group linked through the carbon atom of a carbonyl (C═O) group.

As used herein, the term “C₁-Cₙ-alkylaminocarbonyl” refers to a group of the formula RₐNHC(═O)—, wherein Rₐ is a C₁-Cₙ-alkyl group, as generally defined above, linked through the carbon atom of a carbonyl (C═O) group.

As used herein, the term “di(C₁-Cₙalkyl)aminocarbonyl” refers to a radical of the formula RₐNR_{b}C(═O), where Rₐ is a C₁-Cₙ alkyl radical as generally defined above, and R_{b} is a C₁-Cₙ alkyl radical as generally defined above, linked through the carbon atom of a carbonyl (C═O) group.

As used herein, the term “N—C₁-Cₙ alkylamino” refers to a radical of the formula —NH—Rₐ where Rₐ is a C₁-Cₙ alkyl radical as defined above.

As used herein, the term “N,N-di(C₁-Cₙ alkyl)amino” refers to a radical of the formula —N(Rₐ)Rₐ where each Rₐ is a C₁-Cₙ alkyl radical, which may be the same or different, as defined above.

As used herein, the term “C₁-Cₙ-alkylcarbonyloxy-C₁-Cₙ-alkyl” refers to a C₁-Cₙ-alkyl radical substituted by a C₁-Cₙ-alkylcarbonyloxy (or RₐC(═O)O—) group, wherein Rₐ is a C₁-Cₙ-haloalkyl group.

As used herein, the term “C₁-Cₙ-alkoxycarbonyloxy-C₁-Cₙ-alkyl” refers to a C₁-Cₙ-alkyl radical substituted by a C₁-Cₙ-alkoxycarbonyloxy (or R_{c}C(═O)O—) group, wherein R_{c} is a C₁-Cₙ-alkoxy group. The C₁-Cₙ-alkoxy group linked to the nitrogen may be substituted.

As used herein, the term “N—C₁-C₄ alkoxy-C—C₁-C₄ alkyl-carbonimidoyl” refers to a radical of the formula —C(Rₐ)═NO(R_{b}) where Rₐ is a C₁-C₄ alkyl radical as generally defined above, and R_{b} is a C₁-C₄ alkyl radical as generally defined above.

As used herein the term “N-hydroxy-C—C₁-C₄ alkyl-carbonimidoyl” refers to a radical of the formula —C(Rₐ)═NOH where Rₐ is a C₁-C₄ alkyl radical as generally defined above.

As used herein, the term “aminocarbonyl-C₁-Cₙ-alkyl” refers to a C₁-Cₙ-alkyl radical substituted by an aminocarbonyl (or NH₂C(═O)—) group.

As used herein, the term “C₁-Cₙ-alkylaminocarbonyl-C₁-Cₙ-alkyl” refers to a C₁-Cₙ-alkyl radical substituted by a C₁-Cₙ-alkylaminocarbonyl (or RₐNHC(═O)—) group, wherein Rₐ is a C₁-Cₙ-alkyl group. The C₁-Cₙ-alkyl group linked to the nitrogen may be substituted.

As used herein, the term “heteroaryl” refers to a 5- or 6-membered aromatic monocyclic ring radical which contains 1, 2, 3 or 4 heteroatoms independently selected from N, O or S. Examples of heteroaryl include, but are not limited to, furanyl, pyrrolyl, thienyl, pyrazolyl, imidazolyl, thiazolyl, isothiazolyl, oxazolyl, isoxazolyl, triazolyl, tetrazolyl, pyrazinyl, pyridazinyl, pyrimidyl or pyridyl. The term “heteroaryl-C₁-Cₙ-alkyl” or “heteroaryl-C₃-Cₙ-cycloalkyl” refers to an C₁-Cₙ-alkyl or C₃-Cₙ-cycloalkyl radical respectively substituted by a heteroaryl group. The heteroaryl-C₁-Cₙ-alkyl or heteroaryl-C₃-Cₙ-cycloalkyl radical may be substituted on heteroaryl, alkyl and/or cycloalkyl group as appropriate.

As used herein, the term “controlling” refers to reducing the number of pests, eliminating pests and/or preventing further pest damage such that damage to a plant or to a plant derived product is reduced.

As used herein, the term “pest” refers to insects, and molluscs that are found in agriculture, horticulture, forestry, the storage of products of vegetable origin (such as fruit, grain and timber); and those pests associated with the damage of man-made structures. The term pest encompasses all stages in the life cycle of the pest.

As used herein, the term “effective amount” refers to the amount of the compound, or a salt thereof, which, upon single or multiple applications provides the desired effect.

An effective amount is readily determined by the skilled person in the art, using known techniques and by observing results obtained under analogous circumstances. In determining the effective amount, a number of factors are considered including, but not limited to the type of plant or derived product to be applied; the pest to be controlled & its lifecycle; the particular compound applied; the type of application; and other relevant circumstances.

As used herein, the term “room temperature” or “RT” or “rt” refer to a temperature of about 15° C. to about 35° C. For example, rt can refer to a temperature of about 20° C. to about 30° C.

The following list provides definitions, including preferred definitions, for substituents R¹, R², R³, R⁴, R⁵, R⁶, R⁷, R⁸, B¹, B², A¹, A², A³, A⁴, Q and Z with reference to the compounds of formula (I) of the present invention. For any one of these substituents, any of the definitions given below may be combined with any definition of any other substituent given below or elsewhere in this document.

In one embodiment of the invention, R¹ is selected from hydrogen, halogen, C₁-C₄ alkyl, C₁-C₄ haloalkyl, or C₃-C₆ cycloalkyl. Preferably R¹ is hydrogen, chlorine, bromine, fluorine, methyl, ethyl, trifluoromethyl, difluoromethyl, or cyclopropyl. More preferably R¹ is hydrogen, chlorine, bromine, or methyl. Still more preferably R¹ is hydrogen, or methyl. Most preferably R¹ is hydrogen. In one embodiment of the invention R¹ is hydrogen. In another embodiment of the invention R¹ is methyl,

In one embodiment of the invention, R², R³ are independently selected from hydrogen, or C₁-C₄-alkyl. Preferably R², R³ are independently selected from hydrogen, methyl, or ethyl. More preferably, R², R³ are independently selected from hydrogen, or methyl. Even more preferably, R² and R³ are hydrogen.

In one embodiment of the invention, R⁴ is selected from hydrogen, C₁-C₄-alkyl, C₁-C₄-alkylcarbonyl, N—C₁-C₄-alkoxy-C—C₁-C₄ alkyl-carbonimidoyl, N-hydroxy-C—C₁-C₄-alkyl-carbonimidoyl, C₁-C₄-alkoxycarbonyl, N-methoxy-N-methyl-carbonyl, C₁-C₄ alkylaminocarbonyl, di(C₁-C₄ alkyl)aminocarbonyl, phenyl, a 5- to 6-membered heteroaryl, or C₃-C₆-cycloalkyl; wherein said 5- or 6-membered heteroaryl contains 1, 2, 3 or 4 heteroatoms independently selected from N, O or S, with the proviso that no more than one is O or S; wherein said phenyl and 5- to 6-membered heteroaryl are unsubstituted or substituted with 1, 2 or 3 substituents independently selected from halogen, cyano, C₁-C₄-alkyl, C₁-C₄-haloalkyl, carboxy, or C₁-C₄-alkoxy; and wherein said C₃-C₆-cycloalkyl is unsubstituted or substituted with 1, 2, or 3 substituents independently selected from halogen, cyano, C₁-C₄alkyl, C₁-C₄haloalkyl, or C₁-C₄alkoxy.

In another embodiment of the invention R⁴ is selected from hydrogen, C₁-C₄alkyl, C₃-C₁-C₄alkylcarbonyl, C₁-C₄alkoxycarbonyl, N—C₁-C₄ alkoxy-C—C₁-C₄alkyl-carbonimidoyl, N-hydroxy-C—C₁-C₄ alkyl-carbonimidoyl, N-methoxy-N-methyl-carbonyl, C₁-C₄ alkylaminocarbonyl, di(C₁-C₄ alkyl)aminocarbonyl, phenyl, a 5- to 6-membered heteroaryl, or C₃-C₆-cycloalkyl, wherein said 5- to 6-membered heteroaryl contains 1 heteroatom selected from N. and wherein said phenyl and 5- to 6-membered heteroaryl are unsubstituted or substituted with 1 to 2 substituent independently selected from halogen, C₁-C₄haloalkyl, cyano, or C₁-C₄alkyl; and wherein said C₃-C₆-cycloalkyl is unsubstituted or substituted with 1 substituent selected from cyano. Preferably R⁴ is selected from hydrogen, methyl, acetyl, C(CH₃)═NOCH₃, —C(CH₃)═NOCH₂CH₃, —C(CH₃)═NOH, methoxycarbonyl, ethoxycarbonyl, N-methoxy-N-methyl-carbonyl, methylaminocarbonyl, dimethylaminocarbonyl, phenyl, 2-cyanophenyl, 3-cyanophenyl, 4-cyanophenyl, [4-(trifluoromethyl)pyrazol-1-yl], [3-(trifluoromethyl)pyrazol-1-yl], 3-cyanopyrazol-1-yl, 4-cyanopyrazol-1-yl, 5-chloropyrazol-1-yl, 5-fluoropyrazol-1-yl, 3,5-dimethylpyrazol-1-yl, 5-methylpyrazol-1-yl, pyrazol-1-yl, cyclopropyl, or 1-cyanocyclopropyl. More preferably R⁴ is selected from hydrogen, methyl, acetyl, C(CH₃)═NOCH₃, —C(CH₃)═NOCH₂CH₃, —C(CH₃)═NOH, phenyl, 4-cyanophenyl, pyrazol-1-yl, cyclopropyl, or 1-cyanocyclopropyl. Even more preferably R⁴ is selected from hydrogen, methyl, cyclopropyl, or 1-cyanocyclopropyl.

In another embodiment R⁴ is selected from hydrogen, C₁-C₄ alkyl, or C₃-C₆-cycloalkyl. Preferably R⁴ is hydrogen, methyl, ethyl, cyclopropyl, or 1-cyanocyclopropyl. Even more preferably R⁴ is hydrogen, methyl, ethyl, or cyclopropyl.

In another preferred embodiment R⁴ is hydrogen, or C₁-C₄ alkyl. Preferably R⁴ is hydrogen, methyl, or ethyl.

More preferably R⁴ is hydrogen, or methyl.

In an embodiment of the invention, B¹ is CR⁷ and B² is CR⁸, or B¹ is N and B² is CR⁸, or B¹ is CR⁷ and B² is N. Preferably, B¹ is CR⁷ and B² is CR⁷.

In one embodiment of the invention R⁵ and R⁶ are independently selected from hydrogen, halogen, C₁-C₄alkyl, C₁-C₄ haloalkyl, C₁-C₄ alkoxy, C₁-C₄ haloalkoxy, C₂-C₄alkenyloxy, C₂-C₄ alkynyloxy, C₁-C₄ alkylsulfanyl, C₁-C₄ alkylsulfinyl, C₁-C₄ alkylsulfonyl, C₁-C₄ alkoxy-C₁-C₄ alkyl, N—C₁-C₄alkylamino, N,N-di(C₁-C₄alkyl)amino, C₁-C₄ alkoxycarbonyl, C₁-C₄ alkylcarbonyl, N—C₁-C₄ alkoxy-C₁-C₄ alkyl-carbonimidoyl, N-hydroxy-C₁-C₄ alkyl-carbonimidoyl, hydroxy, trifluoromethylsulfonyloxy, cyano, carboxy, amino, phenyl, 5- or 6-membered heteroaryl, or C₃-C₆ cycloalkyl, wherein said 5- or 6-membered heteroaryl contains 1, 2, 3 or 4 heteroatoms independently selected from N, O or S, with the proviso that no more than one is O or S; and wherein any of said phenyl, 5- or 6-membered heteroaryl and C₃-C₆-cycloalkyl are unsubstituted or substituted with 1, 2 or 3 substituents independently selected from halogen, cyano, C₁-C₄ alkyl, C₁-C₄ haloalkyl, or C₁-C₄alkoxy.

In one embodiment of the invention, R⁵ and R⁶ are independently selected from hydrogen, halogen, cyano, C₁-C₄ alkyl, or C₁-C₄ alkoxy. Preferably, R⁵ and R⁶ are independently selected from hydrogen, halogen, methyl, methoxy, or cyano. More preferably, R⁵ and R⁶ are independently selected from hydrogen, methyl, chlorine, fluorine, bromine, or methoxy. Even more preferably, R⁵ and R⁶ are independently selected from hydrogen, or methoxy.

In another embodiment of the invention, R⁵ is hydrogen, halogen, or cyano. Preferably R⁵ is hydrogen, bromine, chlorine, or cyano. More preferably, R⁵ is hydrogen, cyano, or bromine. Even more preferably, R⁵ is hydrogen.

In another embodiment R⁶ is selected from hydrogen, halogen, C₁-C₃ alkyl, C₁-C₂ haloalkyl, C₁-C₃ haloalkoxy, C₁-C₄ alkoxy, C₁-C₃alkenyloxy, C₁-C₃alkynyloxy, C₁-C₂alkylsulfanyl, C₁-C₂ alkylsulfinyl, C₁-C₂ alkylsulfonyl, C₁-C₂ alkoxy-C₁-C₂ alkyl, C₁-C₃alkoxycarbonyl, C₁-C₂alkylcarbonyl, N—C₁-C₂ alkoxy-C—C₁-C₂ alkyl-carbonimidoyl, N-hydroxy-C—C₁-C₂alkyl-carbonimidoyl hydroxy, C₁-C₂alkylaminocarbonyl, di(C₁-C₂alkyl)aminocarbonyl, trifluoromethylsulfonyloxy, cyano, carboxy, phenyl, 2-cyanophenyl, 3-cyanophenyl, 4-cyanophenyl, 2-methylphenyl, 3-methylphenyl, 4-methylphenyl, [4-(trifluoromethyl)pyrazol-1-yl], [3-(trifluoromethyl)pyrazol-1-yl], 3-cyanopyrazol-1-yl, 4-cyanopyrazol-1-yl, 5-chloropyrazol-1-yl, 4-chloropyrazol-1-yl, 3-chloropyrazol-1-yl, 5-fluoropyrazol-1-yl, 4-fluoropyrazol-1-yl, 3-fluoropyrazol-1-yl, 3,5-dimethylpyrazol-1-yl, 5-methylpyrazol-1-yl, 4-methylpyrazol-1-yl, 3-methylpyrazol-1-yl, pyrazol-1-yl, cyclopropyl, or 1-cyanocyclopropyl. Preferably R⁶ is hydrogen, chloro, fluoro, bromo, methyl, ethyl, trifluoromethyl, difluoromethyl, difluoromethoxy, 2,2-difluoroethoxy, 2,2,2-trifluoroethoxy, methoxy, ethoxy, propoxy, allyloxy, prop-2-ynoxy, methylsulfanyl, methylsulfinyl, methylsulfonyl, methoxymethyl, ethoxymethyl, 2-methoxyethoxymethyl, methoxycarbonyl, ethoxycarbonyl, tert-butoxycarbonyl, acetyl, propanoyl, —C(CH₃)═NOCH₃, —C(CH₃)═NOCH₂CH₃, —C(CH₃)=NOH, methylaminocarbonyl, di(methylamino)carbonyl, trifluoromethylsulfonyloxy, cyano, carboxy, phenyl, 2-cyanophenyl, 3-cyanophenyl, 4-cyanophenyl, 2-methylphenyl, 3-methylphenyl, 4-methylphenyl, [4-(trifluoromethyl)pyrazol-1-yl], [3-(trifluoromethyl)pyrazol-1-yl], 3-cyanopyrazol-1-yl, 4-cyanopyrazol-1-yl, 5-chloropyrazol-1-yl, 4-chloropyrazol-1-yl, 3-chloropyrazol-1-yl, 5-fluoropyrazol-1-yl, 4-fluoropyrazol-1-yl, 3-fluoropyrazol-1-yl, 3,5-dimethylpyrazol-1-yl, 5-methylpyrazol-1-yl, 4-methylpyrazol-1-yl, 3-methylpyrazol-1-yl, pyrazol-1-yl, cyclopropyl, or 1-cyanocyclopropyl. More preferably R⁶ is hydrogen, chloro, bromo, fluoro, cano, methyl, methoxy, propoxy, allyloxy, methoxymethyl, 2-methoxyethoxymethyl, phenyl, 2-cyanophenyl, 3-cyanophenyl, 4-cyanophenyl, [4-(trifluoromethyl)pyrazol-1-yl], [3-(trifluoromethyl)pyrazol-1-yl], 3-cyanopyrazol-1-yl, 4-cyanopyrazol-1-yl, 5-chloropyrazol-1-yl, 4-chloropyrazol-1-yl, 3-chloropyrazol-1-yl, 5-fluoropyrazol-1-yl, 4-fluoropyrazol-1-yl, 3-fluoropyrazol-1-yl, 3,5-dimethylpyrazol-1-yl, 5-methylpyrazol-1-yl, 4-methylpyrazol-1-yl, 3-methylpyrazol-1-yl, pyrazol-1-yl, cyclopropyl, or 1-cyanocyclopropyl. Even more preferably R⁶ is hydrogen, chloro, bromo, cyano, methyl, or methoxy. Most preferably R⁶ is hydrogen, or methoxy.

In one embodiment of the invention R⁷ and R⁸ are independently selected from hydrogen, halogen, C₁-C₄alkyl, C₁-C₄ haloalkyl, C₁-C₄ alkoxy, C₁-C₄ haloalkoxy, C₂-C₄alkenyloxy, C₂-C₄ alkynyloxy, C₁-C₄ alkylsulfanyl, C₁-C₄ alkylsulfinyl, C₁-C₄ alkylsulfonyl, C₁-C₄ alkoxy-C₁-C₄ alkyl, N—C₁-C₄alkylamino, N,N-di(C₁-C₄alkyl)amino, C₁-C₄ alkoxycarbonyl, C₁-C₄ alkylcarbonyl, N—C₁-C₄ alkoxy-C₁-C₄ alkyl-carbonimidoyl, N-hydroxy-C₁-C₄ alkyl-carbonimidoyl, hydroxy, trifluoromethylsulfonyloxy, cyano, carboxy, amino, phenyl, 5- or 6-membered heteroaryl, or C₃-C₆ cycloalkyl, wherein said 5- or 6-membered heteroaryl contains 1, 2, 3 or 4 heteroatoms independently selected from N, O or S, with the proviso that no more than one is O or S; wherein any of said phenyl, 5- or 6-membered heteroaryl and C₃-C₆-cycloalkyl are unsubstituted or substituted with 1, 2 or 3 substituents independently selected from halogen, cyano, C₁-C₄ alkyl, C₁-C₄ haloalkyl, or C₁-C₄alkoxy. Preferably R⁷ and R⁸ are independently selected from hydrogen, chloro, fluoro, bromo, methyl, ethyl, trifluoromethyl, difluoromethyl, difluoromethoxy, 2,2-difluoroethoxy, 2,2,2-trifluoroethoxy, methoxy, ethoxy, propoxy allyloxy, prop-2-ynoxy, methylsulfanyl, methylsulfinyl, methylsulfonyl, methoxymethyl, ethoxymethyl, 2-methoxyethoxymethyl, methoxycarbonyl, ethoxycarbonyl, tert-butoxycarbonyl, acetyl, propanoyl, —C(CH₃)=NOCH₃, —C(CH₃)=NOCH₂CH₃, —C(CH₃)=NOH, methylaminocarbonyl, di(methylamino)carbonyl, trifluoromethylsulfonyloxy, cyano, carboxy, phenyl, 2-cyanophenyl, 3-cyanophenyl, 4-cyanophenyl, 2-methylphenyl, 3-methylphenyl, 4-methylphenyl, [4-(trifluoromethyl)pyrazol-1-yl], [3-(trifluoromethyl)pyrazol-1-yl], 3-cyanopyrazol-1-yl, 4-cyanopyrazol-1-yl, 5-chloropyrazol-1-yl, 4-chloropyrazol-1-yl, 3-chloropyrazol-1-yl, 5-fluoropyrazol-1-yl, 4-fluoropyrazol-1-yl, 3-fluoropyrazol-1-yl, 3,5-dimethylpyrazol-1-yl, 5-methylpyrazol-1-yl, 4-methylpyrazol-1-yl, 3-methylpyrazol-1-yl, pyrazol-1-yl, cyclopropyl, or 1-cyanocyclopropyl. More preferably R⁷ and R⁸ are independently selected from hydrogen, chloro, bromo, fluoro, cyano, methyl, methoxy, propoxy, allyloxy, methoxymethyl, 2-methoxyethoxymethyl, phenyl, 2-cyanophenyl, 3-cyanophenyl, 4-cyanophenyl, [4-(trifluoromethyl)pyrazol-1-yl], [3-(trifluoromethyl)pyrazol-1-yl], 3-cyanopyrazol-1-yl, 4-cyanopyrazol-1-yl, 5-chloropyrazol-1-yl, 4-chloropyrazol-1-yl, 3-chloropyrazol-1-yl, 5-fluoropyrazol-1-yl, 4-fluoropyrazol-1-yl, 3-fluoropyrazol-1-yl, 3,5-dimethylpyrazol-1-yl, 5-methylpyrazol-1-yl, 4-methylpyrazol-1-yl, 3-methylpyrazol-1-yl, pyrazol-1-yl, C(CH₃)=NOCH₃, —C(CH₃)=NOCH₂CH₃, —C(CH₃)=NOH, cyclopropyl, or 1-cyanocyclopropyl. Still more preferably R⁷ and R⁸ are independently selected from hydrogen, chlorine, bromine, or cyano.

In another embodiment R⁷ and R⁸ are independently selected from hydrogen, halogen, C₁-C₃ alkyl, or C₁-C₄ alkoxy. Preferably R⁷ and R⁸ are independently selected from hydrogen, or halogen. More preferably R⁷ and R⁸ are hydrogen, bromine, or chlorine. Even more preferably R⁷ and R³ are hydrogen.

In one embodiment of the invention A¹, A², A⁴ are independently selected from methine, N, O, or S, with the proviso that at least one of A¹, A² and A⁴ is selected from N, O or S, and that no more than one of A¹, A² or A⁴ is O or S.

In one embodiment of the invention A³ is C or N, with the proviso that when A³ is N, A¹, A² and A⁴ are methine or N.

In one embodiment of the invention, Z¹ is selected from C₁-C₄ alkyl, phenyl, 5- or 6-membered heteroaryl, or C₃-C₆ cycloalkyl, wherein said 5- or 6-membered heteroaryl contains 1, 2, 3, or 4 heteroatoms independently selected from N, O, or S, with the proviso that no more than one is O or S; wherein any of said phenyl, 5- or 6-membered heteroaryl are unsubstituted or substituted by 1, 2, or 3 substituents independently selected from halogen, cyano, C₁-C₄ alkyl, C₂-C₄ alkenyl, C₁-C₄ haloalkyl, C₁-C₄ alkoxy, C₁-C₄ haloalkoxy, C₁-C₄ alkylsulfanyl, C₁-C₄ alkylsulfinyl, C₁-C₄ alkylsulfonyl, or C₂-C₄ alkynyl; and wherein said C₃-C₆ cycloalkyl is unsubstituted or substituted by 1, 2, or 3 substituents independently selected from halogen, cyano, C₁-C₄ alkyl, C₁-C₄ haloalkyl, or C₁-C₄alkoxy.

In another embodiment Z¹ is selected from C₁-C₄ alkyl, phenyl, a 5- to 6-membered heteroaryl, or C₃-C₆-cycloalkyl; wherein said 5- or 6-membered heteroaryl contains 1, 2, or 3 heteroatoms independently selected from N, O, or S, with the proviso that no more than one is O or S; wherein any of said phenyl and 5- to 6-membered heteroaryl are unsubstituted or substituted with 1, 2, or 3 substituents independently selected from fluoro, chloro, methyl, ethyl, ethynyl, methoxy, or methylsulfonyl; and wherein said C₃-C₆-cycloalkyl is unsubstituted or substituted with 1 or 2 substituents selected from methyl.

In another embodiment Z¹ is selected from C₁-C₄ alkyl, phenyl, a 5- to 6-membered heteroaryl, or C₃-C₆-cycloalkyl, wherein said 5- or 6-membered heteroaryl contains 1 heteroatom selected from N, O, or S, wherein any of said phenyl and 5- to 6-membered heteroaryl are unsubstituted or substituted with 1 or 2 substituents independently selected from fluoro, chloro, methyl, ethyl, ethynyl, methoxy, or methylsulfonyl; and wherein said C₃-C₆-cycloalkyl is unsubstituted or substituted with 1 or 2 substituents selected from methyl.

In still another embodiment Z¹ is selected from C₁-C₄ alkyl, phenyl, a 5- to 6-membered heteroaryl, or C₃-C₆-cycloalkyl, wherein said 5- or 6-membered heteroaryl contains 1 heteroatom selected from N; wherein any of said phenyl and 5- to 6-membered heteroaryl are unsubstituted or substituted with 1 or 2 substituents independently selected from fluoro, chloro, methyl, ethyl, ethynyl, methoxy, or methylsulfonyl; and wherein said C₃-C₆-cycloalkyl is unsubstituted or substituted with 1 or 2 substituents selected from methyl.

Preferably Z¹ is selected from 1-methylpyrazol-4-yl, 2,3,4-trifluorophenyl, 2,3-difluorophenyl, 3,4-difluorophenyl, 2,4,6-trifluorophenyl, 2,4-difluorophenyl, 2,5-difluorophenyl, 3,5-difluoro-2-pyridyl, 5-fluoro-2-pyridyl, 3-fluoro-2-pyridyl, 2-fluoro-4-methoxy-phenyl, 2-fluoro-4-methylsulfonyl-phenyl, 2-fluorophenyl, 3-fluorophenyl, 4-fluorophenyl, 3,5-difluoro-2-furyl, 3-fluoro-2-furyl, 5-fluoro-2-furyl, 3,5-difluoro-2-thienyl, 3-fluoro-2-thienyl, 5-fluoro-2-thienyl, 2-methylphenyl, 3-methylphenyl, 4-methylphenyl, 2-chlorophenyl, 3-chlorophenyl, 4-chlorophenyl, 3-methoxyphenyl, 4-ethynyl-2-fluoro-phenyl, 4-fluoro-2-methoxy-phenyl, cyclopropyl, 1-methylcyclopropyl, cyclobutyl, cyclohexyl, cyclopentyl, methyl, n-propyl, or phenyl. More preferably Z¹ is selected from 1-methylpyrazol-4-yl, 2,4,6-trifluorophenyl, 2,4-difluorophenyl, 2,5-difluorophenyl, 2-chlorophenyl, 2-fluorophenyl, 3,5-difluoro-2-pyridyl, 2-furyl, 2-methylphenyl, 2-thienyl, 3,4-difluorophenyl, 3-chlorophenyl, 3-fluorophenyl, 3-methylphenyl, 3-thienyl, 4-fluoro-2-methoxy-phenyl, 4-fluorophenyl, 4-methylphenyl, cyclobutyl, cyclohexyl, cyclopentyl, methyl, or phenyl. Even more preferably Z¹ is selected from 1-methylpyrazol-4-yl, 2,4,6-trifluorophenyl, 3,5-difluoro-2-pyridyl, 2,4-difluorophenyl, 2-fluorophenyl, 2-furyl, 2-methylphenyl, 2-thienyl, 3,4-difluorophenyl, 3-chlorophenyl, 3-thienyl, 4-fluoro-2-methoxy-phenyl, 4-fluorophenyl, cyclobutyl, cyclohexyl, cyclopentyl, or phenyl. Still even more preferably Z¹ is selected from 2,4-difluorophenyl, 3,5-difluoro-2-pyridyl, 2-fluorophenyl, 4-fluorophenyl, or phenyl.

In another embodiment of the invention Z¹ is selected from C₁-C₄ alkyl, phenyl, a 5- to 6-membered heteroaryl, or C₃-C₆-cycloalkyl; wherein said 5- or 6-membered heteroaryl contains 1, 2 or 3 heteroatoms independently selected from N, O, or S, with the proviso that no more than one is O or S; wherein said phenyl and 5- to 6-membered heteroaryl are unsubstituted or substituted with 1 or 2 substituents independently selected from halogen, cyano, C₁-C₄ alkyl, C₁-C₄ haloalkyl, C₁-C₄ haloalkoxy, or C₁-C₄ alkoxy; and wherein said C₃-C₅-cycloalkyl is unsubstituted or substituted with 1 substituent selected from halogen or C₁-C₄ alkyl. Preferably Z¹ is selected from phenyl or a 5- to 6-membered heteroaryl; wherein said 5- or 6-membered heteroaryl contains 1 heteroatom selected from N or S; and wherein said phenyl and -5 to 6-membered heteroaryl are unsubstituted or substituted with 1 or 2 substituents independently selected from fluoro, chloro, C₁-C₄alkyl, or C₁-C₄alkoxy. More preferably Z¹ is selected from 1-methylpyrazol-4-yl, 2,4,6-trifluorophenyl, 2,4-difluorophenyl, 3,5-difluoro-2-pyridyl, 5-fluoro-2-pyridyl, 3-fluoro-2-pyridyl, 2-fluorophenyl, 3,5-difluoro-2-furyl, 3-fluoro-2-furyl, 5-fluoro-2-furyl, 3,5-difluoro-2-thienyl, 3-fluoro-2-thienyl, 5-fluoro-2-thienyl, 2-methylphenyl, 2-thienyl, 3,4-difluorophenyl, 3-chlorophenyl, 3-thienyl, 4-fluoro-2-methoxy-phenyl, 4-fluorophenyl, cyclobutyl, cyclohexyl, cyclopentyl, or methyl. Still more preferably Z¹ is selected from 2,4-difluorophenyl, 3,5-difluoro-2-pyridyl, 5-fluoro-2-pyridyl, 3-fluoro-2-pyridyl, 3,5-difluoro-2-furyl, 3-fluoro-2-furyl, 5-fluoro-2-furyl, 3,5-difluoro-2-thienyl, 3-fluoro-2-thienyl, 5-fluoro-2-thienyl, 2-fluorophenyl, 4-fluorophenyl, or phenyl.

In another embodiment of the invention Z¹ is selected from phenyl, a 5- to 6-membered heteroaryl, or C₃-C₆-cycloalkyl; wherein said 5- or 6-membered heteroaryl contains 1 heteroatom selected from N or S, and wherein said phenyl and 5- to 6-membered heteroaryl are unsubstituted or substituted with 1 or 2 substituents independently selected from halogen, cyano, C₁-C₄ alkyl, C₁-C₄ haloalkyl, C₁-C₄ haloalkoxy, or C₁-C₄ alkoxy and wherein said C₃-C₆-cycloalkyl is unsubstituted or substituted with 1 substituent selected from halogen, or C₁-C₄ alkyl. Preferably Z¹ is selected from phenyl, or a 5- to 6-membered heteroaryl; wherein said 5- or 6-membered heteroaryl contains 1 heteroatom selected from N or S, and wherein said phenyl and 5- to 6-membered heteroaryl are unsubstituted or substituted with 1 or 2 substituents independently selected from fluoro, chloro, C₁-C₄ alkyl, or C₁-C₄ alkoxy.

In a further embodiment of the invention, Z¹ is selected from phenyl or a 5- to 6-membered heteroaryl; wherein said 5- or 6-membered heteroaryl contains 1 heteroatom selected from N, and wherein said phenyl and 5- to 6-membered heteroaryl are unsubstituted or substituted with 1 or 2 substituents independently selected from fluoro, chloro, C₁-C₄alkyl, or C₁-C₄alkoxy. Preferably Z¹ is selected from 1-methylpyrazol-4-yl, 2,3,4-trifluorophenyl, 2,3-difluorophenyl, 3,4-difluorophenyl, 2,4,6-trifluorophenyl, 2,4-difluorophenyl, 2,5-difluorophenyl, 3,5-difluoro-2-pyridyl, 5-fluoro-2-pyridyl, 3-fluoro-2-pyridyl, 2-fluoro-4-methoxy-phenyl, 2-fluoro-4-methylsulfonyl-phenyl, 2-fluorophenyl, 3-fluorophenyl, 4-fluorophenyl, 3,5-difluoro-2-furyl, 3-fluoro-2-furyl, 5-fluoro-2-furyl, 3,5-difluoro-2-thienyl, 3-fluoro-2-thienyl, 5-fluoro-2-thienyl, 2-methylphenyl, 3-methylphenyl, 4-methylphenyl, 2-chlorophenyl, 3-chlorophenyl, 4-chlorophenyl, 3-methoxyphenyl, 4-ethynyl-2-fluoro-phenyl, 4-fluoro-2-methoxy-phenyl, cyclopropyl, 1-methylcyclopropyl, cyclobutyl, cyclohexyl, cyclopentyl, methyl, n-propyl or phenyl. More preferably Z¹ is selected from 1-methylpyrazol-4-yl, 2,4,6-trifluorophenyl, 2,4-difluorophenyl, 2,5-difluorophenyl, 2-chlorophenyl, 2-fluorophenyl, 2-furyl, 2-methylphenyl, 2-thienyl, 3,4-difluorophenyl, 3-chlorophenyl, 3-fluorophenyl, 3-methylphenyl, 3-thienyl, 4-fluoro-2-methoxy-phenyl, 4-fluorophenyl, 4-methylphenyl, cyclobutyl, cyclohexyl, cyclopentyl, methyl or phenyl. Even more preferably Z¹ is selected from 1-methylpyrazol-4-yl, 2,4,6-trifluorophenyl, 3,5-difluoro-2-pyridyl, 2,4-difluorophenyl, 2-fluorophenyl, 2-furyl, 2-methylphenyl, 2-thienyl, 3,4-difluorophenyl, 3-chlorophenyl, 3-thienyl, 4-fluoro-2-methoxy-phenyl, 4-fluorophenyl, cyclobutyl, cyclohexyl, cyclopentyl or phenyl. Still even more preferably Z¹ is selected from 2,4-difluorophenyl, 3,5-difluoro-2-pyridyl, 2-fluorophenyl, 4-fluorophenyl or phenyl.

In one embodiment of the invention, Q is selected from a 5- or 6-membered heteroaryl, wherein said 5- or 6-membered heteroaryl contains 1, 2, 3 or 4 heteroatoms independently selected from N, O or S, with the proviso that no more than one is O or S; and wherein said heteroaryl is unsubstituted or substituted with 1, 2 or 3 substituents independently selected from halogen, cyano, C₁-C₄-alkyl, C₁-C₄-haloalkyl, or C₃-C₆cycloalkyl.

In another embodiment of the invention Q is selected from Q1, Q2, Q3, Q4, Q5, Q6, Q7, Q8, Q9, Q10, Q11, Q12, Q13, Q14, Q15, or Q16,

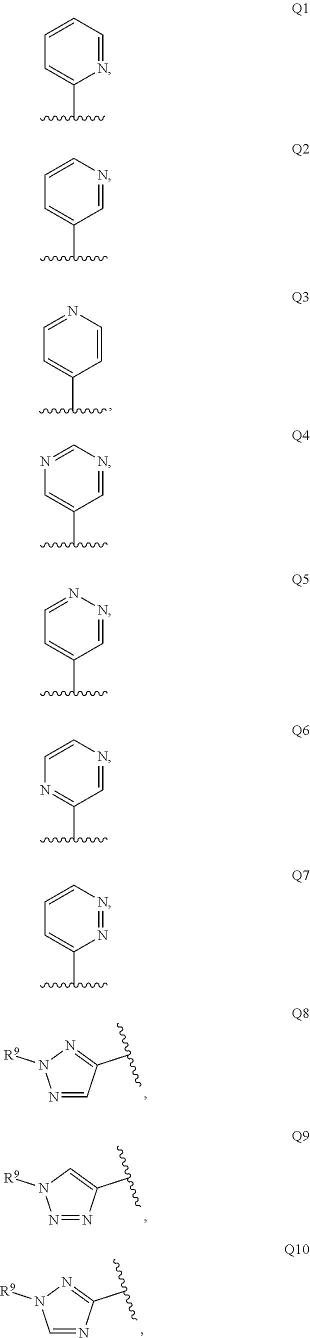

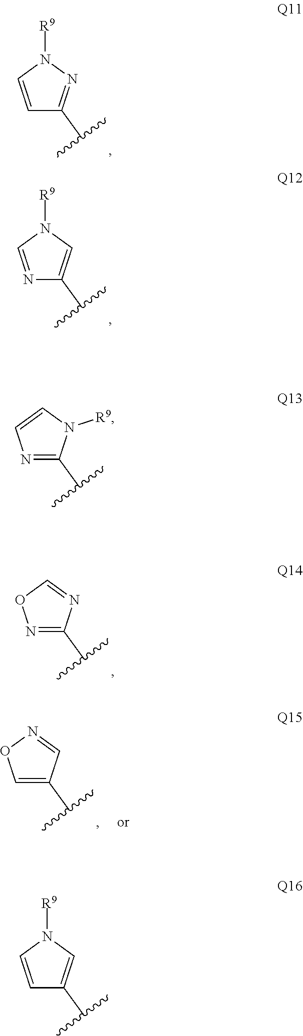

and wherein any of said Q1 to Q16 are unsubstituted or substituted with 1, 2 or 3 substituents selected from C₁-C₄ alkyl, halogen, C₁-C₄-haloalkyl, or C₃-C₆cycloalkyl, and wherein R⁹ is selected from hydrogen or C₁-C₄ alkyl, wherein [symbol] denotes the position, which is attached to the tetrahydroisoquinoline-moiety.

In a preferred embodiment of the invention, Q is selected from Q1, Q2, Q3, Q4, Q6, Q7, Q9, Q10, Q11, Q12, Q14, or Q16,

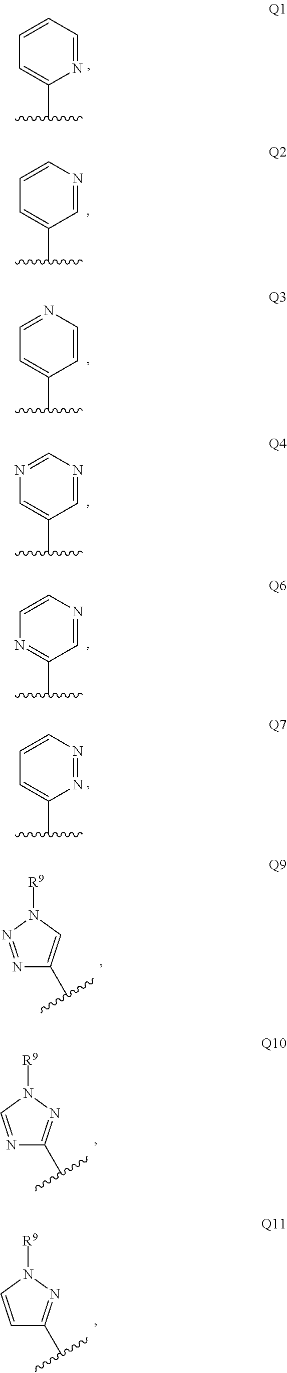

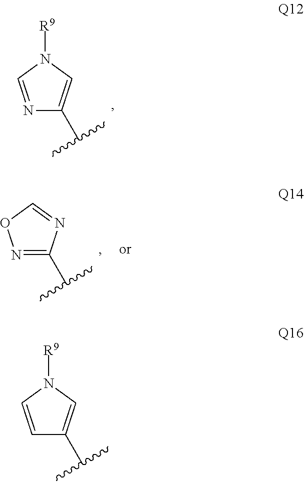

wherein any of said Q-groups is unsubstituted or substituted with 1, 2 or 3 substituents selected from C₁-C₄-alkyl, halogen, C₁-C₄-haloalkyl, or C₃-C₆cycloalkyl, and wherein R⁹ is selected from hydrogen or C₁-C₄ alkyl, wherein [symbol] denotes the position, which is attached to the tetrahydroisoquinoline-moiety.

In a more preferred embodiment of the invention, Q is selected from Q1, Q2, Q3, Q4, Q6, Q11, Q12, or Q16,

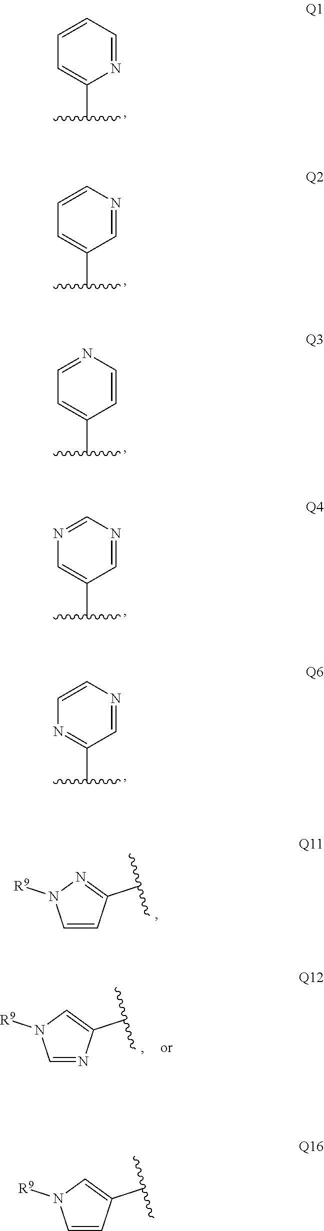

wherein any of said Q groups is unsubstituted or substituted with 1, 2 or 3 substituents selected from C₁-C₄-alkyl, halogen, C₁-C₄-haloalkyl, or C₃-C₅cycloalkyl, and wherein R⁹ is selected from hydrogen or methyl, and wherein [symbol] denotes the position, which is attached to the tetrahydroisoquinoline-moiety.

In an even more preferred embodiment of the invention, Q is selected from Q1, Q2, Q3, Q4, Q6, Q11, Q12, or Q16, wherein any of said Q groups is unsubstituted or substituted with 1, 2 or 3 substituents selected from C₁-C₄-alkyl, halogen, C₁-C₄-haloalkyl, or C₃-C₅cycloalkyl, and wherein R⁹ is methyl, and wherein [symbol] denotes the position, which is attached to the tetrahydroisoquinoline-moiety.

In a still more preferred embodiment of the invention, Q is selected from Q1, Q2, Q3, Q4, Q6, Q11, Q12, or Q16, wherein any of said Q groups is unsubstituted or substituted with 1 or 2 substituents selected from fluorine, chlorine, bromine, cyano, or methyl, and wherein R⁹ is methyl.

In a still even more preferred embodiment of the invention, Q is selected from Q1, Q2, Q3, Q4, Q6, Q11, or Q12, wherein any of said Q groups is unsubstituted or substituted with 1 or 2 substituents selected from fluorine, chlorine, bromine, cyano, or methyl, and wherein R⁹ is methyl.

In a still even more preferred embodiment of the invention, Q is selected from Q1, Q2, or Q3, wherein any of said Q groups is unsubstituted or substituted with 1 or 2 substituents selected from fluorine, chlorine, bromine, cyano, or methyl, and wherein R⁹ is methyl.

In a further still even more preferred embodiment of the invention, Q is selected from Q1, Q2, or Q3, wherein any of said Q groups is unsubstituted or substituted with 1 substituent selected from methyl.

Preferably O is selected from

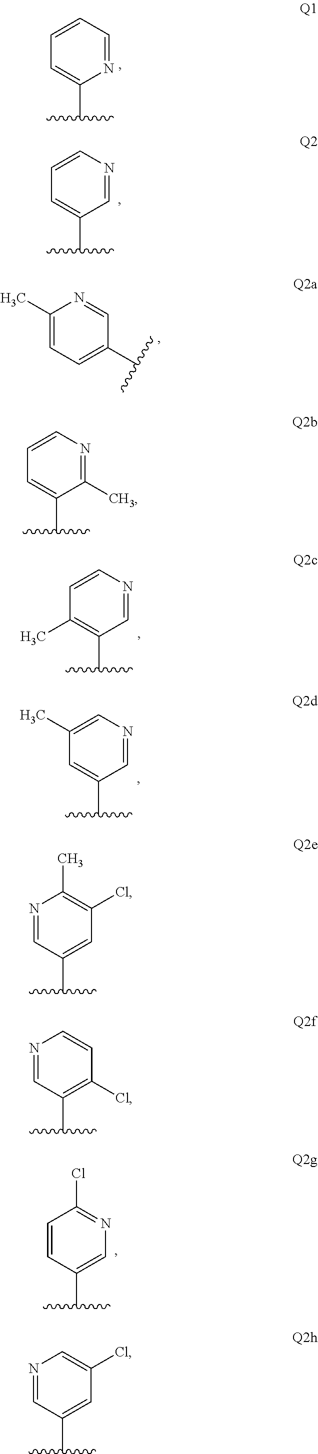

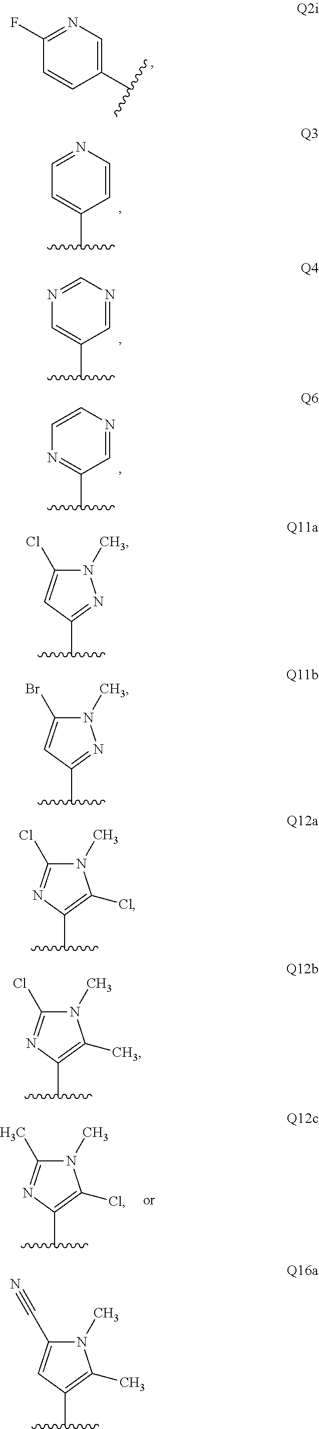

wherein [symbol] denotes the position, which is attached to the tetrahydroisoquinoline-moiety.

More preferably O is selected from

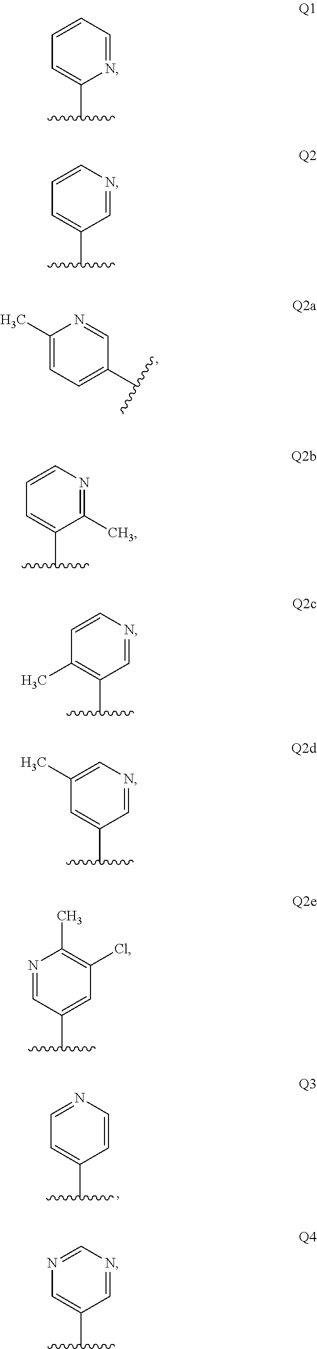

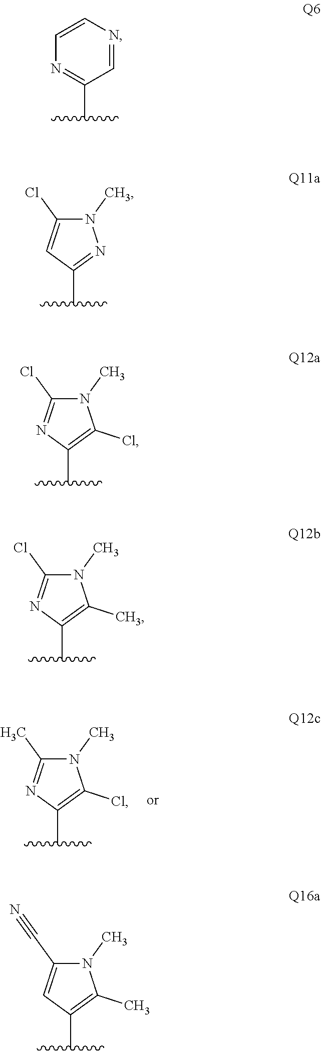

wherein [symbol] denotes the position, which is attached to the tetrahydroisoquinoline-moiety.

Even more preferably O is selected from

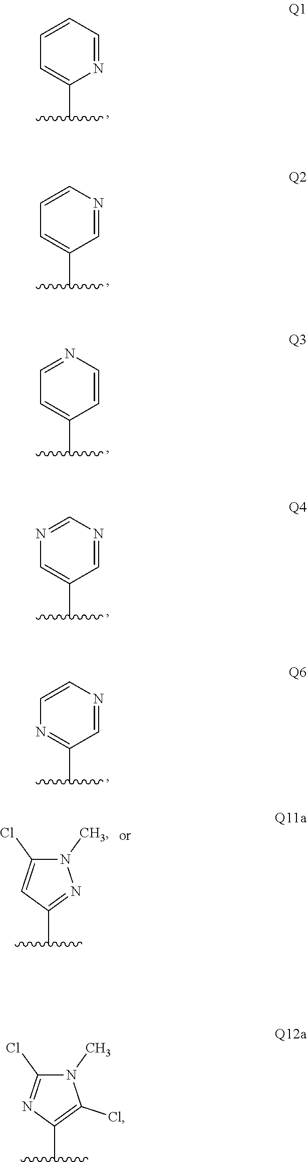

wherein [symbol] denotes the position, which is attached to the tetrahydroisoquinoline-moiety.

Most preferably, Q is selected from Q1, Q2a, Q3, or Q4

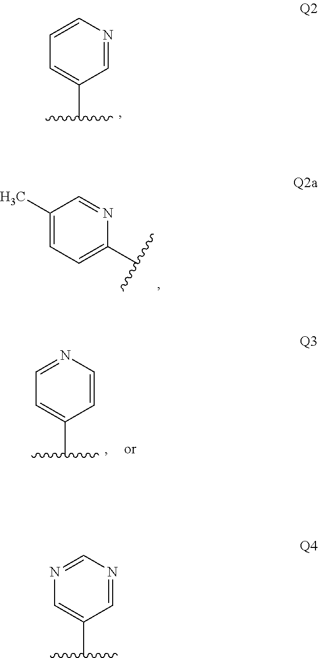

wherein [symbol] denotes the position, which is attached to the tetrahydroisoquinoline-moiety.

The present invention, accordingly, makes available a compound of formula (I) having R¹, R², R³, R⁴, R⁵, R⁶, Q, B¹, B², A¹, A², A³, A⁴ and Z¹ as defined above in all combinations/each permutation.

In one embodiment of the invention, the compound of formula (I) may be a compound of formula (I-A)

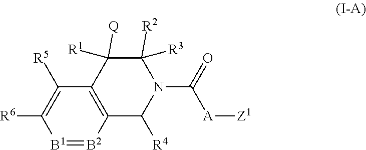

wherein
R¹, R², R³, R⁴, R⁵, B¹ and B², Q, and Z¹ are as defined for the compounds of formula (I) according to the present invention, and A is selected from A1 to A38:

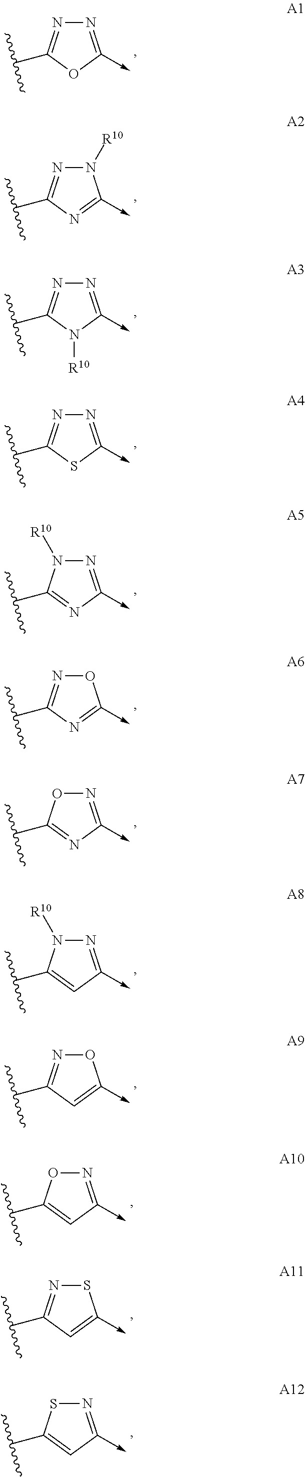

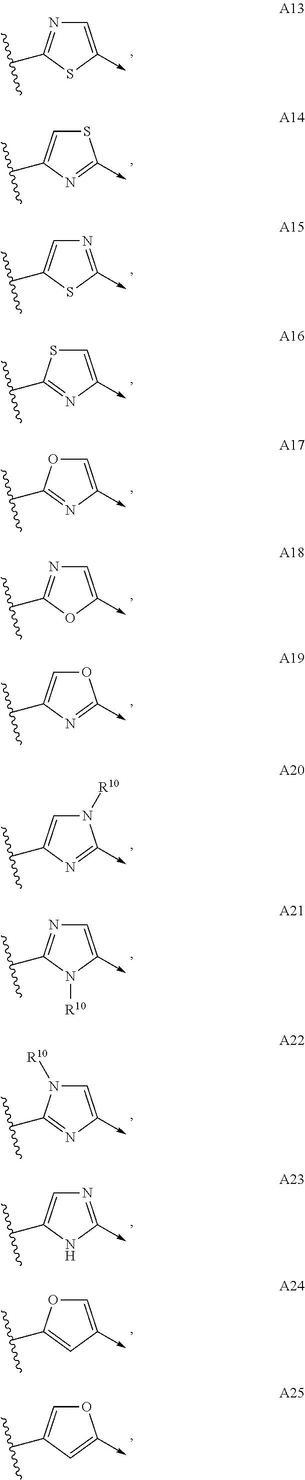

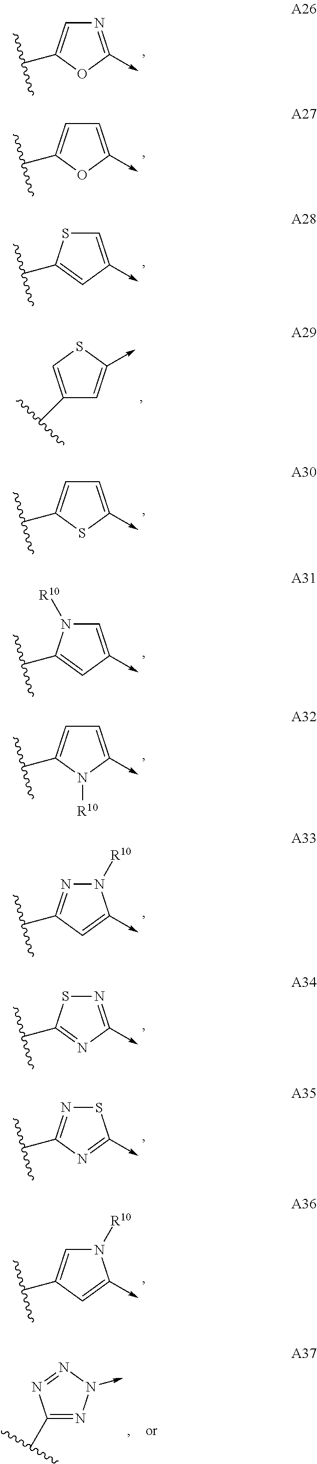

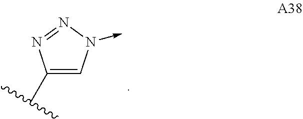

wherein [symbol] denotes the position, which is attached to the C(═O) group and the arrow denotes the position, which is attached to the Z¹ group, and wherein R¹, R², R³, R⁴, R⁵, R⁶, Q, B¹, B² and Z¹ are as defined for the compounds of formula (I) according to the present invention, and wherein R¹⁰ is selected from hydrogen or C₁-C₄ alkyl.

Preferably the compound of formula (I) may be a compound of formula (I-A), wherein R¹, R², R³, R⁴, R⁵, B¹ and B², Q, and Z¹ are as defined for the compounds of formula (I) according to the present invention, and A is selected from A1 to A38, and wherein R¹⁰ is selected from methyl.

In an embodiment of the invention, in the compound of formula (I-A), wherein R¹, R², R³, R⁴, R⁵, R⁶, Q, B¹, B² and Z¹ are as defined for the compounds of formula (I) according to the present invention, and A is selected from

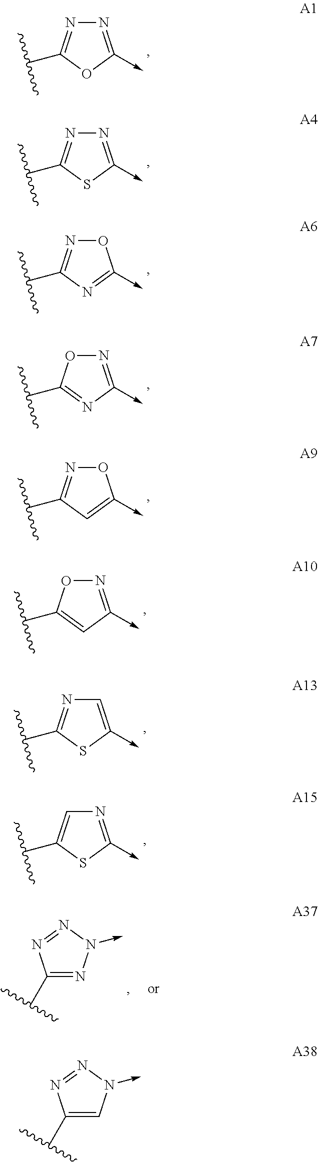

wherein [symbol] denotes the position, which is attached to the C(═O) group and the arrow denotes the position, which is attached to the Z¹ group.

In another embodiment of the invention, in the compound of formula (I-A), wherein R¹, R², R³, R⁴, R⁵, R⁶, Q, B¹, B² and Z¹ are as defined for the compounds of formula (I) according to the present invention, and A is selected from

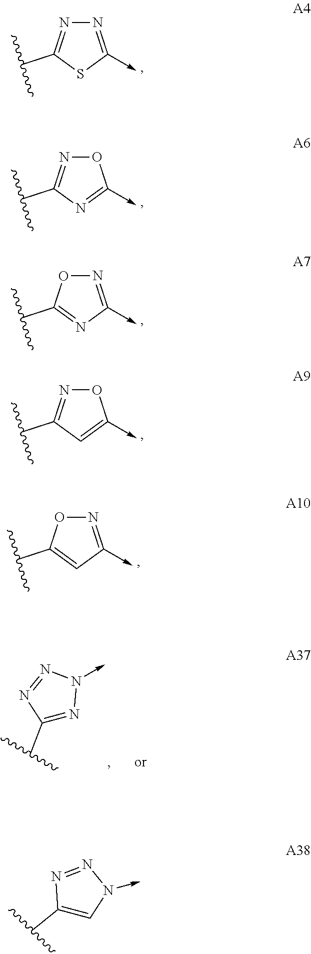

wherein [symbol] denotes the position, which is attached to the C(═O) group and the arrow denotes the position, which is attached to the Z¹ group.

In another preferred embodiment of the invention, in the compound of formula (I-A), wherein R¹, R², R³, R⁴, R⁵, R⁶, Q, B¹, B² and Z¹ are as defined for the compounds of formula (I) according to the present invention, and A is selected from

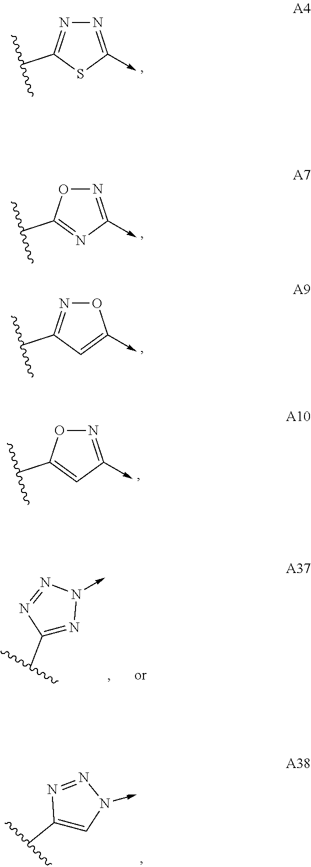

wherein [symbol] denotes the position, which is attached to the C(═O) group and the arrow denotes the position, which is attached to the Z¹ group.

The present invention, accordingly, makes available a compound of formula (I) having R¹, R², R³, R⁴, R⁵, R⁶, Q, B¹, B², A and Z¹ as defined above in all combinations/each permutation.

Embodiments according to the invention are provided as set out below.

In one embodiment of the invention, the compound of formula (I) may be a compound of formula (I-A), wherein
R¹ is hydrogen, or C₁-C₄ alkyl;
R², R³ are hydrogen;
R⁴ is hydrogen, C₁-C₄ alkyl, or C₃-C₆-cycloalkyl;
R⁵, R⁶ are independently selected from hydrogen, halogen, cyano, C₁-C₄ alkyl, or C₁-C₄ alkoxy;
B1 is N, or CR⁷;
B2 is N, or CR⁸;
R⁷ and R⁸ are independently selected from hydrogen or halogen; and
Q and Z¹ are as defined for compounds of formula (I) and A is as defined for compounds of formula (I-A).

In another embodiment of the invention, the compound of formula (I) may be a compound of formula (I-A), wherein
R¹ is hydrogen, or C₁-C₄ alkyl;
R², R³ are hydrogen;
R⁴ is hydrogen, C₁-C₄ alkyl, or C₃-C₆-cycloalkyl;
R⁵, R⁶ are independently selected from hydrogen, halogen, cyano, C₁-C₄ alkyl, or C₁-C₄ alkoxy;
B¹ is N, or CR⁷;
B² is N, or CR⁸;
R⁷ and R⁸ are independently selected from hydrogen or halogen;
Q is selected from Q1, Q2, Q3, Q4, Q6, Q11, Q12, or Q16, wherein any of said Q groups is unsubstituted or substituted with 1 or 2 substituents selected from fluorine, chlorine, bromine, cyano or methyl, and wherein R⁹ is methyl;
A is selected from A4, A6, A7, A9, A10, A37, or A39; and
Z¹ is as defined for compounds of formula (I).

In another preferred embodiment of the invention, the compound of formula (I) may be a compound of formula (I-A), wherein
R¹ is hydrogen, or C₁-C₄ alkyl;
R², R³ are hydrogen;
R⁴ is hydrogen, C₁-C₄ alkyl, or C₃-C₆-cycloalkyl;
R⁵, R⁶ are independently selected from hydrogen, halogen, cyano, C₁-C₄ alkyl, or C₁-C₄ alkoxy;
B¹ is N, or CR⁷ and B² is N, or CR⁸; wherein R⁷ and R⁸ are independently selected from hydrogen, halogen, C₁-C₃ alkyl, or C₁-C₄ alkoxy;
Q is selected from Q1, Q2, Q3, Q4, Q6, Q11, Q12, or Q16, wherein any of said Q-groups is unsubstituted or substituted with 1 or 2 substituents selected from fluorine, chlorine, bromine, cyano or methyl, and wherein R⁹ is methyl;
A is selected from A4, A6, A7, A9, A10, A37, or A39; and
Z¹ is selected from C₁-C₄ alkyl, phenyl, a 5- to 6-membered heteroaryl, or C₃-C₆-cycloalkyl, wherein said 5- or 6-membered heteroaryl contains 1 heteroatom selected from N, and wherein any of said phenyl and 5- to 6-membered heteroaryl are unsubstituted or substituted with 1 or 2 substituents independently selected from fluoro, chloro, methyl, ethyl, ethynyl, methoxy, or methylsulfonyl, and wherein said C₃-C₆-cycloalkyl is unsubstituted or substituted with 1 or 2 substituents selected from methyl.

In another preferred embodiment of the invention, the compound of formula (I) may be a compound of formula (I-A), wherein
R¹ is hydrogen, or C₁-C₄ alkyl;
R², R³ are hydrogen;
R⁴ is hydrogen, C₁-C₄ alkyl, or C₃-C₆-cycloalkyl;
R⁵, R⁶ are independently selected from hydrogen, halogen, cyano, C₁-C₄ alkyl, or C₁-C₄ alkoxy; B¹ is CR⁷ and B² is CR⁸; wherein R⁷ and R⁸ are independently selected from hydrogen or halogen; Q is selected from Q1, Q2, or Q3, wherein any of said Q groups is unsubstituted or substituted with 1 or 2 substituents selected from fluorine, chlorine, bromine, cyano or methyl, and wherein R⁹ is methyl;
A is selected from A4, A6, A7, A9, A10, A37, or A39; and
Z¹ is selected from C₁-C₄ alkyl, phenyl, a 5- to 6-membered heteroaryl, or C₃-C₆-cycloalkyl, wherein said 5- or 6-membered heteroaryl contains 1 heteroatom selected from N, and wherein any of said phenyl and 5- to 6-membered heteroaryl are unsubstituted or substituted with 1 or 2 substituents independently selected from fluoro, chloro, methyl, ethyl, ethynyl, methoxy, or methylsulfonyl, and wherein said C₃-C₆-cycloalkyl is unsubstituted or substituted with 1 or 2 substituents selected from methyl.

In a further preferred embodiment of the invention, the compound of formula (I) may be a compound of formula (I-A), wherein
R¹ is hydrogen, or C₁-C₄ alkyl;
R², R³ are hydrogen;
R⁴ is hydrogen, C₁-C₄ alkyl, or C₃-C₆-cycloalkyl;
R⁵, R⁶ are independently selected from hydrogen, halogen, cyano, C₁-C₄ alkyl, or C₁-C₄ alkoxy;
B¹ is CR⁷ and B² is CR^{B}; wherein R⁷ and R⁸ are independently selected from hydrogen or halogen;
Q is selected from Q2, Q3, or Q4, wherein any of said Q groups is unsubstituted or substituted with 1 substituent selected from methyl;
A is selected from A4, A7, A9, A10, A37, or A39; and
Z1 is selected from C₁-C₄ alkyl, phenyl, a 5- to 6-membered heteroaryl, or C₃-C₆-cycloalkyl, wherein said 5- or 6-membered heteroaryl contains 1 heteroatom selected from N, and wherein any of said phenyl and 5- to 6-membered heteroaryl are unsubstituted or substituted with 1 or 2 substituents independently selected from fluoro, chloro, methyl, ethyl, ethynyl, methoxy, or methylsulfonyl, and wherein said C₃-C₆-cycloalkyl is unsubstituted or substituted with 1 or 2 substituents selected from methyl.

In still a further preferred embodiment of the invention, the compound of formula (I) may be a compound of formula (I-A), wherein
R¹ is hydrogen, or C₁-C₄ alkyl;
R², R³ are hydrogen;
R⁴ is hydrogen, C₁-C₄ alkyl, or C₃-C₆-cycloalkyl;
R⁵, R⁶ are independently selected from hydrogen, halogen, cyano, C₁-C₄ alkyl, or C₁-C₄ alkoxy;
B¹ is CR⁷ and B² is CR⁸; wherein R⁷ and R⁸ are independently selected from hydrogen or halogen;
Q is selected from Q2, Q3, or Q4, wherein any of said Q groups is unsubstituted or substituted with 1 substituent selected from methyl;
A is selected from A4, A7, A9, A10, A37, or A39; and
Z¹ is 2,4-difluorophenyl, 3,5-difluoro-2-pyridyl, 5-fluoro-2-pyridyl, 3-fluoro-2-pyridyl, 3,5-difluoro-2-furyl, 3-fluoro-2-furyl, 5-fluoro-2-furyl, 3,5-difluoro-2-thienyl, 3-fluoro-2-thienyl, 5-fluoro-2-thienyl, 2-fluorophenyl, 4-fluorophenyl, or phenyl.

In still another preferred embodiment of the invention, the compound of formula (I) may be a compound of formula (I-A), wherein
R¹ is hydrogen, or C₁-C₄ alkyl;
R², R³ are hydrogen;
R⁴ is hydrogen, C₁-C₄ alkyl, or C₃-C₆-cycloalkyl;
R⁵, R⁶ are independently selected from hydrogen, halogen, cyano, C₁-C₄ alkyl, or C₁-C₄ alkoxy;
B¹ is CH and B² is CH;
Q is selected from Q2, Q3, or Q4, wherein any of said Q groups is unsubstituted or substituted with 1 substituent selected from methyl;
A is selected from A4, A7. A9, A10, A37, or A39; and
Z¹ is 2,4-difluorophenyl, 3,5-difluoro-2-pyridyl, 5-fluoro-2-pyridyl, 3-fluoro-2-pyridyl, 3,5-difluoro-2-furyl, 3-fluoro-2-furyl, 5-fluoro-2-furyl, 3,5-difluoro-2-thienyl, 3-fluoro-2-thienyl, 5-fluoro-2-thienyl, 2-fluorophenyl, 4-fluorophenyl, or phenyl.

In one embodiment of the invention, the compound of formula (I-A) may be a compound of formula (I-A1) wherein B¹ is CH, B² is CH, and A is defined as for compound (I-A),

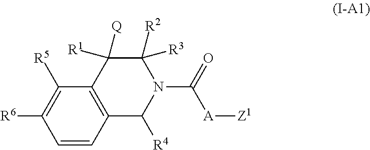

wherein R¹, R², R³, R⁴, R⁵, R⁶, Q, A and Z¹ are as defined for compounds of formula (I).

Preferably in a compound of formula (I-A1)
R¹ is hydrogen, or methyl;
R², R³ are hydrogen;
R⁴ is hydrogen, C₁-C₄ alkyl, or C₃-C₆-cycloalkyl;
R⁵, R⁶ are independently selected from hydrogen, halogen, cyano, C₁-C₄ alkyl, or C₁-C₄ alkoxy;
Q is selected from Q1, Q2, Q3, Q4, Q6, Q11, Q12, or Q16, wherein any of said Q groups is unsubstituted or substituted with 1 or 2 substituents selected from fluorine, chlorine, bromine, cyano or methyl, and wherein R⁹ is methyl;
A is selected from A4, A6, A7, A9, A10, A37 or A39; and
Z¹ is as defined for compounds of formula (I).

More preferably in a compound of formula (I-A1)
R¹ is hydrogen, or methyl;
R², R³ are hydrogen;
R⁴ is hydrogen, C₁-C₄ alkyl, or C₃-C₆-cycloalkyl;
R⁵, R⁶ are independently selected from hydrogen, halogen, cyano, C₁-C₄ alkyl, or C₁-C₄ alkoxy;
Q is selected from Q2, Q3, or Q4, wherein any of said Q groups is unsubstituted or substituted with 1 substituent selected from methyl;
A is selected from A4, A7, A9, A10, A37 or A39; and
Z¹ is selected from C₁-C₄ alkyl, phenyl, a 5- to 6-membered heteroaryl, or C₃-C₆-cycloalkyl, wherein said 5- or 6-membered heteroaryl contains 1 heteroatom selected from N, and wherein any of said phenyl and 5- to 6-membered heteroaryl are unsubstituted or substituted with 1 or 2 substituents independently selected from fluoro, chloro, methyl, ethyl, ethynyl, methoxy, or methylsulfonyl, and wherein said C₃-C₆-cycloalkyl is unsubstituted or substituted with 1 or 2 substituents selected from methyl.

In another embodiment of the invention, the compound of formula (I-A) may be a compound of formula (I-A2) wherein B¹ is CH, B² is CH, and A is A4,

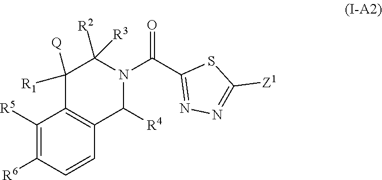

wherein R¹, R², R³, R⁴, R⁵, R⁶, Q, A and Z¹ are as defined for compounds of formula (I).

Preferably in a compound of formula (I-A2)
R¹ is hydrogen, or methyl;
R², R³ are hydrogen;
R⁴ is hydrogen, C₁-C₄ alkyl, or C₃-C₆-cycloalkyl;
R⁵, R⁶ are independently selected from hydrogen, halogen, cyano, C₁-C₄ alkyl, or C₁-C₄ alkoxy;
Q is selected from Q1, Q2, Q3, Q4, Q6, Q11, Q12, or Q16, wherein any of said Q groups is unsubstituted or substituted with 1 or 2 substituents selected from fluorine, chlorine, bromine, cyano or methyl, and wherein R⁹ is methyl; and
Z¹ is as defined for compounds of formula (I).

More preferably in a compound of formula (I-A2)
R¹ is hydrogen, or methyl;
R², R³ are hydrogen;
R⁴ is hydrogen, C₁-C₄ alkyl, or C₃-C₆-cycloalkyl;
R⁵, R⁶ are independently selected from hydrogen, halogen, cyano, C₁-C₄ alkyl, or C₁-C₄ alkoxy;
Q is selected from Q2, Q3, or Q4, wherein any of said Q groups is unsubstituted or substituted with 1 substituent selected from methyl; and
Z¹ is selected from C₁-C₄ alkyl, phenyl, a 5- to 6-membered heteroaryl, or C₃-C₆-cycloalkyl, wherein said 5- or 6-membered heteroaryl contains 1 heteroatom selected from N, and wherein any of said phenyl and 5- to 6-membered heteroaryl are unsubstituted or substituted with 1 or 2 substituents independently selected from fluoro, chloro, methyl, ethyl, ethynyl, methoxy, or methylsulfonyl, and wherein said C₃-C₆-cycloalkyl is unsubstituted or substituted with 1 or 2 substituents selected from methyl.

In another embodiment of the invention, the compound of formula (I-A) may be a compound of formula (I-A3) wherein B¹ is CH, B² is CH, and A is A7,

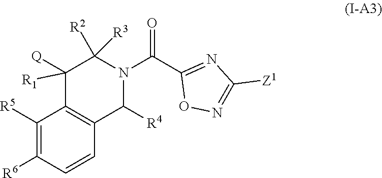

wherein R¹, R², R³, R⁴, R⁵, R⁶, Q, A and Z¹ are as defined for compounds of formula (I).

Preferably in a compound of formula (I-A3)
R¹ is hydrogen, or methyl;
R², R³ are hydrogen;
R⁴ is hydrogen, C₁-C₄ alkyl, or C₃-C₆-cycloalkyl;
R⁵, R⁶ are independently selected from hydrogen, halogen, cyano, C₁-C₄ alkyl, or C₁-C₄ alkoxy;
Q is selected from Q1, Q2, Q3, Q4, Q6, Q11, Q12, or Q16, wherein any of said Q groups is unsubstituted or substituted with 1 or 2 substituents selected from fluorine, chlorine, bromine, cyano or methyl, and wherein R⁹ is methyl; and
Z¹ is as defined for compounds of formula (I).

More preferably in a compound of formula (I-A3)
R¹ is hydrogen, or methyl;
R², R³ are hydrogen;
R⁴ is hydrogen, C₁-C₄ alkyl, or C₃-C₆-cycloalkyl;
R⁵, R⁶ are independently selected from hydrogen, halogen, cyano, C₁-C₄ alkyl, or C₁-C₄ alkoxy;
Q is selected from Q2, Q3, or Q4, wherein any of said Q groups is unsubstituted or substituted with 1 substituent selected from methyl; and
Z¹ is selected from C₁-C₄ alkyl, phenyl, a 5- to 6-membered heteroaryl, or C₃-C₆-cycloalkyl, wherein said 5- or 6-membered heteroaryl contains 1 heteroatom selected from N, and wherein any of said phenyl and 5- to 6-membered heteroaryl are unsubstituted or substituted with 1 or 2 substituents independently selected from fluoro, chloro, methyl, ethyl, ethynyl, methoxy, or methylsulfonyl, and wherein said C₃-C₆-cycloalkyl is unsubstituted or substituted with 1 or 2 substituents selected from methyl.

In another embodiment of the invention, the compound of formula (I-A) may be a compound of formula (I-A4) wherein B¹ is CH, B² is CH, and A is A9,

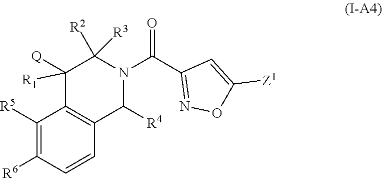

wherein R¹, R², R³, R⁴, R⁵, R⁶, Q, A and Z¹ are as defined for compounds of formula (I).

Preferably in a compound of formula (I-A4)
R¹ is hydrogen, or methyl;
R², R³ are hydrogen;
R⁴ is hydrogen, C₁-C₄ alkyl, or C₃-C₆-cycloalkyl;
R⁵, R⁶ are independently selected from hydrogen, halogen, cyano, C₁-C₄ alkyl, or C₁-C₄ alkoxy;
Q is selected from Q1, Q2, Q3, Q4, Q6, Q11, Q12, or Q16, wherein any of said Q groups is unsubstituted or substituted with 1 or 2 substituents selected from fluorine, chlorine, bromine, cyano or methyl, and wherein R⁹ is methyl; and
Z¹ is as defined for compounds of formula (I).

More preferably in a compound of formula (I-A4)
R¹ is hydrogen, or methyl;
R², R³ are hydrogen;
R⁴ is hydrogen, C₁-C₄ alkyl, or C₃-C₆-cycloalkyl;
R⁵, R⁶ are independently selected from hydrogen, halogen, cyano, C₁-C₄ alkyl, or C₁-C₄ alkoxy;
Q is selected from Q2, Q3, or Q4, wherein any of said Q groups is unsubstituted or substituted with 1 substituent selected from methyl; and
Z¹ is selected from C₁-C₄ alkyl, phenyl, a 5- to 6-membered heteroaryl, or C₃-C₆-cycloalkyl, wherein said 5- or 6-membered heteroaryl contains 1 heteroatom selected from N, and wherein any of said phenyl and 5- to 6-membered heteroaryl are unsubstituted or substituted with 1 or 2 substituents independently selected from fluoro, chloro, methyl, ethyl, ethynyl, methoxy, or methylsulfonyl, and wherein said C₃-C₆-cycloalkyl is unsubstituted or substituted with 1 or 2 substituents selected from methyl.

In another embodiment of the invention, the compound of formula (I-A) may be a compound of formula (I-A5) wherein B¹ is CH, B² is CH, and A is A10,

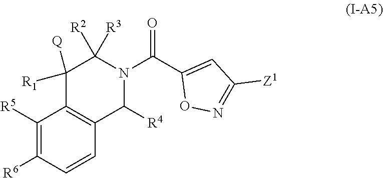

wherein R¹, R², R³, R⁴, R⁵, R⁶, Q, A and Z¹ are as defined for compounds of formula (I).

Preferably in a compound of formula (I-A5)
R¹ is hydrogen, or methyl;
R², R³ are hydrogen;
R⁴ is hydrogen, C₁-C₄ alkyl, or C₃-C₆-cycloalkyl;
R⁵, R⁶ are independently selected from hydrogen, halogen, cyano, C₁-C₄ alkyl, or C₁-C₄ alkoxy;
Q is selected from Q1, Q2, Q3, Q4, Q6, Q11, Q12, or Q16, wherein any of said Q groups is unsubstituted or substituted with 1 or 2 substituents selected from fluorine, chlorine, bromine, cyano or methyl, and wherein R⁹ is methyl; and
Z¹ is as defined for compounds of formula (I).

More preferably in a compound of formula (I-A5)
R¹ is hydrogen, or methyl;
R², R³ are hydrogen;
R⁴ is hydrogen, C₁-C₄ alkyl, or C₃-C₆-cycloalkyl;
R⁵, R⁶ are independently selected from hydrogen, halogen, cyano, C₁-C₄ alkyl, or C₁-C₄ alkoxy;
Q is selected from Q2, Q3, or Q4, wherein any of said Q groups is unsubstituted or substituted with 1 substituent selected from methyl; and
Z¹ is selected from C₁-C₄ alkyl, phenyl, a 5- to 6-membered heteroaryl, or C₃-C₆-cycloalkyl, wherein said 5- or 6-membered heteroaryl contains 1 heteroatom selected from N, and wherein any of said phenyl and 5- to 6-membered heteroaryl are unsubstituted or substituted with 1 or 2 substituents independently selected from fluoro, chloro, methyl, ethyl, ethynyl, methoxy, or methylsulfonyl, and wherein said C₃-C₆-cycloalkyl is unsubstituted or substituted with 1 or 2 substituents selected from methyl.

In another embodiment of the invention, the compound of formula (I-A) may be a compound of formula (I-A6) wherein B¹ is CH, B² is CH, and A is A37,

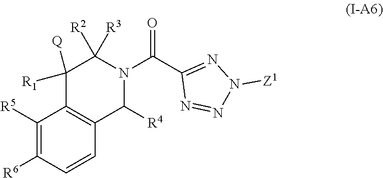

wherein R¹, R², R³, R⁴, R⁵, R⁶, Q, A and Z¹ are as defined for compounds of formula (I).

Preferably in a compound of formula (I-A6)
R¹ is hydrogen, or methyl;
R², R³ are hydrogen;
R⁴ is hydrogen, C₁-C₄ alkyl, or C₃-C₆-cycloalkyl;
R⁵, R⁶ are independently selected from hydrogen, halogen, cyano, C₁-C₄ alkyl, or C₁-C₄ alkoxy;
Q is selected from Q1, Q2, Q3, Q4, Q6, Q11, Q12, or Q16, wherein any of said Q groups is unsubstituted or substituted with 1 or 2 substituents selected from fluorine, chlorine, bromine, cyano or methyl, and wherein R⁹ is methyl; and
Z¹ is as defined for compounds of formula (I).

More preferably in a compound of formula (I-A6)
R¹ is hydrogen, or methyl;
R², R³ are hydrogen;
R⁴ is hydrogen, C₁-C₄ alkyl, or C₃-C₆-cycloalkyl;
R⁵, R⁶ are independently selected from hydrogen, halogen, cyano, C₁-C₄ alkyl, or C₁-C₄ alkoxy;
Q is selected from Q2, Q3, or Q4, wherein any of said Q groups is unsubstituted or substituted with 1 substituent selected from methyl; and
Z¹ is selected from C₁-C₄ alkyl, phenyl, a 5- to 6-membered heteroaryl, or C₃-C₆-cycloalkyl, wherein said 5- or 6-membered heteroaryl contains 1 heteroatom selected from N, and wherein any of said phenyl and 5- to 6-membered heteroaryl are unsubstituted or substituted with 1 or 2 substituents independently selected from fluoro, chloro, methyl, ethyl, ethynyl, methoxy, or methylsulfonyl, and wherein said C₃-C₆-cycloalkyl is unsubstituted or substituted with 1 or 2 substituents selected from methyl.

In another embodiment of the invention, the compound of formula (I-A) may be a compound of formula (I-A7) wherein R¹ B¹ is CH, B² is CH, and A is A38,

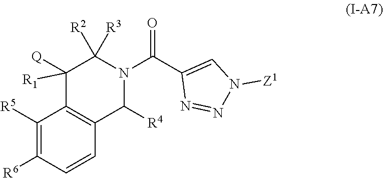

wherein R¹, R², R³, R⁴, R⁵, R⁶, Q, A and Z¹ are as defined for compounds of formula (I).

Preferably in a compound of formula (I-A7)
R¹ is hydrogen, or methyl;
R², R³ are hydrogen;
R⁴ is hydrogen, C₁-C₄ alkyl, or C₃-C₆-cycloalkyl;
R⁵, R⁶ are independently selected from hydrogen, halogen, cyano, C₁-C₄ alkyl, or C₁-C₄ alkoxy;
Q is selected from Q1, Q2, Q3, Q4, Q6, Q11, Q12, or Q16, wherein any of said Q groups is unsubstituted or substituted with 1 or 2 substituents selected from fluorine, chlorine, bromine, cyano or methyl, and wherein R⁹ is methyl; and
Z¹ is as defined for compounds of formula (I).

More preferably in a compound of formula (I-A7)
R¹ is hydrogen, or methyl;
R², R³ are hydrogen;
R⁴ is hydrogen, C₁-C₄ alkyl, or C₃-C₆-cycloalkyl;
R⁵, R⁶ are independently selected from hydrogen, halogen, cyano, C₁-C₄ alkyl, or C₁-C₄ alkoxy;
Q is selected from Q2, Q3, or Q4, wherein any of said Q groups is unsubstituted or substituted with 1 substituent selected from methyl; and
Z¹ is selected from C₁-C₄ alkyl, phenyl, a 5- to 6-membered heteroaryl, or C₃-C₆-cycloalkyl, wherein said 5- or 6-membered heteroaryl contains 1 heteroatom selected from N, and wherein any of said phenyl and 5- to 6-membered heteroaryl are unsubstituted or substituted with 1 or 2 substituents independently selected from fluoro, chloro, methyl, ethyl, ethynyl, methoxy, or methylsulfonyl, and wherein said C₃-C₆-cycloalkyl is unsubstituted or substituted with 1 or 2 substituents selected from methyl.

The presence of one or more possible asymmetric carbon atoms in any of the compounds selected from compounds of formula (I), (I-A), (I-A1), (I-A2), (I-A3), (I-A4), (I-A5), (I-A6), or (I-A7), or compounds selected from compounds listed in Tables C-1 to C-36, or compounds listed in Table P (below), according to the invention means that the compounds may occur in chiral isomeric forms, i.e., enantiomeric or diastereomeric forms.

In one embodiment, the compound of formula (I) according to the invention is selected from compounds listed in any one of Tables C-1 to C-36.

In another embodiment, the compound of formula (I) according to the invention is selected from compounds as listed in Table P (below).

The compounds of formula (I) according to the present invention can be made as shown in the following Schemes below, in which, unless otherwise stated, the definition of each variable is as defined above for a compound of formula (I).

In any of the Schemes below, the presence of one or more possible asymmetric carbon atoms in a compound of formula (I) according to the invention means that the compounds may occur in chiral isomeric forms, i.e., enantiomeric or diastereomeric forms.

Compounds of formula (I) may be prepared by a person skilled in the art following known methods. More specifically, ccompounds of formula (I) may be prepared from compounds of formula (III) or a salt thereof, wherein R¹, R², R³, R⁴, R⁵, R⁶, Q, B¹ and B² are as defined for the compounds of formula (I) by reaction with a compound of formula (II), wherein A¹, A², A³, A⁴ and Z¹ are as defined for the compounds of formula (I). This reaction is shown in Scheme 1.

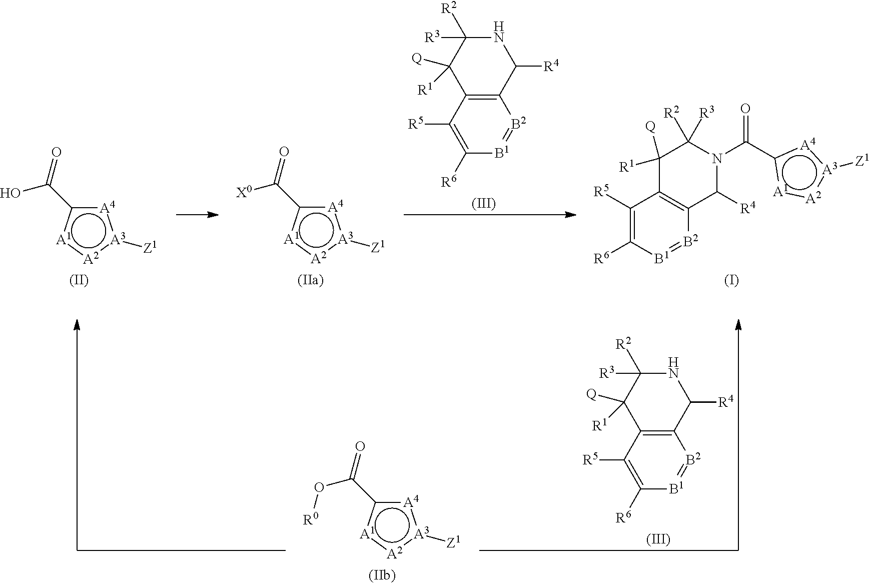

In Scheme 1, compounds of formula (II), wherein A¹, A², A³, A⁴ and Z¹ are as defined for the compounds of formula (I), are activated to compounds of formula (IIa) by methods known to a person skilled in the art and described, for example, in Tetrahedron 2005, 61 (46), 10827-10852. For example, compounds of formula (IIa), where X⁰ is halogen, are formed by treatment of compounds of formula (II) with, for example, oxalyl chloride or thionyl chloride in the presence of catalytic quantities of N,N-dimethylformamide (DMF) in inert solvents such as dichloromethane (DCM) or tetrahydrofuran (THF) at temperatures between 20° C. to 100° C., preferably 25° C. Treatment of compounds of formula (IIa) with compounds of formula (III), wherein R¹, R², R³, R⁴, R⁵, R⁶, Q, B¹ and B² are as defined for the compounds of formula (I), optionally in the presence of a base, e.g. triethylamine or pyridine, leads to compounds of formula (I). Alternatively, compounds of formula (I) may be prepared by treatment of compounds of formula (II) with dicyclohexyl carbodiimide (DCC), 1-ethyl-3-(3-dimethylaminopropyl)carbodiimide (EDC) or 1-[bis(dimethylamino)methylene]-1H-1,2,3-triazolo[4,5-b]pyridinium 3-oxide hexafluorophosphate (HATU) to give the activated compound of formula (IIa), wherein X° is G¹, G² or G3 as set forth in Scheme 2, in an inert solvent, e.g. pyridine, DMF, acetonitrile, DCM or THF, optionally in the presence of a base, e.g. triethylamine, at temperatures between 30° C. and 180° C. Finally, a compound of formula (II) can also be activated by reaction with a coupling reagent such as propanephosphonic acid anhydride (T3P) to provide compounds of formula (IIa), wherein X⁰ is G⁴ as set forth in Scheme 2, as described for example in Synthesis 2013, 45, 1569. Further reaction with an amine (or a salt thereof) of the compound of formula (III) leads to compounds of formula (I).

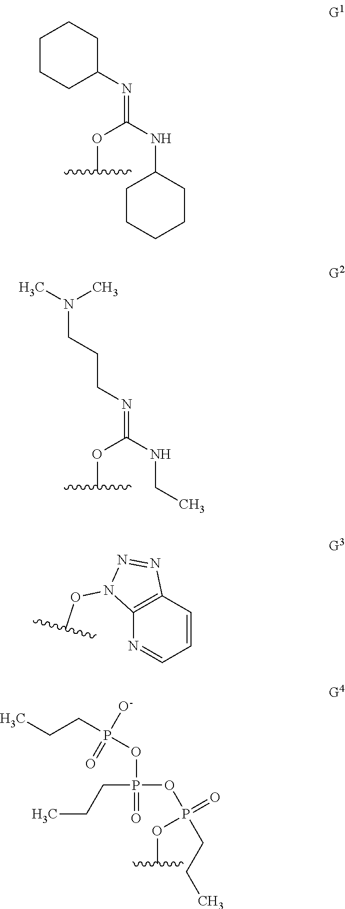

wherein [symbol] indicates the bond to the C(═O) group

Compounds of formula (II) can be prepared from compounds of formula (IIb), wherein A¹, A², A³, A⁴ and Z¹ are as described in formula (I), and R⁰ is C₁-C₄alkyl, by ester hydrolysis. A variety of conditions can be used, as for example aqueous sodium hydroxide or lithium hydroxide and an organic water miscible solvent like THE or dimethoxyethane, methanol or ethanol. Such ester hydrolyses are well known to those skilled in the art. Compounds of formula (IIb) can also be directly converted to compounds of formula (I) by reacting compounds of formula (IIb) with compounds of formula (III) in the presence of trimethyl aluminium, or trimethyl aluminium-DABCO complex in an inert solvent such as toluene or DCM. Such reactions have been reported in the literature (see Tetrahedron Lett. 1977, 4171-4174, Tetrahedron Lett. 2006, 5767-5769, and references cited therein). Compounds of formula (II) and (IIb) are commercially available or can be synthesized as described vide infra.

Compounds of formula (Illa), wherein R¹ and R² are hydrogen, R³ is hydrogen or methyl and R⁴, R⁵, R⁶, B¹ and B² are as defined for the compounds of formula (I), may be prepared from compounds of formula (IV), wherein R³ is hydrogen or methyl and R⁴, R⁵, R³, R⁶, B¹ and B² are as defined for the compounds of formula (I), by treatment with a reducing agent such as NaBH₃CN and an acid, for example hydrochloric acid, or acetic acid in a protic solvent such as methanol or ethanol and the like. Such reactions are well known in the literature and analogous reactions have been described for example in Deng, Zeping et al, CN103772278, and Synthesis 1979, 4, 281-283. Alternatively, compounds of formula (IIIa) may be prepared from compounds of formula (IV) by reduction with hydrogen in the presence of a suitable metal catalyst, such as Pd, Ir, Rh with a suitable ligand, e.g. diphosphine [1,2-bis(diphenylphosphino)ethane (dppe), 1,3-bis(diphenylphosphino)propane (dppp) or 1,4-bis(diphenylphosphino)butane (dppb)]. Similar reactions have been reported for example in React. Kinet. Cat. Lett. 2007, 92, 99-104. This reaction is shown in Scheme 3.

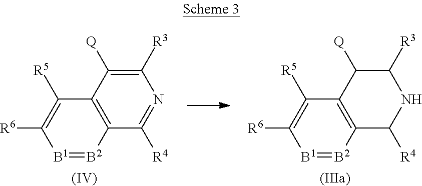

A further synthesis of compounds of formula (III), can be achieved by a directed metallation strategy as shown in Scheme 4, Thus, compounds of formula (IIIb) wherein R³ is hydrogen or methyl and R⁵, R⁶, Q, B¹ and B² are as defined forthe compounds of formula (I), are converted to compounds of formula (V), wherein R³ is hydrogen or methyl and R⁵, R⁶, Q, B¹ and B² are as defined for the compounds of formula (I), by treatment of compounds of formula (IIIb) with a compound of formula (VI), wherein X⁰ is a leaving group, such as halogen, and R⁰ is C₁-C₆alkyl, by methods known to a person skilled in the art and by those described in Scheme 1. Alternatively, compounds of formula (V) may be prepared by treatment with an anhydride of formula (R⁰CO)₂O, wherein R⁰ is C₁-C₆alkyl, in an inert solvent such as DCM, THF or 2-methyl-THF, optionally in the presence of a base, such as triethylamine or dimethylaminopyridine at temperatures between 0° C. and 60° C. Compounds of formula (V) are then metalated with a base, for example an alkyl metal base, such as tert-butyl lithium, and an additive such as N,N,N,N′-tetramethylethylendiamine (TMEDA) at low temperature, for example −78° C. to room temperature, in an inert polar solvent such as THF or 2-methyl-THF. Subsequent treatment of the anion of formula (V) formed under such conditions with an electrophile of formula R⁴—X⁰, wherein X⁰ is as previously defined and R⁴ is C₁-C₄ alkyl, C₁-C₄ alkylcarbonyl, C₁-C₄ alkoxycarbonyl, N-methoxy-N-methyl-carbonyl, C₁-C₄ alkylaminocarbonyl, di(C₁-C₄alkyl)aminocarbonyl, or C₃-C₆ cycloalkyl, wherein said C₃-C₅-cycloalkyl is unsubstituted or substituted by 1, 2 or 3 substituents independently selected from halogen, cyano, C₁-C₄ alkyl, C₁-C₄ haloalkyl, or C₁-C₄ alkoxy, yields compounds of formula (Va), wherein R⁰ is C₁-C₆ alkyl and R³ is halogen, and R⁴, R⁵, R⁶, Q, B¹ and B² are as defined for the compounds of formula (I) (Scheme 4).

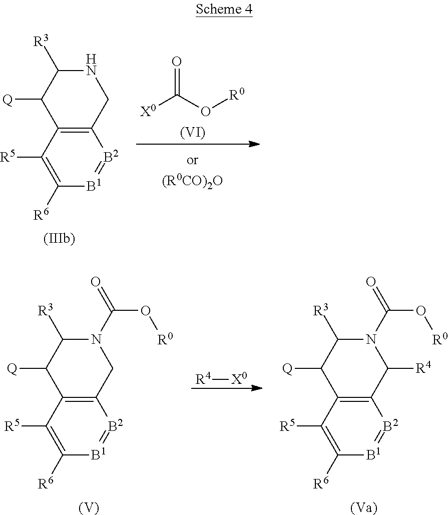

Compounds of formula (Va) may be converted to compounds of formula (Illa), wherein R³ is hydrogen or methyl and R⁴, R⁵, R⁶, Q, B¹ and B² are as defined for the compounds of formula (I), by methods known to a person skilled in the art. For example, compounds of formula (Va), wherein R⁰ is tert-butyl, may be treated with an organic or inorganic acid such as trifluoroacetic acid or HCl to give compounds of formula (Illa). This reaction is shown in Scheme 5.

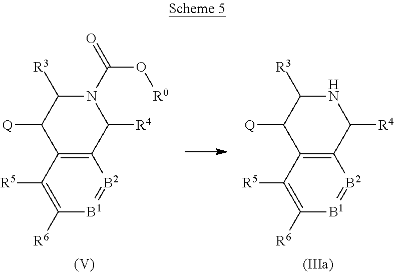

Compounds of formula (IV), wherein R³ is hydrogen or methyl and R⁴, R⁵, R⁶, Q, B¹ and B² are as defined for the compounds of formula (I), may be prepared by reacting compounds of formula (VIII), wherein Q is as defined for the compounds of formula (I) and X⁰ is halogen, preferably chlorine, bromine or iodine, with compounds of formula (VII), wherein R³ is hydrogen or methyl and R⁴, R⁵, R⁶, Q, B¹ and B² are as defined for the compounds of formula (I), by means of a C—C bond formation reaction typically under palladium-catalyzed (alternatively nickel-catalyzed) cross-coupling conditions. This reaction is shown in Scheme 6.

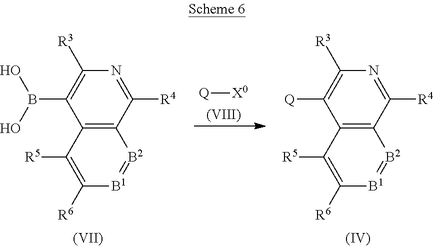

Suzuki-Miyaura cross-coupling reactions between compounds of formula (VIII) and compound of formula (VII) are well known to a person skilled in the art and are usually carried out in the presence of a palladium catalyst, such as tetrakis(triphenylphosphine)-palladium(0) or [1,1′-bis(diphenylphosphino) ferrocene]palladium(II) dichloride dichloromethane complex, and a base, such as sodium or potassium carbonate, in a solvent, such as N,N-dimethylformamide, dioxane or dioxane-water mixtures, at temperatures between room temperature and 160° C., optionally under microwave heating conditions, and preferably under inert atmosphere. Such reactions have been reviewed for example in J. Organomet. Chem. 1999, 576, 147-168. A person skilled in the art will also recognize that the reaction can be reversed, i.e. by reacting a compound of formula (X), wherein Q is as defined for the compounds of formula (I), with a compound of formula (IX), wherein R³ is hydrogen or methyl, R⁴, R⁵, R⁶, B¹ and B² are as defined for the compounds of formula (I) and X⁰ is halogen, preferably chlorine, bromine or iodine, to provide a compound of formula (IV), wherein R⁵ is hydrogen or methyl and R⁴, R⁵, R⁶, Q, B¹ and B² are as defined for the compounds of formula (I). This reaction is shown in Scheme 7.

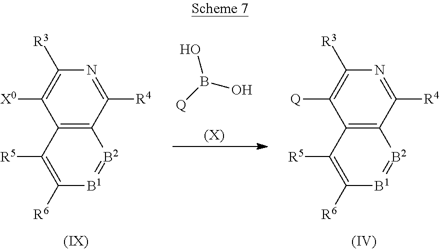

A further cross-coupling chemistry, namely C—H activation, can also be used to prepare compounds of formula (IV), wherein R³ is hydrogen or methyl and R⁴, R⁵, R⁶, Q, B¹ and B² are as defined for the compounds of formula (I), and wherein the Q group has at least one aromatic C—H group (Scheme 8).

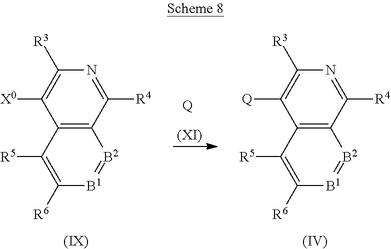

As shown in Scheme 8, compounds of formula (IX), wherein R³ is hydrogen or methyl, R⁴, R⁵, R⁶, B¹ and B² are as defined for the compounds of formula (I) and X⁰ is halogen, preferably chlorine, bromine or iodine, are reacted with compounds of formula (XI), wherein Q is as defined for the compounds of formula (I), and wherein the Q group has at least one aryl C—H, in the presence of a palladium catalyst, typically palladium acetate Pd(OAc)₂, a suitable ligand, for example 1,10-phenanthroline, in the presence of a base such as cesium carbonate or potassium carbonate, in inert solvents such as chlorobenzene, toluene or xylene at temperatures between room temperature and 180° C., optionally under microwave heating conditions, preferably under inert atmosphere. Similar reactions have been reported in the literature for example in Chem. Sci. 2013, 4, 2374-2379.

Compounds of formula (III) may also be prepared from compounds of formula (Vc) (Scheme 9).

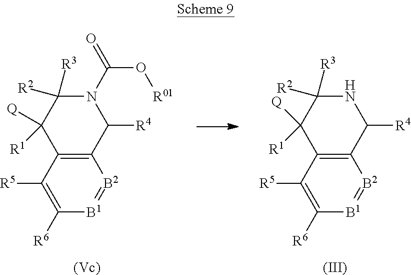

As shown in Scheme 9, compounds of formula (III) may be prepared by a person skilled in the art by a carbamate deprotection reaction of compounds of formula (Vc), wherein R¹, R², R³, R⁴, R⁵, R⁶, Q, B¹ and B² are as defined for the compounds of formula (I) and R⁰¹ may be a member of a common carbamate protecting group substituent, for example methyl, tert-butyl, allyl, 2,2,2-trichloroethyl or benzyl. For example, when R⁰¹ is methyl, a suitable solvent such as DCM and a suitable reagent such as iodotrimethylsilane may be employed to afford the product upon heating at temperatures between room temperature and 200° C., preferably between 20° C. and the boiling point of the reaction mixture as described, for example, in J. Am. Chem. Soc. 1992, 114, 5959. The compounds of formula (III) thus obtained are converted to compounds of formula (I) as discussed in Scheme 1.

Compounds of formula (Vc), wherein R¹, R², R³, R⁴, R⁵, R⁶, Q, B¹ and B² are as defined for the compounds of formula (I) and wherein R⁰¹ is as described above, may be formed by a Pictet-Spengler reaction between an aldehyde (including formaldehyde in its various forms) of formula (XIII), wherein R⁴ is as defined for the compounds of formula (I), and a compound of formula (XII), wherein R¹, R², R³, R⁵, R⁶, Q, B¹ and B² are as defined for the compounds of formula (I) and wherein R⁰¹ is as described above, by combination with an acid in a suitable solvent, for example as described in Tetrahedron 1987, 43, 439 (Scheme 10).

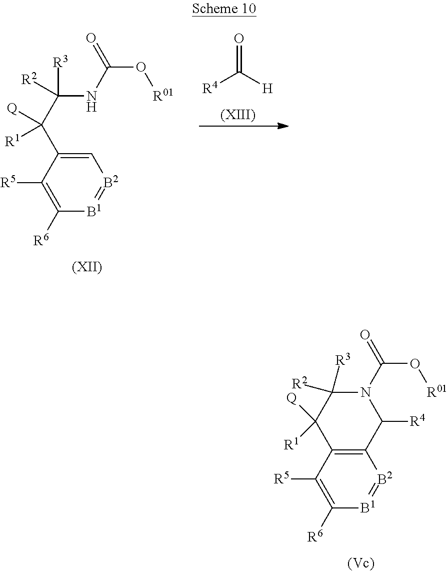

Compounds of formula (XII), wherein R¹, R², R³, R⁵, R⁶, Q, B¹ and B² are as defined for the compounds of formula (I) and wherein R⁰¹ is as described above, may be prepared by a reaction between amines of formula (XIII), wherein R¹, R², R³, R⁵, R⁶, Q, B¹ and B² are as defined for the compounds of formula (I), and a suitable protecting reagent such as methyl chloroformate, optionally in the presence of a base such as triethylamine or pyridine, in a suitable solvent such as DCM at temperatures between −20° C. and the boiling point of the mixture, as for example described in Org. Biomol. Chem. 2016, 14, 6853. This reaction is shown in Scheme 11.

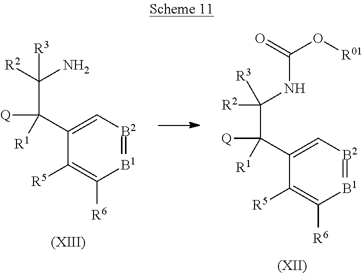

Compounds of formula (XIII), or salts thereof, wherein R¹, R², R³, R⁵, R⁶, Q, B¹ and B² are as defined for the compounds of formula (I), may be prepared by a reaction between nitriles of formula (XIV), wherein R¹, R², R³, R⁴, Q, B¹ and B² are as defined for the compounds of formula (I), and a suitable nucleophile such as (dimethyl sulfide)dihydroboron (BMS) in a suitable aprotic solvent such as THF, for example as described in J. Org Chem. 1981, 47, 3153. Such reactions of nitriles of formula (XIV) leads to compounds of formula (XIII), wherein R² and R³ are hydrogen. Alternatively, Grignard reagents R⁵MgBr or R⁶MgBr, wherein R⁵ and R⁶ are as defined for the compounds of formula (I), may be added as nucleophiles to compounds of formula (XIV), sequentially or simultaneously, to allow more highly substituted amines of formula (XIII) to be prepared. Such Grignard additions to nitriles are carried out in an inert solvent such as diethyl ether, tert-butylmethyl ether, and cyclopentyl methyl ether in the presence of a Lewis acid such as Ti(O-ⁱPr)₄ (see Synlett 2007, (4), 652-654). This reaction is shown in Scheme 12.

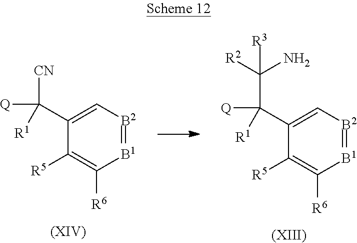

Alternatively compounds of formula (XIII), wherein R¹, R⁵, R⁶, Q, B¹ and B² are as defined for the compounds of formula (I), and wherein R², R³ are hydrogen may be prepared by a person skilled in the art by a reaction between unsaturated nitro compounds of formula (XV), wherein R¹, R⁵, R⁶, B¹ and B² are as defined for the compounds of formula (I), and Grignard reagents of formula (XVI), wherein Q is as defined for the compounds of formula (I) and X⁰ is halogen, preferably iodine, followed by subsequent reduction of compounds of formula (XVII), wherein R¹, R⁵, R⁶, Q, B¹ and B² are as defined for the compounds of formula (I), to compounds formula (XIIIa). This reaction is shown in Scheme 13.

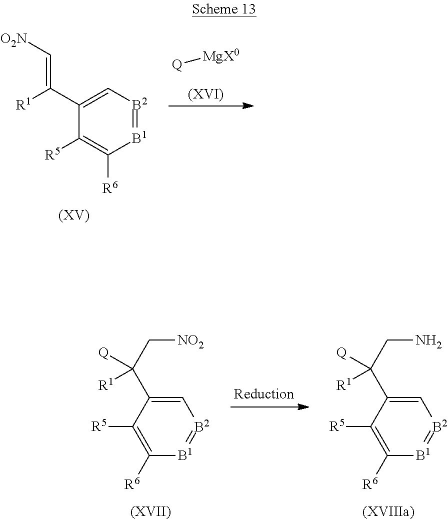

Such conjugate additions of heteroaryl Griganrd reagents to α,β-unsaturated nitro olefins have been reported in the literature (see Synthesis, 1988, (5), 379-81), and a large number of methods exist to reduce nitro groups to amines, some examples of which are reported in “Nitro and Related Groups”, J. Chem. Soc. Perkin Trans. I, 2002, 23, 2586.2597. Gringard's of formula (XVI) are readily prepared from compounds of formula (VIII) by methods known to those skilled in the art.

Compounds of formula (XIV), wherein R¹, R², R³, R⁴, R⁶, R⁹, B¹ and B² are as defined for the compounds of formula (I), may be prepared by a person skilled in the art following known methods. More specifically, compounds of formula (XIV), and intermediates thereof, may be prepared from compounds of formula (XVII) as shown in Scheme 14.

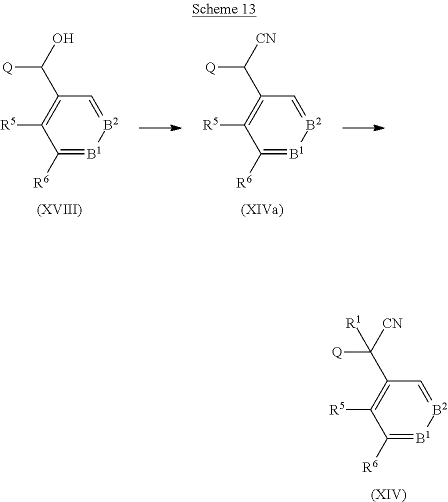

For example, compounds of formula (XIV), wherein R¹, R², R³, R⁸, Q, B¹ and B² are as defined for the compounds of formula (I) and R¹ is different from hydrogen, may be prepared by a person skilled in the art by deprotonation of compound of formula (XIVa) wherein R¹ is hydrogen, and R⁵, R⁶, Q, B¹ and B² are as defined for the compounds of formula (I), using a strong base such as n-butyl lithium or sodium hydride at cryogenic temperatures in an inert solvent such as THF, followed by addition of a suitable alkylating agent R¹—X⁰, wherein R¹ is C₁-C₄alkyl and X⁰ is halogen, for example iodomethane.

Compounds of formula (XIVa), wherein R⁵, R⁶, Q, B¹ and B² are as defined for the compounds of formula (I), may be prepared from alcohols of formula (XVIII) by treatment with cyanotrimethylsilane (TMSCN) in the presence of a base such as lithium carbonate in a nonpolar solvent such as DCM at temperatures between 0° C. and the boiling point of the reaction mixture. Such transformations are well known in the literature under a variety of conditions, for example as described in Org. Lett. 2008, 10, 4570 and references therein. Compounds of formula (XVIII) are readily prepared by Grignard reactions between compounds of formula (XVI) aldehydes of formula (XIX), or Grignard compounds of formula (XXI) with aldehydes of formula (XXII), usually on ether type solvents such as THF, 2.Me-THF, TBME, diethyl ether and the like. Such reactions are well known to those skilled in the art. This is summarized in scheme 15

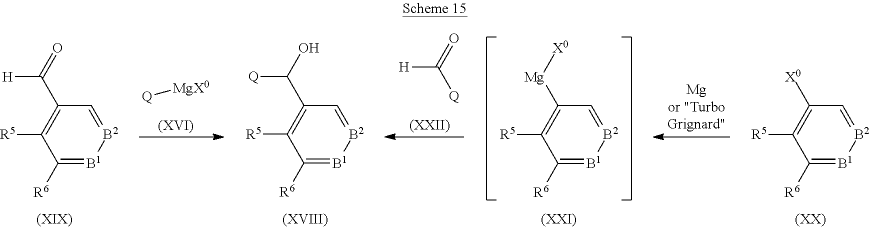

Compounds of formula (II), wherein A¹ is N, A² is O, A⁴ is CH, A³ is C and Z¹ is as defined for the compounds of formula (I), namely compounds of formula (IId) can be prepared as shown in Scheme 16.

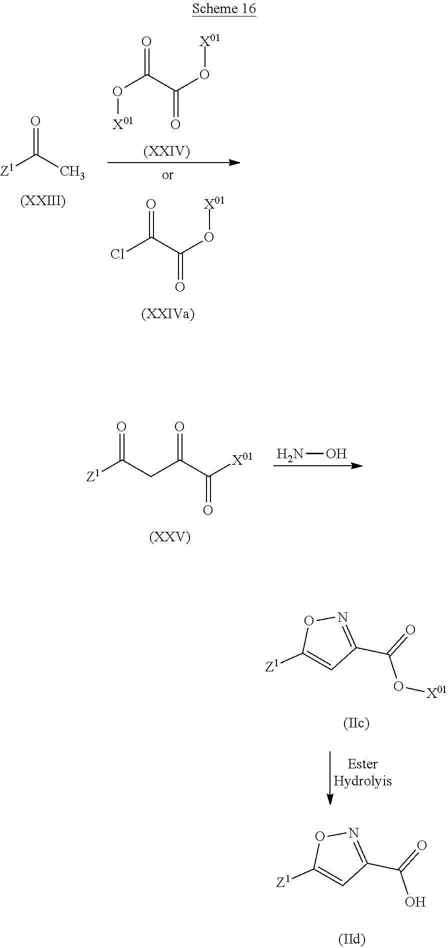

As shown in Scheme 16, compounds of formula (IId) wherein Z¹ is as defined for the compounds of formula (I), and X⁰¹ is C₁-C₄ alkyl may be prepared by hydrolysis of compounds of formula (IIc) by treatment with, for example, an alkaline earth metal hydroxide in water, or with a water miscible organic solvent, such as THF, methanol, ethanol and the like. Such ester hydrolyses are well known to those skilled in the art. Compounds of formula (IIc) can be obtained by the treatment of compounds of formula (XXV) wherein Z¹ is as defined for the compounds of formula (I) and X⁰¹ is C₁-C₄alkyl with hydroxylamine hydrochloride in a polar solvent, for example ethanol and optionally in the presence of a base, e.g. triethyl amine, K₂CO₃ and the like.

Compounds of formula (XXV) are prepared by reaction of compounds of formula (XXIII), wherein Z¹ is as defined for the compounds of formula (I), with compounds of formula (XXIV) or compounds of formula (XXIVa), wherein X⁰¹ is C₁-C₄ alkyl in presence of base such potassium tert-butoxide, sodium hydride or lithium bis(trimethylsilyl)amid in a solvent such THE or toluene. Similar reaction sequences to those described in Scheme 15 to prepare compounds of formula (IIc) and (IId) have been described in for example CN111072582 and WO2019/195810, and WO2018/019929. Further reaction conditions for preparation of compounds of formula (XXV) are described in, for example, Bioorg. Med. Chem. 2016, 109, 350-359.

Compounds of formula (II) wherein A¹ is O, A² and A⁴ are N, A³ is C, and Z¹ is as defined for the compounds of formula (I), namely compounds of formula (IIf) can be prepared as shown in Scheme 17.

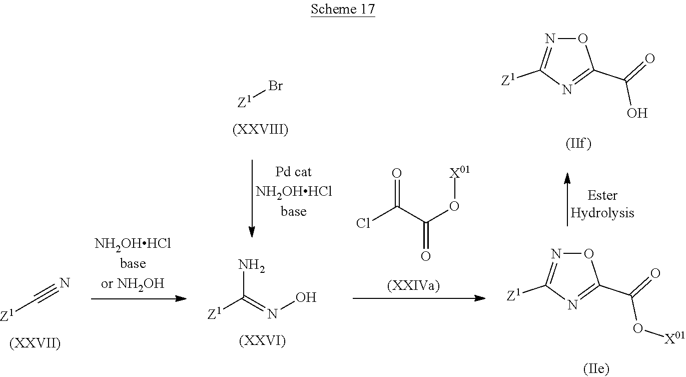

As shown in Scheme 17, compounds of formula (IIf) are obtained by ester hydrolysis of compounds of formula (IIe) as described previously in Scheme 15. Compounds of formula (IIe), wherein Z¹ is as defined for the compounds of formula (I) and X⁰¹ is C₁-C₄ alkyl are prepared by reaction of compounds of formula (XXIVa) with compounds of formula (XXVI), optionally in the presence of base, for example pyridine or triethylamine, in a solvent such as acetonitrile, chloroform or THF. Similar reactions have been reported in for example Bioorg. Med. Chem. 2016, 24(22), 5693-5701 and CN114933573. Compounds of formula (XXVI) wherein Z¹ is as defined for the compounds of formula (I), can be obtained by treatment of compounds of formula (XXVII) with hydroxylamine hydrochloride in presence of base such K₂CO₃ or Na₂CO₃ in a polar solvent for example ethanol. The reaction can also be performed without base using a solution of hydroxylamine. Such reactions have been described in, for example, Bioorg. Med. Chem. Lett. 2020, 30(21), 127508 and Bioorg. Med. Chem. Lett. 2016, 26(23), 5679-5684.

Compounds of formula (XXVI) wherein Z¹ is as defined for the compounds of formula (I), may be also prepared by “one-pot” synthesis via Pd-catalyzed cyanation and amidoximation of compound of formula (XXVIII) wherein Z¹ is as defined for the compounds of formula (I), using potassium ferrocyanide trihydrate and hydroxylamine hydrochloride as described in for example Org. Biomol. Chem. 2015, 13(9), 2541-2545.

Compounds of formula (II) wherein A¹ and A² are N, and A⁴ is O, A³ is C, and Z¹ is as previously described, (namely compounds of formula (IIh), and A¹ and A² are N, and A⁴ is S, A³ is C, and Z¹ is as defined for the compounds of formula (I), (namely compounds of formula (IIj) can be prepared as shown in Scheme 18.

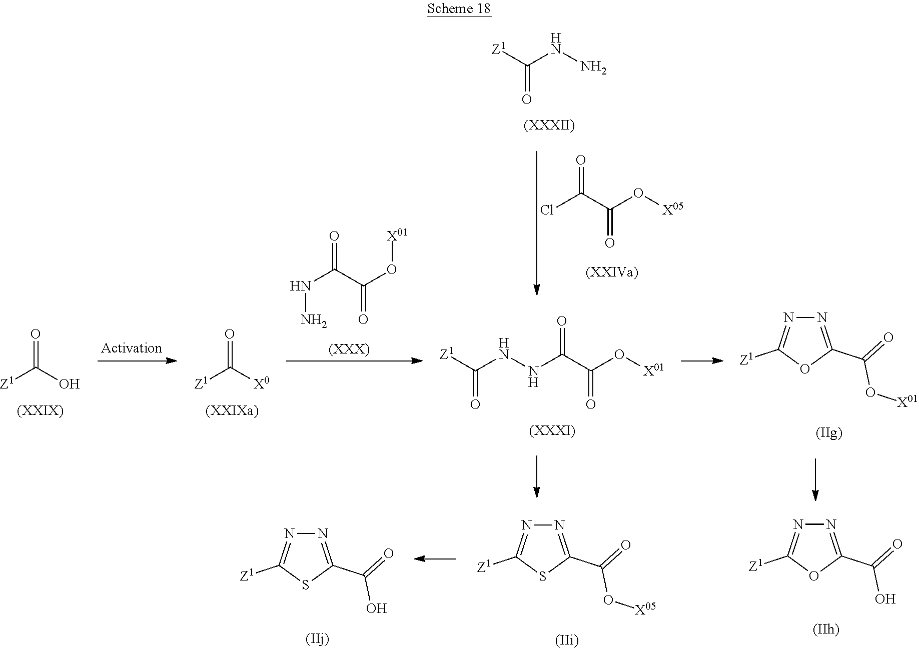

As shown in Scheme 18, compounds of formula (IIh) and (IIj) are obtained by ester hydrolysis of (IIg) and (IIi), respectively. In the latter compounds, X⁰¹ and Z¹ are as previously described. Compounds of formula (IIg) are obtained by dehydration of compounds of formula (XXXI). Compounds of formula (XXXI) are obtained by acylation of hydrazides of formula (XXXII) with compounds of formula (XXIVa). Such sequences of reactions to produce oxadiazoles is well known to those skilled in the art. Similar reactions are described in Bioorg. Med. Chem. Lett. 2005, 15, 1423-1428 and WO2006/044617. Compounds of formula (XXXI) can also be prepared by reaction of activated carboxylic acids of formula (XXIXa), wherein Z¹ is as defined for the compounds of formula (I), and X⁰ are as described in Scheme 1 and Scheme 2 respectively, with compounds of formula (XXX). Compounds of formula (XXIXa) can be prepared from the corresponding acids of formula (XXIX) as described in scheme 1. Such reactions are described for example in, for example, J. Prakt. Chem. 1985, 327, 109-116. Compounds of formula (IIi) can also be prepared from the common intermediate of formula (XXXI), wherein Z¹ is as defined for the compounds of formula (I), and X⁰¹ is as previously described, by treatment with Lawesson's reagent or phosphorous pentasulfide neat, in inert solvents such as toluene or xylene. Similar reactions are known in the literature (see for example WO2010/006713, WO2009/149858 and J. Org Chem. 1961, 26, 4410-12).

Compounds of formula (II) wherein A¹ is O, A² is N, A⁴ is methine, A³ is C, and Z¹ is as defined for the compounds of formula (I), namely compounds of formula (III), can be prepared, for example, as shown in Scheme 19.

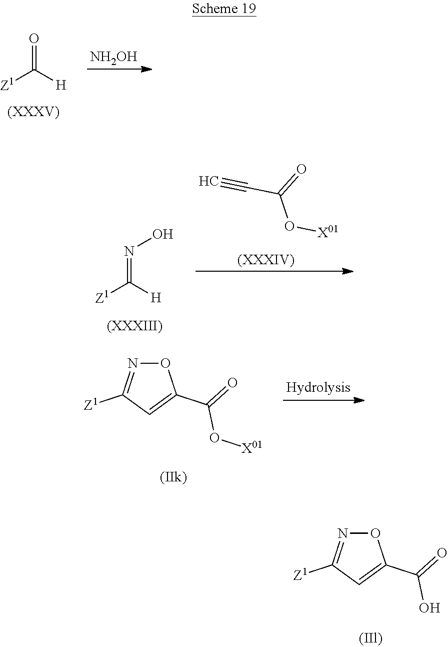

As shown in Scheme 19, compounds of formula (III) are readily obtained by hydrolysis of esters of formula (IIk) by methods known to those skilled in the art and described vide supra. Compounds of formula (IIj) can be obtained by reaction of compounds of formula (XXXIII) with compounds of formula (XXXIV) in the presence of an oxidizing agent, for example (diacetoxyiodo)benzene or N-chlorosuccinimide, in an inert solvent such as methanol or DMF respectively. Such reaction sequences have been described. for example, in J. Het. Chem. 2013, 50(4), 774-780 and J. Chin. Chem. Soc. 2007, 54(3), 643-652. Compounds of formula (XXXIII) are readily prepared from compounds of formula (XXXV), wherein Z¹ is as defined for the compounds of formula (I), by treatment with hydroxylamine under conditions well known to those skilled in the art.

Compounds of formula (II) wherein A¹, A², A⁴ are CH or N, and A³ is N, can be prepared by reaction of compounds of formula (XXXVI)

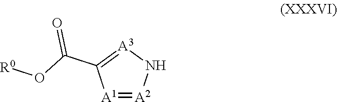

with compounds of formula (XXXVII)

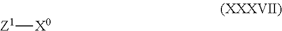

wherein Z¹ is as previously described under formula I and X⁰ is halogen, preferably chlorine, bromine or iodine in the presence of a base, for example and alkaline earth metal base such as NaOH, KOH, LiOH, Cs₂CO₃, K₂CO₃ and the like, in inert aprotic or protic solvents. Such alkylation's are well known to those skilled in the art and have been used in this context to prepare compounds of formula (II) as described for example in WO14/168221, 2013; WO10/043000, 2010; and WO14/32498, 2014. Those skilled in the art will relize this can lead to mixtures of regiosomeric compounds that can be separated by chromatographic techniques, or pure isomers can be obtained by the judicious choice of conditions and additives (for example palladium catalysts for so called Buchwald aminations). For cases where Z¹ is heteroaryl or aryl, SnAr reactions (with or without copper catalysis) may be used to prepare compounds of formula (IIb) (see for example, Polyhedron 2019, 165, 22-30; U.S. Ser. No. 18/017,0909, 2018; Org. Lett. 2022, 24(20), 3620-3625, J. Org. Chem. 2017, 82(14), 7420-7427, Chem. Comm. 2021, 57(57), 7047-7050, ACS Catalysis 2019, 9(12), 10674-10679, Synthesis 2017, 49(23), 5120-5130, J. Org. Chem. 2019, 84(12), 8160-8167, and references cited therein).

Salts of compounds of formula (I) may be prepared in a manner known per se. Thus, for example, acid addition salts of compounds of formula (I) are obtained by treatment with a suitable acid or a suitable ion exchanger reagent and salts with bases are obtained by treatment with a suitable base or with a suitable ion exchanger reagent.

Salts of compounds of formula (I) can be converted in the customary manner into the free compounds (I), acid addition salts, for example, by treatment with a suitable basic compound or with a suitable ion exchanger reagent and salts with bases, for example, by treatment with a suitable acid or with a suitable ion exchanger reagent.

Salts of compounds of formula (I) can be converted in a manner known per se into other salts of compounds of formula (I), acid addition salts, for example, into other acid addition salts, for example by treatment of a salt of inorganic acid such as hydrochloride with a suitable metal salt such as a sodium, barium or silver salt, of an acid, for example with silver acetate, in a suitable solvent in which an inorganic salt which forms, for example silver chloride, is insoluble and thus precipitates from the reaction mixture.

Depending on the procedure or the reaction conditions, the compounds of formula (I), which have salt-forming properties, can be obtained in free form or in the form of salts.

The compounds of formula (I) and, where appropriate, the tautomer's thereof, in each case in free form or in salt form, can be present in the form of one of the isomers which are possible or as a mixture of these, for example in the form of pure isomers, such as antipodes and/or diastereomers, or as isomer mixtures, such as enantiomer mixtures, for example racemates, or diastereomer mixtures, depending on the number, absolute and relative configuration of asymmetric carbon atoms which occur in the molecule and/or depending on the configuration of non-aromatic double bonds which occur in the molecule, the invention relates to the pure isomers and also to all isomer mixtures which are possible and is to be understood in each case in this sense hereinabove and herein below, even when stereochemical details are not mentioned specifically in each case.

Diastereomeric mixtures or racemic mixtures of compounds of formula (I), in free form or in salt form, which can be obtained depending on which starting materials and procedures have been chosen can be separated in a known manner into the pure diastereomers or racemates on the basis of the physicochemical differences of the components, for example by fractional crystallization, distillation and/or chromatography.

Enantiomeric mixtures, such as racemates, which can be obtained in a similar manner can be resolved into the optical antipodes by known methods, for example by recrystallization from an optically active solvent, by chromatography on chiral adsorbents, for example high-performance liquid chromatography (HPLC) on acetyl cellulose, with the aid of suitable microorganisms, by cleavage with specific, immobilized enzymes, via the formation of inclusion compounds, for example using chiral crown ethers, where only one enantiomer is com-plexed, or by conversion into diastereomeric salts, for example by reacting a basic end-product racemate with an optically active acid, such as a carboxylic acid, for example camphor, tartaric or malic acid, or sulfonic acid, for example camphorsulfonic acid, and separating the diastereomer mixture which can be obtained in this manner, for example by fractional crystallization based on their differing solubilities, to give the diastereomers, from which the desired enantiomer can be set free by the action of suitable agents, for example basic agents.

Pure diastereomers or enantiomers can be obtained according to the invention not only by separating suitable isomer mixtures, but also by generally known methods of diastereoselective or enantioselective synthesis, for example by carrying out the process according to the invention with starting materials of a suitable stereochemistry.

It is advantageous to isolate or synthesize in each case the biologically more effective isomer, for example enantiomer or diastereomer, or isomer mixture, for example enantiomer mixture or diastereomer mixture, if the individual components have a different biological activity.

As an example, compounds with more than one asymmetric carbon atoms may exist in diastereomeric forms which can be optionally separated using for example supercritical fluid chromatography (SFC) chromatography with chiral columns. Such diastereomers can show a different fungicidal activity profile, but all isomers and diastereomers form part of this invention.

The compounds of formula (I) have at least three chiral carbon atoms, (three stereocenters, wherein the star (*) indicates the chiral carbon atom), such there are at least eight stereoisomers available. These at least eight stereoisomers consist of four sets of enantiomers.

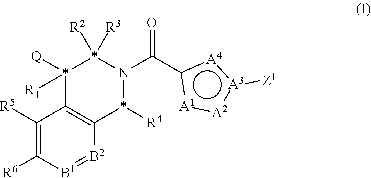

For compounds of formula (I), wherein R¹, R², R³, R⁴, R⁵, R⁶, B¹, B², A¹, A², A³, A⁴, Q and Z¹ are as defined for the compounds of formula (I) and wherein R⁴ is not hydrogen, the relationship between enantiomers and diastereomers is illustrated in Scheme 20.

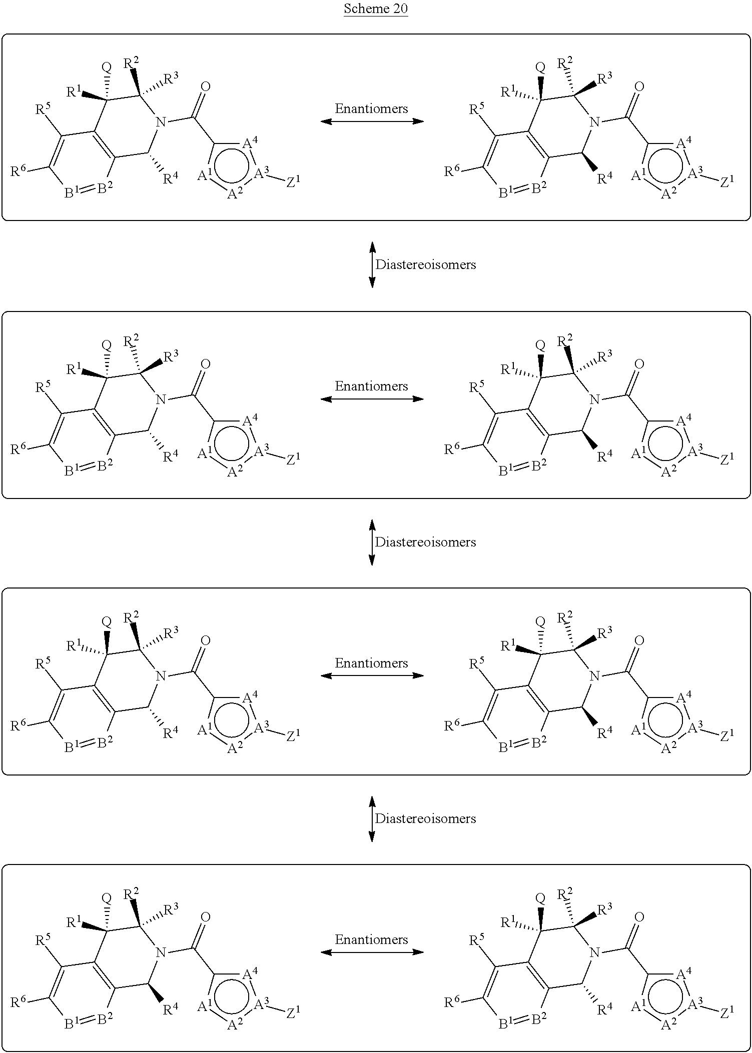

A person skilled in the art is well aware that these diastereomeres and enantiomers of formula (I) (as shown in Scheme 21) wherein Q, R¹, R², R³, R⁴, R⁵, R⁶, B¹, B², A¹, A², A³, A⁴ and Z¹ are as defined for formula (I) and wherein R⁴ is not hydrogen, are within the scope if the invention.

For compounds of formula (I), wherein R², R³ are hydrogen and Q, R¹, R⁴, R⁵, R⁶, B¹, B², and Z¹ are as defined for the compounds of formula (I), and wherein A is defined as for compounds of formula (I-A) and wherein R⁴ is not hydrogen, the relationship between enantiomers and diastereomers is illustrated in Scheme 21.

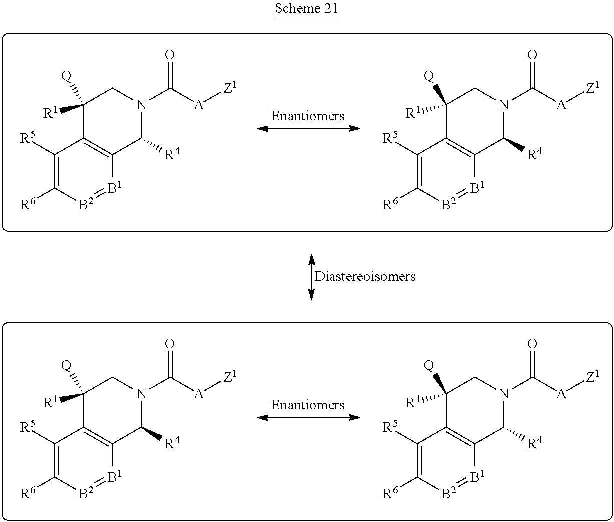

In one embodiment the compounds of formula (I), wherein R², R³ are hydrogen and Q, R¹, R⁴, R⁵, R⁶, B¹, B², and Z¹ are as defined for the compounds of formula (I), and wherein A is defined as for compounds of formula (I-A) and wherein R⁴ is not hydrogen, Q and R⁴ have a syn-relationship to each other.

The syn-Isomers of the compounds of formula (I), wherein R², R³ are hydrogen and Q, R¹, R⁴, R⁵, R⁶, B¹, B², and Z¹ are as defined for the compounds of formula (I), and wherein A is defined as for compounds of formula (I-A), and wherein R⁴ is not hydrogen are shown in Scheme 22.

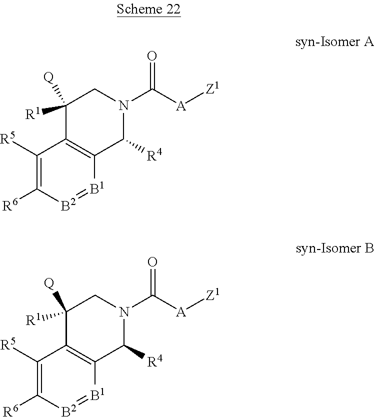

Preferably the compounds of formula (I), wherein R², R³ are hydrogen and Q, R¹, R⁴, R⁵, R⁶, B¹, B², and Z¹ are as defined for the compounds of formula (I), and wherein A is defined as for compounds of formula (I-A) and wherein R⁴ is not hydrogen, Q and R⁴ have a syn-relationship to each other, as shown in Scheme 22.

The compounds of formula (I) and, where appropriate, the tautomers thereof, in each case in free form or in salt form, can, if appropriate, also be obtained in the form of hydrates and/or include other solvents, for example those which may have been used for the crystallization of compounds which are present in solid form.

As already indicated, surprisingly, it has now been found that the compounds of formula (I) of the present invention have, for practical purposes, a very advantageous level of biological activity for protecting plants against diseases that are caused by fungi.

The compounds of formula (I) according to the invention can be used in the agricultural sector and related fields of use, e.g., as active ingredients for controlling plant pests or on non-living materials for the control of spoilage microorganisms or organisms potentially harmful to man. The novel compounds are distinguished by excellent activity at low rates of application, by being well tolerated by plants and by being environmentally safe. They have very useful curative, preventive and systemic properties and can be used for protecting numerous cultivated plants. The compounds of formula (I) can be used to inhibit or destroy the pests that occur on plants or parts of plants (fruit, blossoms, leaves, stems, tubers, roots) of different crops of useful plants, while at the same time protecting also those parts of the plants that grow later, e.g., from phytopathogenic microorganisms.

The present invention further relates to a method for controlling or preventing infestation of plants or plant propagation material and/or harvested food crops susceptible to microbial attack by treating plants or plant propagation material and/or harvested food crops wherein an effective amount a compound of formula (I) according to the invention is applied to the plants, to parts thereof or the locus thereof.

It is also possible to use a compound of formula (I) according to the invention as a fungicide. The term “fungicide” as used herein means a compound that controls, modifies, or prevents the growth of fungi. The term “fungicidally effective amount” where used means the quantity of such a compound or combination of such compounds that is capable of producing an effect on the growth of fungi. Controlling or modifying effects include all deviation from natural development, such as killing, retardation and the like, and prevention includes barrier or other defensive formation in or on a plant to prevent fungal infection.

It may also be possible to use compounds of formula (I) according to the invention as dressing agents for the treatment of plant propagation material, e.g., seed, such as fruits, tubers or grains, or plant cuttings, for the protection against fungal infections as well as against phytopathogenic fungi occurring in the soil. The propagation material can be treated with a composition comprising a compound of formula (I) before planting: seed, for example, can be dressed before being sown. The active compounds of formula (I) can also be applied to grains (coating), either by impregnating the seeds in a liquid formulation or by coating them with a solid formulation. The composition can also be applied to the planting site when the propagation material is being planted, for example, to the seed furrow during sowing. The invention relates also to such methods of treating plant propagation material and to the plant propagation material so treated.

Furthermore, the compounds of formula (I) according to the invention can be used for controlling fungi in related areas, for example in the protection of technical materials, including wood and wood related technical products, in food storage, in hygiene management.

In addition, the invention could be used to protect non-living materials from fungal attack, e.g. lumber, wall boards and paint.

The compounds of formula (I) according to the invention are for example, effective against fungi and fungal vectors of disease as well as phytopathogenic bacteria and viruses. These fungi and fungal vectors of disease as well as phytopathogenic bacteria and viruses are for example: Absidia corymbifera, Alternaria spp., Aphanomyces spp., Ascochyta spp., Aspergillus spp. including A. flavus, A. fumigatus, A. nidulans, A. niger, A. terrus, Aureobasidium spp. including A. pullulans, Blastomyces dermatitidis, Blumeria graminis, Bremia lactucae, Botryosphaeria spp. including B. dothidea, B. obtusa, Botrytis spp. including B. cinerea, Candida spp. including C. albicans, C. glabrata, C. krusei, C. lusitaniae, C. parapsilosis, C. tropicalis, Cephaloascus fragrans, Ceratocystis spp., Cercospora spp. including C. arachidicola, Cercosporidium personatum, Cladosporium spp., Claviceps purpurea, Coccidioides immitis, Cochliobolus spp., Colletotrichum spp. including C. musae, Cryptococcus neoformans, Diaporthe spp., Didymella spp., Drechslera spp., Elsinoe spp., Epidermophyton spp., Erwinia amylovora, Erysiphe spp. including E. cichoracearum, Eutypa lata, Fusarium spp. including F. culmorum, F. graminearum, F. langsethiae, F. moniliforme, F. oxysporum, F. proliferatum, F. subglutinans, F. solani, Gaeumannomyces graminis, Gibberella fujikuroi, Gloeodes pomigena, Gloeosporium musarum, Glomerella cingulate, Guignardia bidwellii, Gymnosporangium juniperi-virginianae, Helminthosporium spp., Hemileia spp., Histoplasma spp. including H. capsulatum, Laetisaria fuciformis, Leptographium lindbergi, Leveillula taurica, Lophodermium seditiosum, Microdochium nivale, Microsporum spp., Monilinia spp., Mucor spp., Mycosphaerella spp. including M. graminicola, M. pomi, Oncobasidium theobromaeon, Ophiostoma piceae, Paracoccidioides spp., Penicillium spp. including P. digitatum, P. italicum, Petriellidium spp., Peronosclerospora spp. Including P. maydis, P. philippinensis and P. sorghi, Peronospora spp., Phaeosphaeria nodorum, Phakopsora pachyrhizi, Phellinus igniarus, Phialophora spp., Phoma spp., Phomopsis viticola, Phytophthora spp. including P. infestans, Plasmopara spp. including P. halstedii, P. viticola, Pleospora spp., Podosphaera spp. including P. leucotricha, Polymyxa graminis, Polymyxa betae, Pseudocercosporella herpotrichoides, Pseudomonas spp., Pseudoperonospora spp. including P. cubensis, P. humuli, Pseudopeziza tracheiphila, Puccinia spp. including P. hordei, P. recondita, P. striiformis, P. triticina, Pyrenopeziza spp., Pyrenophora spp., Pyricularia spp. including P. oryzae, Pythium spp. including P. ultimum, Ramularia spp., Rhizoctonia spp., Rhizomucor pusillus, Rhizopus arrhizus, Rhynchosporium spp., Scedosporium spp. including S. apiospermum and S. prolificans, Schizothyrium pomi, Sclerotinia spp., Sclerotium spp., Septoria spp., including S. nodorum, S. tritici, Sphaerotheca macularis, Sphaerotheca fusca (Sphaerotheca fuliginea), Sporothorix spp., Stagonospora nodorum, Stemphylium spp., Stereum hirsutum, Thanatephorus cucumeris, Thielaviopsis basicola, Tilletia spp., Trichoderma spp. including T. harzianum, T. pseudokoningii, T. viride, Trichophyton spp., Typhula spp., Uncinula necator, Urocystis spp., Ustilago spp., Venturia spp. including V. inaequalis, Verticillium spp., and Xanthomonas spp.

The compounds of formula (I) according to the invention may be used for example on turf, ornamentals, such as flowers, shrubs, broad-leaved trees, or evergreens, for example conifers, as well as for tree injection, pest management and the like.

Within the scope of present invention, target crops and/or useful plants to be protected typically comprise perennial and annual crops, such as berry plants for example blackberries, blueberries, cranberries, raspberries and strawberries; cereals for example barley, maize (corn), millet, oats, rice, rye, sorghum triticale and wheat; fibre plants for example cotton, flax, hemp, jute and sisal; field crops for example sugar and fodder beet, coffee, hops, mustard, oilseed rape (canola), poppy, sugar cane, sunflower, tea and tobacco; fruit trees for example apple, apricot, avocado, banana, cherry, citrus, nectarine, peach, pear and plum; grasses for example Bermuda grass, bluegrass, bentgrass, centipede grass, fescue, ryegrass, St. Augustine grass and Zoysia grass; herbs such as basil, borage, chives, coriander, lavender, lovage, mint, oregano, parsley, rosemary, sage and thyme; legumes for example beans, lentils, peas and soya beans; nuts for example almond, cashew, ground nut, hazelnut, peanut, pecan, pistachio and walnut; palms for example oil palm; ornamentals for example flowers, shrubs and trees; other trees, for example cacao, coconut, olive and rubber; vegetables for example asparagus, aubergine, broccoli, cabbage, carrot, cucumber, garlic, lettuce, marrow, melon, okra, onion, pepper, potato, pumpkin, rhubarb, spinach and tomato; and vines for example grapes.

The term “useful plants” is to be understood as also including useful plants that have been rendered tolerant to herbicides like bromoxynil or classes of herbicides (such as, for example, HPPD inhibitors, ALS inhibitors, for example primisulfuron, prosulfuron and trifloxysulfuron, EPSPS (5-enol-pyrovyl-shikimate-3-phosphate-synthase) inhibitors, GS (glutamine synthetase) inhibitors or PPO (protoporphyrinogen-oxidase) inhibitors) as a result of conventional methods of breeding or genetic engineering. An example of a crop that has been rendered tolerant to imidazolinones, e.g. imazamox, by conventional methods of breeding (mutagenesis) is Clearfield® summer rape (Canola). Examples of crops that have been rendered tolerant to herbicides or classes of herbicides by genetic engineering methods include glyphosate- and glufosinate-resistant maize varieties commercially available under the trade names RoundupReady®, Herculex I® and LibertyLink®.

The term “useful plants” is to be understood as also including useful plants which have been so transformed by the use of recombinant DNA techniques that they are capable of synthesising one or more selectively acting toxins, such as are known, for example, from toxin-producing bacteria, especially those of the genus Bacillus.

Examples of such plants are: YieldGard® (maize variety that expresses a CrylA(b) toxin); YieldGard Rootworm® (maize variety that expresses a CrylIIB(b1) toxin); YieldGard Plus® (maize variety that expresses a CrylA(b) and a CrylIIB(b1) toxin); Starlink® (maize variety that expresses a Cry9(c) toxin); Herculex I® (maize variety that expresses a CrylF(a2) toxin and the enzyme phosphinothricine N-acetyltransferase (PAT) to achieve tolerance to the herbicide glufosinate ammonium); NuCOTN 33B® (cotton variety that expresses a CrylA(c) toxin); Bollgard I® (cotton variety that expresses a CrylA(c) toxin); Bollgard II® (cotton variety that expresses a CrylA(c) and a CrylIA(b) toxin); VIPCOT® (cotton variety that expresses a VIP toxin); NewLeaf® (potato variety that expresses a CrylIIA toxin); Nature-Gard® Agrisure® GT Advantage (GA21 glyphosate-tolerant trait), Agrisure® CB Advantage (Bt11 corn borer (CB) trait), Agrisure® RW (corn rootworm trait) and Protecta®.

The term “crops” is to be understood as including also crop plants which have been so transformed using recombinant DNA techniques that they are capable of synthesising one or more selectively acting toxins, such as are known, for example, from toxin-producing bacteria, especially those of the genus Bacillus.

Toxins that can be expressed by such transgenic plants include, for example, insecticidal proteins from Bacillus cereus or Bacillus popilliae; or insecticidal proteins from Bacillus thuringiensis, such as delta-endotoxins, e.g., Cry1Ab, Cry1Ac, Cry1F, CrylFa2, Cry2Ab, Cry3A, Cry3Bb1 or Cry9C, or vegetative insecticidal proteins (Vip), e.g. Vip1, Vip2, Vip3 or Vip3A; or insecticidal proteins of bacteria colonising nematodes, for example Photorhabdus spp. or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as Streptomycetes toxins, plant lectins, such as pea lectins, barley lectins or snowdrop lectins; agglutinins; proteinase inhibitors, such as trypsin inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases.

Further, in the context of the present invention there are to be understood by delta-endotoxins, for example Cry1Ab, Cry1Ac, Cry1F, CrylFa2, Cry2Ab, Cry3A, Cry3Bb1 or Cry9C, or vegetative insecticidal proteins (Vip), for example Vip1, Vip2, Vip3 or Vip3A, expressly also hybrid toxins, truncated toxins and modified toxins. Hybrid toxins are produced recombinantly by a new combination of different domains of those proteins (see, for example, WO02/15701). Truncated toxins, for example a truncated Cry1Ab, are known. In the case of modified toxins, one or more amino acids of the naturally occurring toxin are replaced. In such amino acid replacements, preferably non-naturally present protease recognition sequences are inserted into the toxin, such as, for example, in the case of Cry3A055, a cathepsin-G-recognition sequence is inserted into a Cry3A toxin (see WO2003/018810).

Examples of such toxins or transgenic plants capable of synthesising such toxins are disclosed, for example, in EP-0374753, WO93/07278, WO95/34656, EP0427529, EP0451878 and WO03/052073.

The processes for the preparation of such transgenic plants are generally known to a person skilled in the art and are described, for example, in the publications mentioned above. Cryl-type deoxyribonucleic acids and their preparation are known, for example, from WO95/34656, EP0367474, EPO401979 and WO90/13651.

The toxin contained in the transgenic plants imparts to the plants tolerance to harmful insects. Such insects can occur in any taxonomic group of insects but are especially commonly found in the beetles (Coleoptera), two-winged insects (Diptera) and butterflies (Lepidoptera).

Transgenic plants containing one or more genes that code for an insecticidal resistance and express one or more toxins are known and some of them are commercially available. Examples of such plants are: YieldGard® (maize variety that expresses a Cry1Ab toxin); YieldGard Rootworm® (maize variety that expresses a Cry3Bb1 toxin); YieldGard Plus® (maize variety that expresses a Cry1Ab and a Cry3Bb1 toxin); Starlink® (maize variety that expresses a Cry9C toxin); Herculex I® (maize variety that expresses a Cry1 Fa2 toxin and the enzyme phosphinothricine N-acetyltransferase (PAT) to achieve tolerance to the herbicide glufosinate ammonium); NuCOTN 33B® (cotton variety that expresses a Cry1Ac toxin); Bollgard I® (cotton variety that expresses a Cry1Ac toxin); Bollgard II® (cotton variety that expresses a CrylAc and a Cry2Ab toxin); VipCot® (cotton variety that expresses a Vip3A and a Cry1Ab toxin); NewLeaf® (potato variety that expresses a Cry3A toxin); NatureGard®, Agrisure® GT Advantage (GA21 glyphosate-tolerant trait), Agrisure® CB Advantage (Bt11 corn borer (CB) trait) and Protecta®.

Further examples of such transgenic crops are:
1. Btl1 Maize from Syngenta Seeds SAS, Chemin de I'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a truncated Cry1Ab toxin. Bt11 maize also transgenically expresses the enzyme PAT to achieve tolerance to the herbicide glufosinate ammonium.
2. Bt176 Maize from Syngenta Seeds SAS, Chemin de I'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a Cry1Ab toxin. Bt176 maize also transgenically expresses the enzyme PAT to achieve tolerance to the herbicide glufosinate ammonium.
3. MIR604 Maize from Syngenta Seeds SAS, Chemin de I'Hobit 27, F-31 790 St. Sauveur, France, registration number C/FR/96/05/10. Maize which has been rendered insect-resistant by transgenic expression of a modified Cry3A toxin. This toxin is Cry3A055 modified by insertion of a cathepsin-G-protease recognition sequence. The preparation of such transgenic maize plants is described in WO2003/01881 0.
4. MON 863 Maize from Monsanto Europe S.A. 270-272 Avenue de Tervuren, B-1150 Brussels, Belgium, registration number C/DE/02/9. MON 863 expresses a Cry3Bb1 toxin and has resistance to certain Coleoptera insects.
5. IPC 531 Cotton from Monsanto Europe S.A. 270-272 Avenue de Tervuren, B-1150 Brussels, Belgium, registration number C/ES/96/02.
6. 1507 Maize from Pioneer Overseas Corporation, Avenue Tedesco, 7 B-1160 Brussels, Belgium, registration number C/NL/00/10. Genetically modified maize for the expression of the protein Cry1F for achieving resistance to certain Lepidoptera insects and of the PAT protein for achieving tolerance to the herbicide glufosinate ammonium.
7. NK603×MON 810 Maize from Monsanto Europe S.A. 270-272 Avenue de Tervuren, B 1150 Brussels, Belgium, registration number C/GB/02/M3/03. Consists of conventionally bred hybrid maize varieties by crossing the genetically modified varieties NK603 and MON 810. NK603×MON 810 Maize transgenically expresses the protein CP4 EPSPS, obtained from Agrobacterium sp. strain CP4, which imparts tolerance to the herbicide Roundup® (contains glyphosate), and also a Cry1Ab toxin obtained from Bacillus thuringiensis subsp. kurstaki which brings about tolerance to certain Lepidoptera, include the European corn borer.

The compounds of formula (I) according to the invention may be used in controlling or preventing phytopathogenic diseases, especially phytopathogenic fungi such as Alternaria species in fruits, vegetables and potatoes; Botrytis cinerea in strawberries, tomatoes, sunflower, pulse crops, vegetables and grapes; Rhizoctonia solani in potatoes and vegetables; Uncinula necator in grapes; Cladosporium cucumerinum, Didymella bryoniae, Sphaerotheca fuliginea and Glomerella lagenarium in cucurbits; Leveillula taurica in cucurbits and solanacious crops; Fusarium spp. in cereals; Leptosphaeria spp. in cereals; and Zymospetoria spp. in cereals.

The term “locus” as used herein means fields in or on which plants are growing, or where seeds of cultivated plants are sown, or where seed will be placed into the soil. It includes soil, seeds, and seedlings, as well as established vegetation.

The term “plants” refers to all physical parts of a plant, including seeds, seedlings, saplings, roots, tubers, stems, stalks, foliage, and fruits.

The term “plant propagation material” is understood to denote generative parts of the plant, such as seeds, which can be used for the multiplication of the latter, and vegetative material, such as cuttings or tubers, for example potatoes. There can be mentioned for example seeds (in the strict sense), roots, fruits, tubers, bulbs, rhizomes and parts of plants. Germinated plants and young plants which are to be transplanted after germination or after emergence from the soil, may also be mentioned. These young plants can be protected before transplantation by a total or partial treatment by immersion. Preferably “plant propagation material” is understood to denote seeds.

The compounds of formula (I) according to the invention may be used in unmodified form or, preferably, together with the adjuvants conventionally employed in the art of formulation. To this end they may be conveniently formulated in known manner to emulsifiable concentrates, coatable pastes, directly sprayable or dilutable solutions or suspensions, dilute emulsions, wettable powders, soluble powders, dusts, granulates, and also encapsulations e.g. in polymeric substances. As with the type of the compositions, the methods of application, such as spraying, atomising, dusting, scattering, coating or pouring, are chosen in accordance with the intended objectives and the prevailing circumstances. The compositions may also contain further adjuvants such as stabilizers, antifoams, viscosity regulators, binders or tackifiers as well as fertilizers, micronutrient donors or other formulations for obtaining special effects.

Suitable carriers and adjuvants, e.g. for agricultural use, can be solid or liquid and are substances useful in formulation technology, e.g. natural or regenerated mineral substances, solvents, dispersants, wetting agents, tackifiers, thickeners, binders or fertilizers. Such carriers are for example described in WO1997/33890.

Suspension concentrates are aqueous formulations in which finely divided solid particles of the active compound are suspended. Such formulations include anti-settling agents and dispersing agents and may further include a wetting agent to enhance activity as well an anti-foam and a crystal growth inhibitor. In use, these concentrates are diluted in water and normally applied as a spray to the area to be treated. The amount of active ingredient may range from 0.5% to 95% of the concentrate.

Wettable powders are in the form of finely divided particles which disperse readily in water or other liquid carriers. The particles contain the active ingredient retained in a solid matrix. Typical solid matrices include fuller's earth, kaolin clays, silicas and other readily wet organic or inorganic solids. Wettable powders normally contain from 5% to 95% of the active ingredient plus a small amount of wetting, dispersing or emulsifying agent.

Emulsifiable concentrates are homogeneous liquid compositions dispersible in water or other liquid and may consist entirely of the active compound with a liquid or solid emulsifying agent, or may also contain a liquid carrier, such as xylene, heavy aromatic naphthas, isophorone and other non-volatile organic solvents. In use, these concentrates are dispersed in water or other liquid and normally applied as a spray to the area to be treated. The amount of active ingredient may range from 0.5% to 95% of the concentrate.

Granular formulations include both extrudates and relatively coarse particles and are usually applied without dilution to the area in which treatment is required. Typical carriers for granular formulations include sand, fuller's earth, attapulgite clay, bentonite clays, montmorillonite clay, vermiculite, perlite, calcium carbonate, brick, pumice, pyrophyllite, kaolin, dolomite, plaster, wood flour, ground corn cobs, ground peanut hulls, sugars, sodium chloride, sodium sulphate, sodium silicate, sodium borate, magnesia, mica, iron oxide, zinc oxide, titanium oxide, antimony oxide, cryolite, gypsum, diatomaceous earth, calcium sulphate and other organic or inorganic materials which absorb or which can be coated with the active compound. Granular formulations normally contain 5% to 25% of active ingredients which may include surface-active agents such as heavy aromatic naphthas, kerosene and other petroleum fractions, or vegetable oils; and/or stickers such as dextrins, glue or synthetic resins.

Dusts are free-flowing admixtures of the active ingredient with finely divided solids such as talc, clays, flours and other organic and inorganic solids which act as dispersants and carriers.

Microcapsules are typically droplets or granules of the active ingredient enclosed in an inert porous shell which allows escape of the enclosed material to the surroundings at controlled rates. Encapsulated droplets are typically 1 to 50 microns in diameter. The enclosed liquid typically constitutes 50 to 95% of the weight of the capsule and may include solvent in addition to the active compound. Encapsulated granules are generally porous granules with porous membranes sealing the granule pore openings, retaining the active species in liquid form inside the granule pores. Granules typically range from 1 millimetre to 1 centimetre and preferably 1 to 2 millimetres in diameter. Granules are formed by extrusion, agglomeration or prilling, or are naturally occurring. Examples of such materials are vermiculite, sintered clay, kaolin, attapulgite clay, sawdust and granular carbon. Shell or membrane materials include natural and synthetic rubbers, cellulosic materials, styrene-butadiene copolymers, polyacrylonitriles, polyacrylates, polyesters, polyamides, polyureas, polyurethanes and starch xanthates.

Other useful formulations for agrochemical applications include simple solutions of the active ingredient in a solvent in which it is completely soluble at the desired concentration, such as acetone, alkylated naphthalenes, xylene and other organic solvents. Pressurised sprayers, wherein the active ingredient is dispersed in finely divided form as a result of vaporisation of a low boiling dispersant solvent carrier, may also be used.

Suitable agricultural adjuvants and carriers that are useful in formulating the compositions of the invention in the formulation types described above are well known to a person skilled in the art.

Liquid carriers that can be employed include, for example, water, toluene, xylene, petroleum naphtha, crop oil, acetone, methyl ethyl ketone, cyclohexanone, acetic anhydride, acetonitrile, acetophenone, amyl acetate, 2-butanone, chlorobenzene, cyclohexane, cyclohexanol, alkyl acetates, diacetonalcohol, 1,2-dichloropropane, diethanolamine, p diethylbenzene, diethylene glycol, diethylene glycol abietate, diethylene glycol butyl ether, diethylene glycol ethyl ether, diethylene glycol methyl ether, N,N-dimethyl formamide, dimethyl sulfoxide, 1,4-dioxane, dipropylene glycol, dipropylene glycol methyl ether, dipropylene glycol dibenzoate, diproxitol, alkyl pyrrolidinone, ethyl acetate, 2-ethyl hexanol, ethylene carbonate, 1,1,1-trichloroethane, 2-heptanone, alpha pinene, d-limonene, ethylene glycol, ethylene glycol butyl ether, ethylene glycol methyl ether, gamma-butyrolactone, glycerol, glycerol diacetate, glycerol monoacetate, glycerol triacetate, hexadecane, hexylene glycol, isoamyl acetate, isobornyl acetate, isooctane, isophorone, isopropyl benzene, isopropyl myristate, lactic acid, laurylamine, mesityl oxide, methoxy-propanol, methyl isoamyl ketone, methyl isobutyl ketone, methyl laurate, methyl octanoate, methyl oleate, methylene chloride, m-xylene, n-hexane, n-octylamine, octadecanoic acid, octyl amine acetate, oleic acid, oleylamine, o-xylene, phenol, polyethylene glycol (PEG400), propionic acid, propylene glycol, propylene glycol monomethyl ether, p-xylene, toluene, triethyl phosphate, triethylene glycol, xylene sulfonic acid, paraffin, mineral oil, trichloroethylene, perchloroethylene, ethyl acetate, amyl acetate, butyl acetate, methanol, ethanol, isopropanol, and higher molecular weight alcohols such as amyl alcohol, tetrahydrofurfuryl alcohol, hexanol, octanol, etc., ethylene glycol, propylene glycol, glycerine and N-methyl-2-pyrrolidinone. Water is generally the carrier of choice forthe dilution of concentrates.

Suitable solid carriers include, for example, talc, titanium dioxide, pyrophyllite clay, silica, attapulgite clay, kieselguhr, chalk, diatomaxeous earth, lime, calcium carbonate, bentonite clay, fuller's earth, cotton seed hulls, wheat flour, soybean flour, pumice, wood flour, walnut shell flour and lignin.

A broad range of surface-active agents are advantageously employed in both said liquid and solid compositions, especially those designed to be diluted with carrier before application. These agents, when used, normally comprise from 0.1% to 15% by weight of the formulation. They can be anionic, cationic, non-ionic or polymeric in character and can be employed as emulsifying agents, wetting agents, suspending agents or for other purposes. Typical surface-active agents include salts of alkyl sulfates, such as diethanolammonium lauryl sulphate; alkylarylsulfonate salts, such as calcium dodecylbenzenesulfonate; alkylphenol-alkylene oxide addition products, such as nonylphenol-C.sub. 18 ethoxylate; alcohol-alkylene oxide addition products, such as tridecyl alcohol-C.sub. 16 ethoxylate; soaps, such as sodium stearate; alkylnaphthalenesulfonate salts, such as sodium dibutylnaphthalenesulfonate; dialkyl esters of sulfosuccinate salts, such as sodium di(2 ethylhexyl) sulfosuccinate; sorbitol esters, such as sorbitol oleate; quaternary amines, such as lauryl trimethylammonium chloride; polyethylene glycol esters of fatty acids, such as polyethylene glycol stearate; block copolymers of ethylene oxide and propylene oxide; and salts of mono and dialkyl phosphate esters.

Other adjuvants commonly utilized in agricultural compositions include crystallisation inhibitors, viscosity modifiers, suspending agents, spray droplet modifiers, pigments, antioxidants, foaming agents, anti-foaming agents, light-blocking agents, compatibilizing agents, antifoam agents, sequestering agents, neutralising agents and buffers, corrosion inhibitors, dyes, odorants, spreading agents, penetration aids, micronutrients, emollients, lubricants and sticking agents.

In addition, further, other biocidal active ingredients or compositions may be combined with the compositions of the invention and used in the methods of the invention and applied simultaneously or sequentially with the compositions of the invention. When applied simultaneously, these further active ingredients may be formulated together with the compositions of the invention or mixed in, for example, the spray tank. These further biocidal active ingredients may be fungicides, herbicides, insecticides, bactericides, acaricides, nematicides and/or plant growth regulators.

Pesticidal agents are referred to herein using their common name are known, for example, from “The Pesticide Manual”, 15th Ed., British Crop Protection Council 2009.

In addition, the compositions of the invention may also be applied with one or more systemically acquired resistance inducers (“SAR” inducer). SAR inducers are known and described in, for example, U.S. Pat. No. 6,919,298 and include, for example, salicylates and the commercial SAR inducer acibenzolar-S-methyl.

The compounds of formula (I) according to the invention are normally used in the form of agrochemical compositions and can be applied to the crop area or plant to be treated, simultaneously or in succession with further compounds. These further compounds can be e.g., fertilizers or micronutrient donors or other preparations, which influence the growth of plants. They can also be selective herbicides or non-selective herbicides as well as insecticides, fungicides, bactericides, nematicides, molluscicides or mixtures of several of these preparations, if desired together with further carriers, surfactants or application promoting adjuvants customarily employed in the art of formulation.

The compounds of formula (I) according to the invention may be used in the form of (fungicidal) compositions for controlling or protecting against phytopathogenic microorganisms, comprising as active ingredient at least one compound of formula (I) or of at least one preferred individual compound as defined herein, in free form or in agrochemical usable salt form, and at least one of the above-mentioned adjuvants.

The invention therefore provides a composition, preferably a fungicidal composition, comprising at least one compound of formula (I) according to the invention, an agriculturally acceptable carrier and optionally an adjuvant. An agricultural acceptable carrier is for example a carrier that is suitable for agricultural use.

Agricultural carriers are well known in the art. Preferably, said composition may comprise at least one or more pesticidal-active compounds, for example an additional fungicidal active ingredient in addition to the compound of formula (I).

The compound of formula (I) according to the invention may be the sole active ingredient of a composition or it may be admixed with one or more additional active ingredients such as a pesticide, fungicide, synergist, herbicide or plant growth regulator where appropriate. An additional active ingredient may, in some cases, result in unexpected synergistic activities.

Examples of suitable additional active ingredients include the following: acycloamino acid fungicides, aliphatic nitrogen fungicides, amide fungicides, anilide fungicides, antibiotic fungicides, aromatic fungicides, arsenical fungicides, aryl phenyl ketone fungicides, benzamide fungicides, benzanilide fungicides, benzimidazole fungicides, benzothiazole fungicides, botanical fungicides, bridged diphenyl fungicides, carbamate fungicides, carbanilate fungicides, conazole fungicides, copper fungicides, dicarboximide fungicides, dinitrophenol fungicides, dithiocarbamate fungicides, dithiolane fungicides, furamide fungicides, furanilide fungicides, hydrazide fungicides, imidazole fungicides, mercury fungicides, morpholine fungicides, organophosphorous fungicides, organotin fungicides, oxathiin fungicides, oxazole fungicides, phenylsulfamide fungicides, polysulfide fungicides, pyrazole fungicides, pyridine fungicides, pyrimidine fungicides, pyrrole fungicides, quaternary ammonium fungicides, quinoline fungicides, quinone fungicides, quinoxaline fungicides, strobilurin fungicides, sulfonanilide fungicides, thiadiazole fungicides, thiazole fungicides, thiazolidine fungicides, thiocarbamate fungicides, thiophene fungicides, triazine fungicides, triazole fungicides, triazolopyrimidine fungicides, urea fungicides, valinamide fungicides, and zinc fungicides.

Examples of suitable additional active ingredients include the following: petroleum oils, 1,1-bis(4-chlorophenyl)-2-ethoxyethanol, 2,4-dichlorophenyl benzenesulfonate, 2-fluoro-N-methyl-N-1-naphthylacetamide, 4-chlorophenyl phenyl sulfone, acetoprole, aldoxycarb, amidithion, amidothioate, amiton, amiton hydrogen oxalate, amitraz, aramite, arsenous oxide, azobenzene, azothoate, benomyl, benoxa-fos, benzyl benzoate, bixafen, brofenvalerate, bromocyclen, bromophos, bromopropylate, buprofezin, butocarboxim, butoxycarboxim, butylpyridaben, calcium polysulfide, camphechlor, carbanolate, carbophenothion, cymiazole, chinomethionat, chlorbenside, chlordimeform, chlordimeform hydrochloride, chlorfenethol, chlorfenson, chlorfensulfide, chlorobenzilate, chloromebuform, chloromethiuron, chloropropylate, chlorthiophos, cinerin I, cinerin II, cinerins, closantel, coumaphos, crotamiton, crotoxyphos, cufraneb, cyanthoate, DCPM, DDT, demephion, demephion-O, demephion-S, demeton-methyl, demeton-O, demeton-O-methyl, demeton-S, demeton-S-methyl, demeton-S-methylsulfon, dichlofluanid, dichlorvos, dicliphos, dienochlor, dimefox, dinex, dinex-diclexine, dinocap-4, dinocap-6, dinocton, dinopenton, dinosulfon, dinoterbon, dioxathion, diphenyl sulfone, disulfiram, DNOC, dofenapyn, doramectin, endothion, eprinomectin, ethoate-methyl, etrimfos, fenazaflor, fenbutatin oxide, fenothiocarb, fenpyrad, fenpyroximate, fenpyrazamine, fenson, fentrifanil, flubenzimine, flucycloxuron, fluenetil, fluorbenside, FMC 1137, formetanate, formetanate hydrochloride, formparanate, gamma-HCH, glyodin, halfenprox, hexadecyl cyclopropanecarboxylate, isocarbophos, jasmolin I, jasmolin II, jodfenphos, lindane, malonoben, mecarbam, mephosfolan, mesulfen, methacrifos, methyl bromide, metolcarb, mexacarbate, milbemycin oxime, mipafox, monocrotophos, morphothion, moxidectin, naled, 4-chloro-2-(2-chloro-2-methyl-propyl)-5-[(6-iodo-3-pyridyl)methoxy]pyridazin-3-one, nifluridide, nikkomycins, nitrilacarb, nitrilacarb 1:1 zinc chloride complex, omethoate, oxydeprofos, oxydisulfoton, pp′-DDT, parathion, permethrin, phenkapton, phosalone, phosfolan, phosphamidon, polychloroterpenes, polynactins, proclonol, promacyl, propoxur, prothidathion, prothoate, pyrethrin I, pyrethrin II, pyrethrins, pyridaphenthion, pyrimitate, quinalphos, quintiofos, R-1492, phosglycin, rotenone, schradan, sebufos, selamectin, sophamide, SSI-121, sulfiram, sulfluramid, sulfotep, sulfur, diflovidazin, tau-fluvalinate, TEPP, terbam, tetradifon, tetrasul, thiafenox, thiocarboxime, thiofanox, thiometon, thioquinox, thuringiensin, triamiphos, triarathene, triazophos, triazuron, trifenofos, trinactin, vamidothion, vaniliprole, bethoxazin, copper dioctanoate, copper sulfate, cybutryne, dichlone, dichlorophen, endothal, fentin, hydrated lime, nabam, quinoclamine, quinonamid, simazine, triphenyltin acetate, triphenyltin hydroxide, crufomate, piperazine, thiophanate, chloralose, fenthion, pyridin-4-amine, strychnine, 1-hydroxy-1H-pyridine-2-thione, 4-(quinoxalin-2-ylamino)benzenesulfonamide, 8-hydroxyquinoline sulfate, bronopol, copper hydroxide, cresol, dipyrithione, dodicin, fenaminosulf, formaldehyde, hydrargaphen, kasugamycin, kasugamycin hydrochloride hydrate, nickel bis(dimethyldithiocarbamate), nitrapyrin, octhilinone, oxolinic acid, oxytetracycline, potassium hydroxyquinoline sulfate, probenazole, streptomycin, streptomycin sesquisulfate, tecloftalam, thiomersal, Adoxophyes orana GV, Agrobacterium radiobacter, Amblyseius spp., Anagrapha falcifera NPV, Anagrus atomus, Aphelinus abdominalis, Aphidius colemani, Aphidoletes aphidimyza, Autographa californica NPV, Bacillus sphaericus Neide, Beauveria brongniartii, Chrysoperla carnea, Cryptolaemus montrouzieri, Cydia pomonella GV, Dacnusa sibirica, Diglyphus isaea, Encarsia formosa, Eretmocerus eremicus, Heterorhabditis bacteriophora and H. megidis, Hippodamia convergens, Leptomastix dactylopii, Macrolophus caliginosus, Mamestra brassicae NPV, Metaphycus helvolus, Metarhizium anisopliae var. acridum, Metarhizium anisopliae var. anisopliae, Neodiprion sertifer NPV and N. lecontei NPV, Orius spp., Paecilomyces fumosoroseus, Phytoseiulus persimilis, Steinernema bibionis, Steinernema carpocapsae, Steinernema feltiae, Steinernema glaseri, Steinernema riobrave, Steinernema riobravis, Steinernema scapterisci, Steinernema spp., Trichogramma spp., Typhlodromus occidentalis, Verticillium lecanii, apholate, bisazir, busulfan, dimatif, hemel, hempa, metepa, methiotepa, methyl apholate, morzid, penfluron, tepa, thiohempa, thiotepa, tretamine, uredepa, (E)-dec-5-en-1-yl acetate with (E)-dec-5-en-1-ol, (E)-tridec-4-en-1-yl acetate, (E)-6-methylhept-2-en-4-ol, (E,Z)-tetradeca-4,10-dien-1-yl acetate, (Z)-dodec-7-en-1-yl acetate, (Z)-hexadec-11-enal, (Z)-hexadec-11-en-1-yl acetate, (Z)-hexadec-13-en-11-yn-1-yl acetate, (Z)-icos-13-en-10-one, (Z)-tetradec-7-en-1-al, (Z)-tetradec-9-en-1-ol, (Z)-tetradec-9-en-1-yl acetate, (7E,9Z)-dodeca-7,9-dien-1-yl acetate, (9Z,11E)-tetradeca-9,11-dien-1-yl acetate, (9Z,12E)-tetradeca-9,12-dien-1-yl acetate, 14-methyloctadec-1-ene, 4-methylnonan-5-ol with 4-methylnonan-5-one, alpha-multistriatin, brevicomin, codlelure, codlemone, cuelure, disparlure, dodec-8-en-1-yl acetate, dodec-9-en-1-yl acetate, dodeca-8,10-dien-1-yl acetate, dominicalure, ethyl 4-methyloctanoate, eugenol, frontalin, grandlure, grandlure I, grandlure II, grandlure III, grandlure IV, hexalure, ipsdienol, ipsenol, japonilure, lineatin, litlure, looplure, medlure, megatomoic acid, methyl eugenol, muscalure, octadeca-2,13-dien-1-yl acetate, octadeca-3,13-dien-1-yl acetate, orfralure, oryctalure, ostramone, siglure, sordidin, sulcatol, tetradec-11-en-1-yl acetate, trimedlure, trimedlure A, trimedlure B1, trimedlure B2, trimedlure C, trunc-call, 2-(octylthio)ethanol, butopyronoxyl, butoxy(polypropylene glycol), dibutyl adipate, dibutyl phthalate, dibutyl succinate, diethyltoluamide, dimethyl carbate, dimethyl phthalate, ethyl hexanediol, hexamide, methoquin-butyl, methylneodecanamide, oxamate, picaridin, 1-dichloro-1-nitroethane, 1,1-dichloro-2,2-bis(4-ethylphenyl)ethane, 1,2-dichloropropane with 1,3-dichloropropene, 1-bromo-2-chloroethane, 2,2,2-trichloro-1-(3,4-dichlorophenyl)ethyl acetate, 2,2-dichlorovinyl 2-ethylsulfinylethyl methyl phosphate, 2-(1,3-dithiolan-2-yl)phenyl dimethylcarbamate, 2-(2-butoxyethoxy)ethyl thiocyanate, 2-(4,5-dimethyl-1,3-dioxolan-2-yl)phenyl methylcarbamate, 2-(4-chloro-3,5-xylyloxy)ethanol, 2-chlorovinyl diethyl phosphate, 2-imidazolidone, 2-isovalerylindan-1.3-dione, 2-methyl(prop-2-ynyl)aminophenyl methylcarbamate, 2-thiocyanatoethyl laurate, 3-bromo-1-chloroprop-1-ene, 3-methyl-1-phenylpyrazol-5-yl dimethylcarbamate, 4-methyl(prop-2-ynyl)amino-3,5-xylyl methylcarbamate, 5,5-dimethyl-3-oxocyclohex-1-enyl dimethylcarbamate, acethion, acrylonitrile, aldrin, allosamidin, allyxycarb, alpha-ecdysone, aluminium phosphide, aminocarb, anabasine, athidathion, azamethiphos, Bacillus thuringiensis delta endotoxins, barium hexafluorosilicate, barium polysulfide, barthrin, Bayer 22/190, Bayer 22408, beta-cyfluthrin, beta-cypermethrin, bioethanomethrin, biopermethrin, bis(2-chloroethyl) ether, borax, bromfenvinfos, bromo-DDT, bufencarb, butacarb, butathiofos, butonate, calcium arsenate, calcium cyanide, carbon disulfide, carbon tetrachloride, cartap hydrochloride, cevadine, chlorbicyclen, chlordane, chlordecone, chloroform, chloropicrin, chlorphoxim, chlorprazophos, cis-resmethrin, cismethrin, clocythrin, copper acetoarsenite, copper arsenate, copper oleate, coumithoate, cryolite, CS 708, cyanofenphos, cyanophos, cyclethrin, cythioate, d-tetramethrin, DAEP, dazomet, decarbofuran, diamidafos, dicapthon, dichlofenthion, dicresyl, dicyclanil, dieldrin, diethyl 5-methylpyrazol-3-yl phosphate, dilor, dimefluthrin, dimetan, dimethrin, dimethylvinphos, dimetilan, dinoprop, dinosam, dinoseb, diofenolan, dioxabenzofos, dithicrofos, DSP, ecdysterone, EI 1642, EMPC, EPBP, etaphos, ethiofencarb, ethyl formate, ethylene dibromide, ethylene dichloride, ethylene oxide, EXD, fenchlorphos, fenethacarb, fenitrothion, fenoxacrim, fenpirithrin, fensulfothion, fenthion-ethyl, flucofuron, fosmethilan, fospirate, fosthietan, furathiocarb, furethrin, guazatine, guazatine acetates, sodium tetrathiocarbonate, halfenprox, HCH, HEOD, heptachlor, heterophos, HHDN, hydrogen cyanide, hyquincarb, IPSP, isazofos, isobenzan, isodrin, isofenphos, isolane, isoprothiolane, isoxathion, juvenile hormone I, juvenile hormone II, juvenile hormone III, kelevan, kinoprene, lead arsenate, leptophos, lirimfos, lythidathion, m-cumenyl methylcarbamate, magnesium phosphide, mazidox, mecarphon, menazon, mercurous chloride, mesulfenfos, metam, metam-potassium, metam-sodium, methanesulfonyl fluoride, methocrotophos, methoprene, methothrin, methoxychlor, methyl isothiocyanate, methylchloroform, methylene chloride, metoxadiazone, mirex, naftalofos, naphthalene, NC-170, nicotine, nicotine sulfate, nithiazine, nornicotine, O-5-dichloro-4-iodophenyl O-ethyl ethylphosphonothioate, O,O-diethyl O-4-methyl-2-oxo-2H-chromen-7-yl phosphorothioate, O,O-diethyl O-6-methyl-2-propylpyrimidin-4-yl phosphorothioate, O,O,O′,O′-tetrapropyl dithiopyrophosphate, oleic acid, para-dichlorobenzene, parathion-methyl, pentachlorophenol, pentachlorophenyl laurate, PH 60-38, phenkapton, phosnichlor, phosphine, phoxim-methyl, pirimetaphos, polychlorodicyclopentadiene isomers, potassium arsenite, potassium thiocyanate, precocene I, precocene II, precocene III, primidophos, profluthrin, promecarb, prothiofos, pyrazophos, pyresmethrin, quassia, quinalphos-methyl, quinothion, rafoxanide, resmethrin, rotenone, kadethrin, ryania, ryanodine, sabadilla, schradan, sebufos, SI-0009, thiapronil, sodium arsenite, sodium cyanide, sodium fluoride, sodium hexafluorosilicate, sodium pentachlorophenoxide, sodium selenate, sodium thiocyanate, sulcofuron, sulcofuron-sodium, sulfuryl fluoride, sulprofos, tar oils, tazimcarb, TDE, tebupirimfos, temephos, terallethrin, tetrachloroethane, thicrofos, thiocyclam, thiocyclam hydrogen oxalate, thionazin, thiosultap, thiosultap-sodium, tralomethrin, transpermethrin, triazamate, trichlormetaphos-3, trichloronat, trimethacarb, tolprocarb, triclopyricarb, triprene, veratridine, veratrine, XMC, zetamethrin, zinc phosphide, zolaprofos, meperfluthrin, tetramethylfluthrin, bis(tributyltin) oxide, bromoacetamide, ferric phosphate, niclosamide-olamine, tributyltin oxide, pyrimorph, trifenmorph, 1,2-dibromo-3-chloropropane, 1,3-dichloropropene, 3,4-dichlorotetrahydrothiophene 1,1-dioxide, 3-(4-chlorophenyl)-5-methylrhodanine, 5-methyl-6-thioxo-1,3,5-thiadiazinan-3-ylacetic acid, 6-isopentenylaminopurine, anisiflupurin, benclothiaz, cytokinins, DCIP, furfural, isamidofos, kinetin, Myrothecium verrucaria composition, tetrachlorothiophene, xylenols, zeatin, potassium ethylxanthate, acibenzolar, acibenzolar-S-methyl, Reynoutria sachalinensis extract, alpha-chlorohydrin, antu, barium carbonate, bisthiosemi, brodifacoum, bromadiolone, bromethalin, chlorophacinone, cholecalciferol, coumachlor, coumafuryl, coumatetralyl, crimidine, difenacoum, difethialone, diphacinone, ergocalciferol, flocoumafen, fluoroacetamide, flupropadine, flupropadine hydrochloride, norbormide, phosacetim, phosphorus, pindone, pyrinuron, scilliroside, sodium fluoroacetate, thallium sulfate, warfarin, 2-(2-butoxyethoxy)ethyl piperonylate, 5-(1,3-benzodioxol-5-yl)-3-hexylcyclohex-2-enone, farnesol with nerolidol, verbutin, MGK 264, piperonyl butoxide, piprotal, propyl isomer, S421, sesamex, sesasmolin, sulfoxide, anthraquinone, copper naphthenate, copper oxychloride, dicyclopentadiene, thiram, zinc naphthenate, ziram, imanin, ribavirin, chloroinconazide, mercuric oxide, thiophanate-methyl, azaconazole, bitertanol, bromuconazole, cyproconazole, difenoconazole, diniconazole, epoxiconazole, fenbuconazole, fluquinconazole, flusilazole, flutriafol, furametpyr, hexaconazole, imazalil, imibenconazole, ipconazole, metconazole, myclobutanil, paclobutrazole, pefurazoate, penconazole, prothioconazole, pyrifenox, prochloraz, propiconazole, pyrisoxazole, simeconazole, tebuconazole, tetraconazole, triadimefon, triadimenol, triflumizole, triticonazole, ancymidol, fenarimol, nuarimol, bupirimate, dimethirimol, ethirimol, dodemorph, fenpropidin, fenpropimorph, spiroxamine, tridemorph, cyprodinil, mepanipyrim, pyrimethanil, fenpiclonil, fludioxonil, benalaxyl, furalaxyl, metalaxyl, R-metalaxyl, ofurace, oxadixyl, carbendazim, debacarb, fuberidazole, thiabendazole, chlozolinate, dichlozoline, myclozoline, procymidone, vinclozoline, boscalid, carboxin, fenfuram, flutolanil, mepronil, oxycarboxin, penthiopyrad, thifluzamide, dodine, iminoctadine, azoxystrobin, dimoxystrobin, enestroburin, fenaminstrobin, flufenoxystrobin, fluoxastrobin, kresoxim-methyl, metominostrobin, trifloxystrobin, orysastrobin, picoxystrobin, pyraclostrobin, pyrametostrobin, pyraoxystrobin, ferbam, mancozeb, maneb, metiram, propineb, zineb, captafol, captan, fluoroimide, folpet, tolylfluanid, bordeaux mixture, copper oxide, mancopper, oxine-copper, nitrothal-isopropyl, edifenphos, iprobenphos, phosdiphen, tolclofos-methyl, anilazine, benthiavalicarb, blasticidin-S, chloroneb, chlorothalonil, cyflufenamid, cymoxanil, cyclobutrifluram, diclocymet, diclomezine, dicloran, diethofencarb, dimethomorph, flumorph, dithianon, ethaboxam, etridiazole, famoxadone, fenamidone, fenoxanil, ferimzone, fluazinam, flumetylsulforim, fluopicolide, fluoxytioconazole, flusulfamide, fluxapyroxad, fenhexamid, fosetylaluminium, hymexazol, iprovalicarb, cyazofamid, methasulfocarb, metrafenone, pencycuron, phthalide, polyoxins, propamocarb, pyribencarb, proquinazid, pyroquilon, pyriofenone, quinoxyfen, quintozene, tiadinil, triazoxide, tricyclazole, triforine, validamycin, valifenalate, zoxamide, mandipropamid, flubeneteram, isopyrazam, sedaxane, benzovindiflupyr, pydiflumetofen, 3-difluoromethyl-1-methyl-1H-pyrazole-4-carboxylic acid (3′,4′,5′-trifluoro-biphenyl-2-yl)-amide, isoflucypram, isotianil, dipymetitrone, 6-ethyl-5,7-dioxo-pyrrolo[4,5][1,4]dithiino[1,2-c]isothiazole-3-carbonitrile, 2-(difluoromethyl)-N-[3-ethyl-1,1-dimethyl-indan-4-yl]pyridine-3-carboxamide, 4-(2,6-difluorophenyl)-6-methyl-5-phenyl-pyridazine-3-carbonitrile, (R)-3-(difluoromethyl)-1-methyl-N-[1,1,3-trimethylindan-4-yl]pyrazole-4-carboxamide, 4-(2-bromo-4-fluoro-phenyl)-N-(2-chloro-6-fluoro-phenyl)-2,5-dimethyl-pyrazol-3-amine, 4-(2-bromo- 4-fluorophenyl)-N-(2-chloro- 6-fluorophenyl)-1, 3-dimethyl-1H-pyrazol-5-amine, fluindapyr, coumethoxystrobin (jiaxiangjunzhi), Ivbenmixianan, dichlobentiazox, mandestrobin, 3-(4,4-difluoro-3,4-dihydro-3,3-dimethylisoquinolin-1-yl)quinolone, 2-[2-fluoro-6-[(8-fluoro-2-methyl-3-quinolyl)oxy]phenyl]propan-2-ol, oxathiapiprolin, tert-butyl N-[6-[[[(1-methyltetrazol-5-yl)-phenyl-methylene]amino]oxymethyl]-2-pyridyl]carbamate, pyraziflumid, inpyrfluxam, trolprocarb, mefentrifluconazole, ipfentrifluconazole, 2-(difluoromethyl)-N-[(3R)-3-ethyl-1,1-dimethyl-indan-4-yl]pyridine-3-carboxamide,N′-(2,5-dimethyl-4-phenoxy-phenyl)-N-ethyl-N-methyl-formamidine, N′-[4-(4,5-dichlorothiazol-2-yl)oxy-2,5-dimethyl-phenyl]-N-ethyl-N-methyl-formamidine, [2-[3-[2-[1-[2-[3,5-bis(difluoromethyl)pyrazol-1-yl]acetyl]-4-piperidyl]thiazol-4-yl]-4,5-dihydroisoxazol-5-yl]-3-chloro-phenyl]methanesulfonate, but-3-ynyl N-[6-[[(Z)-[(1-methyltetrazol-5-yl)-phenyl-methylene]amino]oxymethyl]-2-pyridyl]carbamate, methyl N-[[5-[4-(2,4-dimethylphenyl)triazol-2-yl]-2-methyl-phenyl]methyl]carbamate, 3-chloro-6-methyl-5-phenyl-4-(2,4,6-trifluorophenyl)pyridazine, pyridachlometyl, 3-(difluoromethyl)-1-methyl-N-[1,1,3-trimethylindan-4-yl]pyrazole-4-carboxamide, 1-[2-[[1-(4-chlorophenyl)pyrazol-3-yl]oxymethyl]-3-methyl-phenyl]-4-methyl-tetrazol-5-one, 1-methyl-4-[3-methyl-2-[[2-methyl-4-(3,4,5-trimethylpyrazol-1-yl)phenoxy]methyl]phenyl]tetrazol-5-one, aminopyrifen, ametoctradin, amisulbrom, penflufen, (Z,2E)-5-[1-(4-chlorophenyl)pyrazol-3-yl]oxy-2-methoxyimino-N,3-dimethyl-pent-3-enamide, florylpicoxamid, fenpicoxamid, metarylpicoxamid, tebufloquin, ipflufenoquin, quinofumelin, isofetamid, ethyl 1-[[4-[[2-(trifluoromethyl)-1,3-dioxolan-2-yl]methoxy]phenyl]methyl]pyrazole-3-carboxylate (may be prepared from the methods described in WO2020/056090), ethyl 1-[[4-[(Z)-2-ethoxy-3,3,3-trifluoro-prop-1-enoxy]phenyl]methyl]pyrazole-3-carboxylate (may be prepared from the methods described in WO2020/056090), methyl N-[[4-[1-(4-cyclopropyl-2,6-difluoro-phenyl)pyrazol-4-yl]-2-methyl-phenyl]methyl]carbamate (may be prepared from the methods described in WO2020/097012), methyl N-[[4-[1-(2,6-difluoro-4-isopropyl-phenyl)pyrazol-4-yl]-2-methyl-phenyl]methyl]carbamate (may be prepared from the methods described in WO2020/097012), 6-chloro-3-(3-cyclopropyl-2-fluoro-phenoxy)-N-[2-(2,4-dimethylphenyl)-2,2-difluoro-ethyl]-5-methyl-pyridazine-4-carboxamide (may be prepared from the methods described in WO2020/109391), 6-chloro-N-[2-(2-chloro-4-methyl-phenyl)-2,2-difluoro-ethyl]-3-(3-cyclopropyl-2-fluoro-phenoxy)-5-methyl-pyridazine-4-carboxamide (may be prepared from the methods described in WO2020/109391), 6-chloro-3-(3-cyclopropyl-2-fluoro-phenoxy)-N-[2-(3,4-dimethylphenyl)-2,2-difluoro-ethyl]-5-methyl-pyridazine-4-carboxamide (may be prepared from the methods described in WO2020/109391),N-[2-[2,4-dichloro-phenoxy]phenyl]-3-(difluoromethyl)-1-methyl-pyrazole-4-carboxamide, N-[2-[2-chloro-4-(trifluoromethyl)phenoxy]phenyl]-3-(difluoromethyl)-1-methyl-pyrazole-4-carboxamide, benzothiostrobin, phenamacril, 5-amino-1,3,4-thiadiazole-2-thiol zinc salt (2:1), fluopyram, flufenoxadiazam, flutianil, fluopimomide, pyrapropoyne, picarbutrazox, 2-(difluoromethyl)-N-(3-ethyl-1,1-dimethyl-indan-4-yl)pyridine-3-carboxamide, 2-(difluoromethyl)-N-((3R)-1,1,3-trimethylindan-4-yl) pyridine-3-carboxamide, 4-[[6-[2-(2,4-difluorophenyl)-1,1-difluoro-2-hydroxy-3-(1,2,4-triazol-1-yl)propyl]-3-pyridyl]oxy]benzonitrile, metyltetraprole, α-(1,1-dimethylethyl)-α-[4′-(trifluoromethoxy) [1,1′-biphenyl]-4-yl]-5-pyrimidinemethanol, fluoxapiprolin, enoxastrobin, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate (these compounds may be prepared from the methods described in WO2020/079111), methyl (Z)-2-(5-cyclohexyl-2-methyl-phenoxy)-3-methoxy-prop-2-enoate, methyl (z)-2-(5-cyclopentyl-2-methyl-phenoxy)-3-methoxy-prop-2-enoate (these compounds may be prepared from the methods described in WO2020/193387), 4-[[6-[2-(2,4-difluorophenyl)-1,1-difluoro-2-hydroxy-3-(1,2,4-triazol-1-yl)propyl]-3-pyridyl]oxy]benzonitrile, 4-[[6-[2-(2,4-difluorophenyl)-1,1-difluoro-2-hydroxy-3-(5-sulfanyl-1,2,4-triazol-1-yl)propyl]-3-pyridyl]oxy]benzonitrile, 4-[[6-[2-(2,4-difluorophenyl)-1,1-difluoro-2-hydroxy-3-(5-thioxo-4H-1,2,4-triazol-1-yl)propyl]-3-pyridyl]oxy]benzonitrile, trinexapac, coumoxystrobin, zhongshengmycin, thiodiazole copper, zinc thiazole, amectotractin, iprodione, seboctylamine, N′-[5-bromo-2-methyl-6-[(1S)-1-methyl-2-propoxy-ethoxy]-3-pyridyl]-N-ethyl-N-methyl-formamidine, N′-[5-bromo-2-methyl-6-[(1R)-1-methyl-2-propoxy-ethoxy]-3-pyridyl]-N-ethyl-N-methyl-formamidine, N′-[5-bromo-2-methyl-6-(1-methyl-2-propoxy-ethoxy)-3-pyridyl]-N-ethyl-N-methyl-formamidine, N′-[5-chloro-2-methyl-6-(1-methyl-2-propoxy-ethoxy)-3-pyridyl]-N-ethyl-N-methyl-formamidine, N′-[5-bromo-2-methyl-6-(1-methyl-2-propoxy-ethoxy)-3-pyridyl]-N-isopropyl-N-methyl-formamidine (these compounds may be prepared from the methods described in WO2015/155075); N′-[5-bromo-2-methyl-6-(2-propoxypropoxy)-3-pyridyl]-N-ethyl-N-methyl-formamidine (this compound may be prepared from the methods described in IPCOM000249876D); N-isopropyl-N′-[5-methoxy-2-methyl-4-(2,2,2-trifluoro-1-hydroxy-1-phenyl-ethyl)phenyl]-N-methyl-formamidine, N′-[4-(1-cyclopropyl-2,2,2-trifluoro-1-hydroxy-ethyl)-5-methoxy-2-methyl-phenyl]-N-isopropyl-N-methyl-formamidine (these compounds may be prepared from the methods described in WO2018/228896); N-ethyl-N′-[5-methoxy-2-methyl-4-[(2-trifluoromethyl)oxetan-2-yl]phenyl]-N-methyl-formamidine, N-ethyl-N′-[5-methoxy-2-methyl-4-[(2-trifuoromethyl)tetrahydrofuran-2-yl]phenyl]-N-methyl-formamidine (these compounds may be prepared from the methods described in WO2019/110427); N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide (these compounds may be prepared from the methods described in WO2017/153380); 1-(6,7-dimethylpyrazolo[1,5-a]pyridin-3-yl)-4,4,5-trifluoro-3,3-dimethyl-isoquinoline, 1-(6,7-dimethylpyrazolo[1,5-a]pyridin-3-yl)-4,4,6-trifluoro-3,3-dimethyl-isoquinoline, 4,4-difluoro-3,3-dimethyl-1-(6-methylpyrazolo[1,5-a]pyridin-3-yl)isoquinoline, 4,4-difluoro-3,3-dimethyl-1-(7-methylpyrazolo[1,5-a]pyridin-3-yl)isoquinoline, 1-(6-chloro-7-methyl-pyrazolo[1,5-a]pyridin-3-yl)-4,4-difluoro-3,3-dimethyl-isoquinoline (these compounds may be prepared from the methods described in WO2017/025510); 1-(4,5-dimethylbenzimidazol-1-yl)-4,4,5-trifluoro-3,3-dimethyl-isoquinoline, 1-(4,5-dimethylbenzimidazol-1-yl)-4,4-difluoro-3,3-dimethyl-isoquinoline, 6-chloro-4,4-difluoro-3,3-dimethyl-1-(4-methylbenzimidazol-1-yl)isoquinoline, 4,4-difluoro-1-(5-fluoro-4-methyl-benzimidazol-1-yl)-3,3-dimethyl-isoquinoline, 3-(4,4-difluoro-3,3-dimethyl-1-isoquinolyl)-7,8-dihydro-6H-cyclopenta[e]benzimidazole (these compounds may be prepared from the methods described in WO2016/156085); N-methoxy-N-[[4-[5-(trifluoromethyl)-1,2,4-oxadiazol-3-yl]phenyl]methyl]cyclopropane carboxamide, N,2-dimethoxy-N-[[4-[5-(trifluoromethyl)-1,2,4-oxadiazol-3-yl]phenyl]methyl]propanamide, N-ethyl-2-methyl-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, 3-ethyl-1-methoxy-1-[[4-[5-(trifluoromethyl)-1,2,4-oxadiazol-3-yl]phenyl]methyl]urea, N-[[4-[5-(trifluoromethyl)-1,2,4-oxadiazol-3-yl]phenyl]methyl]propanamide, 4,4-dimethyl-2-[[4-[5-(trifluoromethyl)-1,2,4-oxadiazol-3-yl]phenyl]methyl]isoxazolidin-3-one, 5,5-dimethyl-2-[[4-[5-(trifluoromethyl)-1,2,4-oxadiazol-3-yl]phenyl]methyl]isoxazolidin-3-one, ethyl 1-[[4-[5-(trifluoromethyl)-1,2,4-oxadiazol-3-yl]phenyl]methyl]pyrazole-4-carboxylate, N,N-dimethyl-1-[[4-[5-(trifluoromethyl)-1,2,4-oxadiazol-3-yl]phenyl]methyl]-1,2,4-triazol-3-amine (these compounds may be prepared from the methods described in WO2017/055473, WO2017/055469, WO2017/093348 and WO2017/118689); 2-[6-(4-chlorophenoxy)-2-(trifluoromethyl)-3-pyridyl]-1-(1,2,4-triazol-1-yl)propan-2-ol (this compound may be prepared from the methods described in WO2017/029179); 2-[6-(4-bromophenoxy)-2-(trifluoromethyl)-3-pyridyl]-1-(1,2,4-triazol-1-yl)propan-2-ol (this compound may be prepared from the methods described in WO2017/029179); 3-[2-(1-chlorocyclopropyl)-3-(2-fluorophenyl)-2-hydroxy-propyl]imidazole-4-carbonitrile (this compound may be prepared from the methods described in WO2016/156290); 3-[2-(1-chlorocyclopropyl)-3-(3-chloro-2-fluoro-phenyl)-2-hydroxy-propyl]imidazole-4-carbonitrile (this compound may be prepared from the methods described in WO2016/156290); (4-phenoxyphenyl)methyl 2-amino-6-methyl-pyridine-3-carboxylate (this compound may be prepared from the methods described in WO2014/006945); 2,6-Dimethyl-1H,5H-[1,4]dithiino[2,3-c:5,6-c′]dipyrrole-1,3,5,7(2H,6H)-tetrone (this compound may be prepared from the methods described in WO2011/138281) N-methyl-4-[5-(trifluoromethyl)-1,2,4-oxadiazol-3-yl]benzenecarbothioamide; N-methyl-4-[5-(trifluoromethyl)-1,2,4-oxadiazol-3-yl]benzamide; (Z,2E)-5-[1-(2,4-dichlorophenyl)pyrazol-3-yl]oxy-2-methoxyimino-N,3-dimethyl-pent-3-enamide (this compound may be prepared from the methods described in WO2018/153707); N′-(2-chloro-5-methyl-4-phenoxy-phenyl)-N-ethyl-N-methyl-formamidine; N′-[2-chloro-4-(2-fluorophenoxy)-5-methyl-phenyl]-N-ethyl-N-methyl-formamidine (this compound may be prepared from the methods described in WO2016/202742); 2-(difluoromethyl)-N-[(3S)-3-ethyl-1,1-dimethyl-indan-4-yl]pyridine-3-carboxamide (this compound may be prepared from the methods described in WO2014/095675); (5-methyl-2-pyridyl)-[4-[5-(trifluoromethyl)-1,2,4-oxadiazol-3-yl]phenyl]methanone, (3-methylisoxazol-5-yl)-[4-[5-(trifluoromethyl)-1,2,4-oxadiazol-3-yl]phenyl]methanone (these compounds may be prepared from the methods described in WO2017/220485); 2-oxo-N-propyl-2-[4-[5-(trifluoromethyl)-1,2,4-oxadiazol-3-yl]phenyl]acetamide (this compound may be prepared from the methods described in WO2018/065414); ethyl 1-[[5-[5-(trifluoromethyl)-1,2,4-oxadiazol-3-yl]-2-thienyl]methyl]pyrazole-4-carboxylate (this compound may be prepared from the methods described in WO2018/158365); 2,2-difluoro-N-methyl-2-[4-[5-(trifluoromethyl)-1,2,4-oxadiazol-3-yl]phenyl]acetamide, N-[(E)-methoxyiminomethyl]-4-[5-(trifluoromethyl)-1,2,4-oxadiazol-3-yl]benzamide, N—[(Z)-methoxyiminomethyl]-4-[5-(trifluoromethyl)-1,2,4-oxadiazol-3-yl]benzamide, N—[N-methoxy-methyl-carbonimidoyl]-4-[5-(trifluoromethyl)-1,2,4-oxadiazol-3-yl]benzamide (these compounds may be prepared from the methods described in WO2018/202428). The compounds of the invention may also be used in combination with anthelmintic agents. Such anthelmintic agents include, compounds selected from the macrocyclic lactone class of compounds such as ivermectin, avermectin, abamectin, emamectin, eprinomectin, doramectin, selamectin, moxidectin, nemadectin and milbemycin derivatives as described in EP0357460, EP0444964 and EP0594291. Additional anthelmintic agents include semisynthetic and biosynthetic avermectin/milbemycin derivatives such as those described in U.S. Pat. No. 5,015,630, WO9415944 and WO9522552. Additional anthelmintic agents include the benzimidazoles such as albendazole, cambendazole, fenbendazole, flubendazole, mebendazole, oxfendazole, oxibendazole, parbendazole, and other members of the class. Additional anthelmintic agents include imidazothiazoles and tetrahydropyrimidines such as tetramisole, levamisole, pyrantel pamoate, oxantel or morantel. Additional anthelmintic agents include flukicides, such as triclabendazole and clorsulon and the cestocides, such as praziquantel and epsiprantel.

The compounds of the invention may be used in combination with derivatives and analogues of the paraherquamide/marcfortine class of anthelmintic agents, as well as the antiparasitic oxazolines such as those disclosed in U.S. Pat. Nos. 5,478,855, 4,639,771 and DE-19520936.

The compounds of the invention may be used in combination with derivatives and analogues of the general class of dioxomorpholine antiparasitic agents as described in WO9615121 and also with anthelmintic active cyclic depsipeptides such as those described in WO9611945, WO9319053, WO9325543, EP0626375, EP0382173, WO9419334, EP0382173, and EP0503538.

The compounds of the invention may be used in combination with other ectoparasiticides; for example, fipronil; pyrethroids; organophosphates; insect growth regulators such as lufenuron; ecdysone agonists such as tebufenozide and the like; neonicotinoids such as imidacloprid and the like.

The compounds of the invention may be used in combination with terpene alkaloids, for example those described in WO95/19363 or WO04/72086, particularly the compounds disclosed therein.

Other examples of such biologically active compounds that the compounds of the invention may be used in combination with include but are not restricted to the following:

Organophosphates: acephate, azamethiphos, azinphos-ethyl, azinphos-methyl, bromophos, bromophos-ethyl, cadusafos, chlorethoxyphos, chlorpyrifos, chlorfenvinphos, chlormephos, demeton, demeton-S-methyl, demeton-S-methyl sulphone, dialifos, diazinon, dichlorvos, dicrotophos, dimethoate, disulfoton, ethion, ethoprophos, etrimfos, famphur, fenamiphos, fenitrothion, fensulfothion, fenthion, flupyrazofos, fonofos, formothion, fosthiazate, heptenophos, isazophos, isothioate, isoxathion, malathion, methacriphos, methamidophos, methidathion, methyl-parathion, mevinphos, monocrotophos, naled, omethoate, oxydemeton-methyl, paraoxon, parathion, parathion-methyl, phenthoate, phosalone, phosfolan, phosphocarb, phosmet, phosphamidon, phorate, phoxim, pirimiphos, pirimiphos-methyl, profenofos, propaphos, proetamphos, prothiofos, pyraclofos, pyridapenthion, quinalphos, sulprophos, temephos, terbufos, tebupirimfos, tetrachlorvinphos, thimeton, triazophos, trichlorfon, vamidothion.

Carbamates: alanycarb, aldicarb, 2-sec-butylphenyl methylcarbamate, benfuracarb, carbaryl, carbofuran, carbosulfan, cloethocarb, ethiofencarb, fenoxycarb, fenthiocarb, furathiocarb, HCN-801, isoprocarb, indoxacarb, methiocarb, methomyl, 5-methyl-m-cumenylbutyryl(methyl)carbamate, oxamyl, pirimicarb, propoxur, thiodicarb, thiofanox, triazamate, UC-51717.

Pyrethroids: acrinathin, allethrin, alphametrin, 5-benzyl-3-furylmethyl (E)-(1R)-cis-2,2-dimethyl-3-(2-oxothiolan-3-ylidenemethyl)cyclopropanecarboxylate, bifenthrin, beta-cyfluthrin, cyfluthrin, α-cypermethrin, beta-cypermethrin, bioallethrin, bioallethrin((S)-cyclopentylisomer), bioresmethrin, bifenthrin, NCI-85193, cycloprothrin, cyhalothrin, cythithrin, cyphenothrin, deltamethrin, empenthrin, esfenvalerate, ethofenprox, fenfluthrin, fenpropathrin, fenvalerate, flucythrinate, flumethrin, fluvalinate (D isomer), imiprothrin, cyhalothrin, lambda-cyhalothrin, permethrin, phenothrin, prallethrin, pyrethrins (natural products), resmethrin, tetramethrin, transfluthrin, theta-cypermethrin, silafluofen, t-fluvalinate, tefluthrin, tralomethrin, Zeta-cypermethrin.

Arthropod growth regulators: a) chitin synthesis inhibitors: benzoylureas: chlorfluazuron, diflubenzuron, fluazuron, flucycloxuron, flufenoxuron, hexaflumuron, lufenuron, novaluron, teflubenzuron, triflumuron, buprofezin, diofenolan, hexythiazox, etoxazole, chlorfentazine; b) ecdysone antagonists: halofenozide, methoxyfenozide, tebufenozide; c) juvenoids: pyriproxyfen, methoprene (including S-methoprene), fenoxycarb; d) lipid biosynthesis inhibitors: spirodiclofen.

Other antiparasitics: acequinocyl, amitraz, AKD-1022, ANS-118, azadirachtin, Bacillus thuringiensis, bensultap, bifenazate, binapacryl, bromopropylate, BTG-504, BTG-505, camphechlor, cartap, chlorobenzilate, chlordimeform, chlorfenapyr, chromafenozide, clothianidine, cyromazine, diacloden, diafenthiuron, DBI-3204, dinactin, dihydroxymethyldihydroxypyrrolidine, dinobuton, dinocap, endosulfan, ethiprole, ethofenprox, fenazaquin, flumite, MTI-800, fenpyroximate, fluacrypyrim, flubenzimine, flubrocythrinate, flufenzine, flufenprox, fluproxyfen, halofenprox, hydramethylnon, IKI-220, kanemite, NC-196, neem guard, nidinorterfuran, nitenpyram, SD-35651, WL-108477, pirydaryl, propargite, protrifenbute, pymethrozine, pyridaben, pyrimidifen, NC-1111, R-195, RH-0345, RH-2485, RYI-210, S-1283, S-1833, SI-8601, silafluofen, silomadine, spinosad, tebufenpyrad, tetradifon, tetranactin, thiacloprid, thiocyclam, thiamethoxam, tolfenpyrad, triazamate, triethoxyspinosyn, trinactin, verbutin, vertalec, YI-5301.

Biological agents: Bacillus thuringiensis ssp. aizawai, kurstaki, Bacillus thuringiensis delta endotoxin, baculovirus, entomopathogenic bacteria, virus and fungi.

Bactericides: chlortetracycline, oxytetracycline, streptomycin.

Other biological agents: enrofloxacin, febantel, penethamate, moloxicam, cefalexin, kanamycin, pimobendan, clenbuterol, omeprazole, tiamulin, benazepril, pyriprole, cefquinome, florfenicol, buserelin, cefovecin, tulathromycin, ceftiour, carprofen, metaflumizone, praziquarantel, triclabendazole.

The following mixtures of the compounds of Formula (I) with active ingredients are preferred. The abbreviation “TX” means one compound selected from compounds of formula (I), (I-A), (I-A1), (I-A2), (I-A3), (I-A4), (I-A5), (I-A6), or (I-A7), or compounds selected from compounds listed in Tables C-1 to C-36, or compounds listed in Table P (below), and a compound selected from the group of substances consisting of petroleum oils+TX, 1,1-bis(4-chlorophenyl)-2-ethoxyethanol+TX, 2,4-dichlorophenyl benzenesulfonate+TX, 2-fluoro-N-methyl-N-1-naphthylacetamide+TX, 4-chlorophenyl phenyl sulfone+TX, acetoprole+TX, aldoxycarb+TX, amidithion+TX, amidothioate+TX, amiton+TX, amiton hydrogen oxalate+TX, amitraz+TX, aramite+TX, arsenous oxide+TX, azobenzene+TX, azothoate+TX, benomyl+TX, benoxafos+TX, benzyl benzoate+TX, bixafen+TX, brofenvalerate+TX, bromocyclen+TX, bromophos+TX, bromopropylate+TX, buprofezin+TX, butocarboxim+TX, butoxycarboxim+TX, butylpyridaben+TX, calcium polysulfide+TX, camphechlor+TX, carbanolate+TX, carbophenothion+TX, cymiazole+TX, chinomethionat+TX, chlorbenside+TX, chlordimeform+TX, chlordimeform hydrochloride+TX, chlorfenethol+TX, chlorfenson+TX, chlorfensulfide+TX, chlorobenzilate+TX, chloromebuform+TX, chloromethiuron+TX, chloropropylate+TX, chlorthiophos+TX, cinerin I+TX, cinerin II+TX, cinerins+TX, closantel+TX, coumaphos+TX, crotamiton+TX, crotoxyphos+TX, cufraneb+TX, cyanthoate+TX, DCPM+TX, DDT+TX, demephion+TX, demephion-O+TX, demephion-S+TX, demeton-methyl+TX, demeton-O+TX, demeton-O-methyl+TX, demeton-S+TX, demeton-S-methyl+TX, demeton-S-methylsulfon+TX, dichlofluanid+TX, dichlorvos+TX, dicliphos+TX, dienochlor+TX, dimefox+TX, dinex+TX, dinex-diclexine+TX, dinocap-4+TX, dinocap-6+TX, dinocton+TX, dinopenton+TX, dinosulfon+TX, dinoterbon+TX, dioxathion+TX, diphenyl sulfone+TX, disulfiram+TX, DNOC+TX, dofenapyn+TX, doramectin+TX, endothion+TX, eprinomectin+TX, ethoate-methyl+TX, etrimfos+TX, fenazaflor+TX, fenbutatin oxide+TX, fenothiocarb+TX, fenpyrad+TX, fenpyroximate+TX, fenpyrazamine+TX, fenson+TX, fentrifanil+TX, flubenzimine+TX, flucycloxuron+TX, fluenetil+TX, fluorbenside+TX, FMC 1137+TX, formetanate+TX, formetanate hydrochloride+TX, formparanate+TX, gamma-HCH+TX, glyodin+TX, halfenprox+TX, hexadecyl cyclopropanecarboxylate+TX, isocarbophos+TX, jasmolin I+TX, jasmolin II+TX, jodfenphos+TX, lindane+TX, malonoben+TX, mecarbam+TX, mephosfolan+TX, mesulfen+TX, methacrifos+TX, methyl bromide+TX, metolcarb+TX, mexacarbate+TX, milbemycin oxime+TX, mipafox+TX, monocrotophos+TX, morphothion+TX, moxidectin+TX, naled+TX, 4-chloro-2-(2-chloro-2-methyl-propyl)-5-[(6-iodo-3-pyridyl)methoxy]pyridazin-3-one+TX, nifluridide+TX, nikkomycins+TX, nitrilacarb+TX, nitrilacarb 1:1 zinc chloride complex+TX, omethoate+TX, oxydeprofos+TX, oxydisulfoton+TX, pp′-DDT+TX, parathion+TX, permethrin+TX, phenkapton+TX, phosalone+TX, phosfolan+TX, phosphamidon+TX, polychloroterpenes+TX, polynactins+TX, proclonol+TX, promacyl+TX, propoxur+TX, prothidathion+TX, prothoate+TX, pyrethrin I+TX, pyrethrin II+TX, pyrethrins+TX, pyridaphenthion+TX, pyrimitate+TX, quinalphos+TX, quintiofos+TX, R-1492+TX, phosglycin+TX, rotenone+TX, schradan+TX, sebufos+TX, selamectin+TX, sophamide+TX, SSI-121+TX, sulfiram+TX, sulfluramid+TX, sulfotep+TX, sulfur+TX, diflovidazin+TX, tau-fluvalinate+TX, TEPP+TX, terbam+TX, tetradifon+TX, tetrasul+TX, thiafenox+TX, thiocarboxime+TX, thiofanox+TX, thiometon+TX, thioquinox+TX, thuringiensin+TX, triamiphos+TX, triarathene+TX, triazophos+TX, triazuron+TX, trifenofos+TX, trinactin+TX, vamidothion+TX, vaniliprole+TX, bethoxazin+TX, copper dioctanoate+TX, copper sulfate+TX, cybutryne+TX, dichlone+TX, dichlorophen+TX, endothal+TX, fentin+TX, hydrated lime+TX, nabam+TX, quinoclamine+TX, quinonamid+TX, simazine+TX, triphenyltin acetate+TX, triphenyltin hydroxide+TX, crufomate+TX, piperazine+TX, thiophanate+TX, chloralose+TX, fenthion+TX, pyridin-4-amine+TX, strychnine+TX, 1-hydroxy-1H-pyridine-2-thione+TX, 4-(quinoxalin-2-ylamino)benzenesulfonamide+TX, 8-hydroxyquinoline sulfate+TX, bronopol+TX, copper hydroxide+TX, cresol+TX, dipyrithione+TX, dodicin+TX, fenaminosulf+TX, formaldehyde+TX, hydrargaphen+TX, kasugamycin+TX, kasugamycin hydrochloride hydrate+TX, nickel bis(dimethyldithiocarbamate)+TX, nitrapyrin+TX, octhilinone+TX, oxolinic acid+TX, oxytetracycline+TX, potassium hydroxyquinoline sulfate+TX, probenazole+TX, streptomycin+TX, streptomycin sesquisulfate+TX, tecloftalam+TX, thiomersal+TX, Adoxophyes orana GV+TX, Agrobacterium radiobacter+TX, Amblyseius spp.+TX, Anagrapha falcifera NPV+TX, Anagrus atomus+TX, Aphelinus abdominalis+TX, Aphidius colemani+TX, Aphidoletes aphidimyza+TX, Autographa californica NPV+TX, Bacillus sphaericus Neide+TX, Beauveria brongniartii+TX, Chrysoperla carnea+TX, Cryptolaemus montrouzieri+TX, Cydia pomonella GV+TX, Dacnusa sibirica+TX, Diglyphus isaea+TX, Encarsia formosa+TX, Eretmocerus eremicus+TX, Heterorhabditis bacteriophora and H. megidis+TX, Hippodamia convergens+TX, Leptomastix dactylopii+TX, Macrolophus caliginosus+TX, Mamestra brassicae NPV+TX, Metaphycus helvolus+TX, Metarhizium anisopliae var. acridum+TX, Metarhizium anisopliae var. anisopliae+TX, Neodiprion sertifer NPV and N. lecontei NPV+TX, Orius spp.+TX, Paecilomyces fumosoroseus+TX, Phytoseiulus persimilis+TX, Steinernema bibionis+TX, Steinernema carpocapsae+TX, Steinernema feltiae+TX, Steinernema glaseri+TX, Steinernema riobrave+TX, Steinernema riobravis+TX, Steinernema scapterisci+TX, Steinernema spp.+TX, Trichogramma spp.+TX, Typhlodromus occidentalis+TX, Verticillium lecanii+TX, apholate+TX, bisazir+TX, busulfan+TX, dimatif+TX, hemel+TX, hempa+TX, metepa+TX, methiotepa+TX, methyl apholate+TX, morzid+TX, penfluron+TX, tepa+TX, thiohempa+TX, thiotepa+TX, tretamine+TX, uredepa+TX, (E)-dec-5-en-1-yl acetate with (E)-dec-5-en-1-ol+TX, (E)-tridec-4-en-1-yl acetate+TX, (E)-6-methylhept-2-en-4-ol+TX, (E,Z)-tetradeca-4,10-dien-1-yl acetate+TX, (Z)-dodec-7-en-1-yl acetate+TX, (Z)-hexadec-11-enal+TX, (Z)-hexadec-11-en-1-yl acetate+TX, (Z)-hexadec-13-en-11-yn-1-yl acetate+TX, (Z)-icos-13-en-10-one+TX, (Z)-tetradec-7-en-1-al+TX, (Z)-tetradec-9-en-1-ol+TX, (Z)-tetradec-9-en-1-yl acetate+TX, (7E,9Z)-dodeca-7,9-dien-1-yl acetate+TX, (9Z,11E)-tetradeca-9,11-dien-1-yl acetate+TX, (9Z,12E)-tetradeca-9,12-dien-1-yl acetate+TX, 14-methyloctadec-1-ene+TX, 4-methylnonan-5-ol with 4-methylnonan-5-one+TX, alpha-multistriatin+TX, brevicomin+TX, codlelure+TX, codlemone+TX, cuelure+TX, disparlure+TX, dodec-8-en-1-yl acetate+TX, dodec-9-en-1-yl acetate+TX, dodeca-8,10-dien-1-yl acetate+TX, dominicalure+TX, ethyl 4-methyloctanoate+TX, eugenol+TX, frontalin+TX, grandlure+TX, grandlure I+TX, grandlure II+TX, grandlure III+TX, grandlure IV+TX, hexalure+TX, ipsdienol+TX, ipsenol+TX, japonilure+TX, lineatin+TX, litlure+TX, looplure+TX, medlure+TX, megatomoic acid+TX, methyl eugenol+TX, muscalure+TX, octadeca-2,13-dien-1-yl acetate+TX, octadeca-3,13-dien-1-yl acetate+TX, orfralure+TX, oryctalure+TX, ostramone+TX, siglure+TX, sordidin+TX, sulcatol+TX, tetradec-11-en-1-yl acetate+TX, trimedlure+TX, trimedlure A+TX, trimedlure Bi+TX, trimedlure B₂+TX, trimedlure C+TX, trunc-call+TX, 2-(octylthio)-ethanol+TX, butopyronoxyl+TX, butoxy(polypropylene glycol)+TX, dibutyl adipate+TX, dibutyl phthalate+TX, dibutyl succinate+TX, diethyltoluamide+TX, dimethyl carbate+TX, dimethyl phthalate+TX, ethyl hexanediol+TX, hexamide+TX, methoquin-butyl+TX, methylneodecanamide+TX, oxamate+TX, picaridin+TX, 1-dichloro-1-nitroethane+TX, 1,1-dichloro-2,2-bis(4-ethylphenyl)ethane+TX, 1,2-dichloropropane with 1,3-dichloropropene+TX, 1-bromo-2-chloroethane+TX, 2,2,2-trichloro-1-(3,4-dichlorophenyl)ethyl acetate+TX, 2,2-dichlorovinyl 2-ethylsulfinylethyl methyl phosphate+TX, 2-(1,3-dithiolan-2-yl)phenyl dimethylcarbamate+TX, 2-(2-butoxyethoxy)ethyl thiocyanate+TX, 2-(4,5-dimethyl-1,3-dioxolan-2-yl)phenyl methylcarbamate+TX, 2-(4-chloro-3,5-xylyloxy)ethanol+TX, 2-chlorovinyl diethyl phosphate+TX, 2-imidazolidone+TX, 2-isovalerylindan-1,3-dione+TX, 2-methyl(prop-2-ynyl)aminophenyl methylcarbamate+TX, 2-thiocyanatoethyl laurate+TX, 3-bromo-1-chloroprop-1-ene+TX, 3-methyl-1-phenylpyrazol-5-yl dimethylcarbamate+TX, 4-methyl(prop-2-ynyl)amino-3,5-xylyl methylcarbamate+TX, 5,5-dimethyl-3-oxocyclohex-1-enyl dimethylcarbamate+TX, acethion+TX, acrylonitrile+TX, aldrin+TX, allosamidin+TX, allyxycarb+TX, alpha-ecdysone+TX, aluminium phosphide+TX, aminocarb+TX, anabasine+TX, athidathion+TX, azamethiphos+TX, Bacillus thuringiensis delta endotoxins+TX, barium hexafluorosilicate+TX, barium polysulfide+TX, barthrin+TX, Bayer 22/190+TX, Bayer 22408+TX, beta-cyfluthrin+TX, beta-cypermethrin+TX, bioethanomethrin+TX, biopermethrin+TX, bis(2-chloroethyl) ether+TX, borax+TX, bromfenvinfos+TX, bromo-DDT+TX, bufencarb+TX, butacarb+TX, butathiofos+TX, butonate+TX, calcium arsenate+TX, calcium cyanide+TX, carbon disulfide+TX, carbon tetrachloride+TX, cartap hydrochloride+TX, cevadine+TX, chlorbicyclen+TX, chlordane+TX, chlordecone+TX, chloroform+TX, chloropicrin+TX, chlorphoxim+TX, chlorprazophos+TX, cis-resmethrin+TX, cismethrin+TX, clocythrin+TX, copper acetoarsenite+TX, copper arsenate+TX, copper oleate+TX, coumithoate+TX, cryolite+TX, CS 708+TX, cyanofenphos+TX, cyanophos+TX, cyclethrin+TX, cythioate+TX, d-tetramethrin+TX, DAEP+TX, dazomet+TX, decarbofuran+TX, diamidafos+TX, dicapthon+TX, dichlofenthion+TX, dicresyl+TX, dicyclanil+TX, dieldrin+TX, diethyl 5-methylpyrazol-3-yl phosphate+TX, dilor+TX, dimefluthrin+TX, dimetan+TX, dimethrin+TX, dimethylvinphos+TX, dimetilan+TX, dinoprop+TX, dinosam+TX, dinoseb+TX, diofenolan+TX, dioxabenzofos+TX, dithicrofos+TX, DSP+TX, ecdysterone+TX, EI 1642+TX, EMPC+TX, EPBP+TX, etaphos+TX, ethiofencarb+TX, ethyl formate+TX, ethylene dibromide+TX, ethylene dichloride+TX, ethylene oxide+TX, EXD+TX, fenchlorphos+TX, fenethacarb+TX, fenitrothion+TX, fenoxacrim+TX, fenpirithrin+TX, fensulfothion+TX, fenthion-ethyl+TX, flucofuron+TX, fosmethilan+TX, fospirate+TX, fosthietan+TX, furathiocarb+TX, furethrin+TX, guazatine+TX, guazatine acetates+TX, sodium tetrathiocarbonate+TX, halfenprox+TX, HCH+TX, HEOD+TX, heptachlor+TX, heterophos+TX, HHDN+TX, hydrogen cyanide+TX, hyquincarb+TX, IPSP+TX, isazofos+TX, isobenzan+TX, isodrin+TX, isofenphos+TX, isolane+TX, isoprothiolane+TX, isoxathion+TX, juvenile hormone I+TX, juvenile hormone II+TX, juvenile hormone Ill+TX, kelevan+TX, kinoprene+TX, lead arsenate+TX, leptophos+TX, lirimfos+TX, lythidathion+TX, m-cumenyl methylcarbamate+TX, magnesium phosphide+TX, mazidox+TX, mecarphon+TX, menazon+TX, mercurous chloride+TX, mesulfenfos+TX, metam+TX, metam-potassium+TX, metam-sodium+TX, methanesulfonyl fluoride+TX, methocrotophos+TX, methoprene+TX, methothrin+TX, methoxychlor+TX, methyl isothiocyanate+TX, methylchloroform+TX, methylene chloride+TX, metoxadiazone+TX, mirex+TX, naftalofos+TX, naphthalene+TX, NC-170+TX, nicotine+TX, nicotine sulfate+TX, nithiazine+TX, nornicotine+TX, O-5-dichloro-4-iodophenyl O-ethyl ethylphosphonothioate+TX, O,O-diethyl O-4-methyl-2-oxo-2H-chromen-7-yl phosphorothioate+TX, O,O-diethyl O-6-methyl-2-propylpyrimidin-4-yl phosphorothioate+TX, O,O,O′,O′-tetrapropyl dithiopyrophosphate+TX, oleic acid+TX, para-dichlorobenzene+TX, parathion-methyl+TX, pentachlorophenol+TX, pentachlorophenyl laurate+TX, PH 60-38+TX, phenkapton+TX, phosnichlor+TX, phosphine+TX, phoxim-methyl+TX, pirimetaphos+TX, polychlorodicyclopentadiene isomers+TX, potassium arsenite+TX, potassium thiocyanate+TX, precocene I+TX, precocene II+TX, precocene Ill+TX, primidophos+TX, profluthrin+TX, promecarb+TX, prothiofos+TX, pyrazophos+TX, pyresmethrin+TX, quassia+TX, quinalphos-methyl+TX, quinothion+TX, rafoxanide+TX, resmethrin+TX, rotenone+TX, kadethrin+TX, ryania+TX, ryanodine+TX, sabadilla)+TX, schradan+TX, sebufos+TX, SI-0009+TX, thiapronil+TX, sodium arsenite+TX, sodium cyanide+TX, sodium fluoride+TX, sodium hexafluorosilicate+TX, sodium pentachlorophenoxide+TX, sodium selenate+TX, sodium thiocyanate+TX, sulcofuron+TX, sulcofuron-sodium+TX, sulfuryl fluoride+TX, sulprofos+TX, tar oils+TX, tazimcarb+TX, TDE+TX, tebupirimfos+TX, temephos+TX, terallethrin+TX, tetrachloroethane+TX, thicrofos+TX, thiocyclam+TX, thiocyclam hydrogen oxalate+TX, thionazin+TX, thiosultap+TX, thiosultap-sodium+TX, tralomethrin+TX, transpermethrin+TX, triazamate+TX, trichlormetaphos-3+TX, trichloronat+TX, trimethacarb+TX, tolprocarb+TX, triclopyricarb+TX, triprene+TX, veratridine+TX, veratrine+TX, XMC+TX, zetamethrin+TX, zinc phosphide+TX, zolaprofos+TX, and meperfluthrin+TX, tetramethylfluthrin+TX, bis(tributyltin) oxide+TX, bromoacetamide+TX, ferric phosphate+TX, niclosamide-olamine+TX, tributyltin oxide+TX, pyrimorph+TX, trifenmorph+TX, 1,2-dibromo-3-chloropropane+TX, 1,3-dichloropropene+TX, 3,4-dichlorotetrahydrothiophene 1,1-dioxide+TX, 3-(4-chlorophenyl)-5-methylrhodanine+TX, 5-methyl-6-thioxo-1,3,5-thiadiazinan-3-ylacetic acid+TX, 6-isopentenylaminopurine+TX, 2-fluoro-N-(3-methoxyphenyl)-9H-purin-6-amine+TX, benclothiaz+TX, cytokinins+TX, DCIP+TX, furfural+TX, isamidofos+TX, kinetin+TX, Myrothecium verrucaria composition+TX, tetrachlorothiophene+TX, xylenols+TX, zeatin+TX, potassium ethylxanthate+TX, acibenzolar+TX, acibenzolar-S-methyl+TX, Reynoutria sachalinensis extract+TX, alpha-chlorohydrin+TX, antu+TX, barium carbonate+TX, bisthiosemi+TX, brodifacoum+TX, bromadiolone+TX, bromethalin+TX, chlorophacinone+TX, cholecalciferol+TX, coumachlor+TX, coumafuryl+TX, coumatetralyl+TX, crimidine+TX, difenacoum+TX, difethialone+TX, diphacinone+TX, ergocalciferol+TX, flocoumafen+TX, fluoroacetamide+TX, flupropadine+TX, flupropadine hydrochloride+TX, norbormide+TX, phosacetim+TX, phosphorus+TX, pindone+TX, pyrinuron+TX, scilliroside+TX, sodium fluoroacetate+TX, thallium sulfate+TX, warfarin+TX, 2-(2-butoxyethoxy)ethyl piperonylate+TX, 5-(1,3-benzodioxol-5-yl)-3-hexylcyclohex-2-enone+TX, farnesol with nerolidol+TX, verbutin+TX, MGK 264+TX, piperonyl butoxide+TX, piprotal+TX, propyl isomer+TX, S421+TX, sesamex+TX, sesasmolin+TX, sulfoxide+TX, anthraquinone+TX, copper naphthenate+TX, copper oxychloride+TX, dicyclopentadiene+TX, thiram+TX, zinc naphthenate+TX, ziram+TX, imanin+TX, ribavirin+TX, mercuric oxide+TX, thiophanate-methyl+TX, azaconazole+TX, bitertanol+TX, bromuconazole+TX, cyproconazole+TX, difenoconazole+TX, diniconazole+TX, epoxiconazole+TX, fenbuconazole+TX, fluquinconazole+TX, flusilazole+TX, flutriafol+TX, furametpyr+TX, hexaconazole+TX, imazalil+TX, imibenconazole+TX, ipconazole+TX, metconazole+TX, myclobutanil+TX, paclobutrazole+TX, pefurazoate+TX, penconazole+TX, prothioconazole+TX, pyrifenox+TX, prochloraz+TX, propiconazole+TX, pyrisoxazole+TX, -simeconazole+TX, tebuconazole+TX, tetraconazole+TX, triadimefon+TX, triadimenol+TX, triflumizole+TX, triticonazole+TX, ancymidol+TX, fenarimol+TX, nuarimol+TX, bupirimate+TX, dimethirimol+TX, ethirimol+TX, dodemorph+TX, fenpropidin+TX, fenpropimorph+TX, spiroxamine+TX, tridemorph+TX, cyprodinil+TX, mepanipyrim+TX, pyrimethanil+TX, fenpiclonil+TX, fludioxonil+TX, benalaxyl+TX, furalaxyl+TX, metalaxyl-+TX, Rmetalaxyl+TX, ofurace+TX, oxadixyl+TX, carbendazim+TX, debacarb+TX, fuberidazole+TX, thiabendazole+TX, chlozolinate+TX, dichlozoline+TX, myclozoline+TX, procymidone+TX, vinclozoline+TX, boscalid+TX, carboxin+TX, fenfuram+TX, flutolanil+TX, mepronil+TX, oxycarboxin+TX, penthiopyrad+TX, thifluzamide+TX, dodine+TX, iminoctadine+TX, azoxystrobin+TX, dimoxystrobin+TX, enestroburin+TX, fenaminstrobin+TX, flufenoxystrobin+TX, fluoxastrobin+TX, kresoxim-methyl+TX, metominostrobin+TX, trifloxystrobin+TX, orysastrobin+TX, picoxystrobin+TX, pyraclostrobin+TX, pyrametostrobin+TX, pyraoxystrobin+TX, ferbam+TX, mancozeb+TX, maneb+TX, metiram+TX, propineb+TX, zineb+TX, captafol+TX, captan+TX, fluoroimide+TX, folpet+TX, tolylfluanid+TX, bordeaux mixture+TX, copper oxide+TX, mancopper+TX, oxine-copper+TX, nitrothal-isopropyl+TX, edifenphos+TX, iprobenphos+TX, phosdiphen+TX, tolclofos-methyl+TX, anilazine+TX, benthiavalicarb+TX, blasticidin-S+TX, chloroneb+TX, chlorothalonil+TX, cyflufenamid+TX, cymoxanil+TX, cyclobutrifluram+TX, diclocymet+TX, diclomezine+TX, dicloran+TX, diethofencarb+TX, dimethomorph+TX, flumorph+TX, dithianon+TX, ethaboxam+TX, etridiazole+TX, famoxadone+TX, fenamidone+TX, fenoxanil+TX, ferimzone+TX, fluazinam+TX, fluopicolide+TX, flusulfamide+TX, fluxapyroxad+TX, fenhexamid+TX, fosetyl-aluminium+TX, hymexazol+TX, iprovalicarb+TX, cyazofamid+TX, methasulfocarb+TX, metrafenone+TX, pencycuron+TX, phthalide+TX, polyoxins+TX, propamocarb+TX, pyribencarb+TX, proquinazid+TX, pyroquilon+TX, pyriofenone+TX, quinoxyfen+TX, quintozene+TX, tiadinil+TX, triazoxide+TX, tricyclazole+TX, triforine+TX, validamycin+TX, valifenalate+TX, zoxamide+TX, mandipropamid+TX, flubeneteram+TX, isopyrazam+TX, sedaxane+TX, benzovindiflupyr+TX, pydiflumetofen+TX, 3-difluoromethyl-1-methyl-1H-pyrazole-4-carboxylic acid (3′,4′,5′-trifluoro-biphenyl-2-yl)-amide+TX, isoflucypram+TX, isotianil+TX, dipymetitrone+TX, 6-ethyl-5,7-dioxo-pyrrolo[4,5][1,4]dithiino[1,2-c]isothiazole-3-carbonitrile+TX, 2-(difluoromethyl)-N-[3-ethyl-1,1-dimethyl-indan-4-yl]pyridine-3-carboxamide+TX, 4-(2,6-difluorophenyl)-6-methyl-5-phenyl-pyridazine-3-carbonitrile+TX, (R)-3-(difluoromethyl)-1-methyl-N-[1,1,3-trimethylindan-4-yl]pyrazole-4-carboxamide+TX, 4-(2-bromo-4-fluoro-phenyl)-N-(2-chloro-6-fluoro-phenyl)-2,5-dimethyl-pyrazol-3-amine+TX, 4-(2-bromo-4-fluorophenyl)-N-(2-chloro-6-fluorophenyl)-1, 3-dimethyl-1H-pyrazol-5-amine+TX, fluindapyr+TX, coumethoxystrobin (jiaxiangjunzhi)+TX, Ivbenmixianan+TX, dichlobentiazox+TX, mandestrobin+TX, 3-(4,4-difluoro-3,4-dihydro-3,3-dimethylisoquinolin-1-yl)quinolone+TX, 2-[2-fluoro-6-[(8-fluoro-2-methyl-3-quinolyl)oxy]phenyl]propan-2-ol+TX, oxathiapiprolin+TX, tert-butyl N-[6-[[[(1-methyltetrazol-5-yl)-phenyl-methylene]amino]oxymethyl]-2-pyridyl]carbamate+TX, pyraziflumid+TX, inpyrfluxam+TX, trolprocarb+TX, mefentrifluconazole+TX, ipfentrifluconazole+TX, 2-(difluoromethyl)-N-[(3R)-3-ethyl-1,1-dimethyl-indan-4-yl]pyridine-3-carboxamide+TX, N′-(2,5-dimethyl-4-phenoxy-phenyl)-N-ethyl-N-methyl-formamidine+TX, N′-[4-(4,5-dichlorothiazol-2-yl)oxy-2,5-dimethyl-phenyl]-N-ethyl-N-methyl-formamidine+TX, [2-[3-[2-[1-[2-[3,5-bis(difluoromethyl)pyrazol-1-yl]acetyl]-4-piperidyl]thiazol-4-yl]-4,5-dihydroisoxazol-5-yl]-3-chloro-phenyl]methanesulfonate+TX, but-3-ynyl N-[6-[[(Z)-[(1-methyltetrazol-5-yl)-phenyl-methylene]amino]oxymethyl]-2-pyridyl]carbamate+TX, methyl N-[[5-[4-(2,4-dimethylphenyl)triazol-2-yl]-2-methyl-phenyl]methyl]carbamate+TX, 3-chloro-6-methyl-5-phenyl-4-(2,4,6-trifluorophenyl)pyridazine+TX, pyridachlometyl+TX, 3-(difluoromethyl)-1-methyl-N-[1,1,3-trimethylindan-4-yl]pyrazole-4-carboxamide+TX, 1-[2-[[1-(4-chlorophenyl)pyrazol-3-yl]oxymethyl]-3-methyl-phenyl]-4-methyl-tetrazol-5-one+TX, 1-methyl-4-[3-methyl-2-[[2-methyl-4-(3,4,5-trimethylpyrazol-1-yl)phenoxy]methyl]phenyl]tetrazol-5-one+TX, aminopyrifen+TX, ametoctradin+TX, amisulbrom+TX, penflufen+TX, (Z,2E)-5-[1-(4-chlorophenyl)pyrazol-3-yl]oxy-2-methoxyimino-N,3-dimethyl-pent-3-enamide+TX, florylpicoxamid+TX, fenpicoxamid+TX, tebufloquin+TX, ipflufenoquin+TX, quinofumelin+TX, isofetamid+TX, N-[2-[2,4-dichloro-phenoxy]phenyl]-3-(difluoromethyl)-1-methyl-pyrazole-4-carboxamide+TX, N-[2-[2-chloro-4-(trifluoromethyl)phenoxy]phenyl]-3-(difluoromethyl)-1-methyl-pyrazole-4-carboxamide+TX, benzothiostrobin+TX, phenamacril+TX, 5-amino-1,3,4-thiadiazole-2-thiol zinc salt (2:1)+TX, fluopyram+TX, flutianil+TX, fluopimomide+TX, pyrapropoyne+TX, picarbutrazox+TX, 2-(difluoromethyl)-N-(3-ethyl-1, 1-dimethyl-indan-4-yl)pyridine-3-carboxamide+TX, 2-(difluoromethyl)-N-((3R)-1,1,3-trimethylindan-4-yl)pyridine-3-carboxamide+TX, 4-[[6-[2-(2,4-difluorophenyl)-1,1-difluoro-2-hydroxy-3-(1,2,4-triazol-1-yl)propyl]-3-pyridyl]oxy]benzonitrile+TX, metyltetraprole+TX, α-(1,1-dimethylethyl)-α-[4′-(trifluoromethoxy) [1, 1′-biphenyl]-4-yl]-5-pyrimidinemethanol+TX, fluoxapiprolin+TX, enoxastrobin+TX, 4-[[6-[2-(2,4-difluorophenyl)-1,1-difluoro-2-hydroxy-3-(1,2,4-triazol-1-yl)propyl]-3-pyridyl]oxy]benzonitrile+TX, 4-[[6-[2-(2,4-difluorophenyl)-1,1-difluoro-2-hydroxy-3-(5-sulfanyl-1,2,4-triazol-1-yl)propyl]-3-pyridyl]oxy]benzonitrile+TX, 4-[[6-[2-(2,4-difluorophenyl)-1,1-difluoro-2-hydroxy-3-(5-thioxo-4H-1,2,4-triazol-1-yl)propyl]-3-pyridyl]oxy]benzonitrile+TX, trinexapac+TX, coumoxystrobin+TX, zhongshengmycin+TX, thiodiazole copper+TX, zinc thiazole+TX, amectotractin+TX, iprodione+TX, N-octyl-N′-[2-(octylamino)ethyl]ethane-1,2-diamine+TX; N′-[5-bromo-2-methyl-6-[(1S)-1-methyl-2-propoxy-ethoxy]-3-pyridyl]-N-ethyl-N-methyl-formamidine+TX, N′-[5-bromo-2-methyl-6-[(1R)-1-methyl-2-propoxy-ethoxy]-3-pyridyl]-N-ethyl-N-methyl-formamidine+TX, N′-[5-bromo-2-methyl-6-(1-methyl-2-propoxy-ethoxy)-3-pyridyl]-N-ethyl-N-methyl-formamidine+TX, N′-[5-chloro-2-methyl-6-(1-methyl-2-propoxy-ethoxy)-3-pyridyl]-N-ethyl-N-methyl-formamidine+TX, N′-[5-bromo-2-methyl-6-(1-methyl-2-propoxy-ethoxy)-3-pyridyl]-N-isopropyl-N-methyl-formamidine+TX (these compounds may be prepared from the methods described in WO2015/155075); N′-[5-bromo-2-methyl-6-(2-propoxypropoxy)-3-pyridyl]-N-ethyl-N-methyl-formamidine+TX (this compound may be prepared from the methods described in IPCOM000249876D); N-isopropyl-N′-[5-methoxy-2-methyl-4-(2,2,2-trifluoro-1-hydroxy-1-phenyl-ethyl)phenyl]-N-methyl-formamidine+TX, N′-[4-(1-cyclopropyl-2,2,2-trifluoro-1-hydroxy-ethyl)-5-methoxy-2-methyl-phenyl]-N-isopropyl-N-methyl-formamidine+TX (these compounds may be prepared from the methods described in WO2018/228896); N-ethyl-N′-[5-methoxy-2-methyl-4-[2-trifluoromethyl)oxetan-2-yl]phenyl]-N-methyl-formamidine+TX, N-ethyl-N′-[5-methoxy-2-methyl-4-[2-trifuoromethyl)tetrahydrofuran-2-yl]phenyl]-N-methyl-formamidine+TX (these compounds may be prepared from the methods described in WO2019/110427); N-[(1R)-1-benzyl-3-chloro-1-methyl-but-3-enyl]-8-fluoro-quinoline-3-carboxamide+TX, N-[(1S)-1-benzyl-3-chloro-1-methyl-but-3-enyl]-8-fluoro-quinoline-3-carboxamide+TX, N-[(1R)-1-benzyl-3,3,3-trifluoro-1-methyl-propyl]-8-fluoro-quinoline-3-carboxamide+TX, N-[(1S)-1-benzyl-3,3,3-trifluoro-1-methyl-propyl]-8-fluoro-quinoline-3-carboxamide+TX, N-[(1R)-1-benzyl-1,3-dimethyl-butyl]-7,8-difluoro-quinoline-3-carboxamide+TX, N-[(1S)-1-benzyl-1,3-dimethyl-butyl]-7,8-difluoro-quinoline-3-carboxamide+TX, 8-fluoro-N-[(1R)-1-[(3-fluorophenyl)methyl]-1.3-dimethyl-butyl]quinoline-3-carboxamide+TX, 8-fluoro-N-[(1S)-1-[(3-fluorophenyl)methyl]-1,3-dimethyl-butyl]quinoline-3-carboxamide+TX, N-[(1R)-1-benzyl-1,3-dimethyl-butyl]-8-fluoro-quinoline-3-carboxamide+TX, N-[(1S)-1-benzyl-1,3-dimethyl-butyl]-8-fluoro-quinoline-3-carboxamide+TX, N-((1R)-1-benzyl-3-chloro-1-methyl-but-3-enyl)-8-fluoro-quinoline-3-carboxamide+TX, N-((1S)-1-benzyl-3-chloro-1-methyl-but-3-enyl)-8-fluoro-quinoline-3-carboxamide+TX (these compounds may be prepared from the methods described in WO2017/153380); 1-(6,7-dimethylpyrazolo[1,5-a]pyridin-3-yl)-4,4,5-trifluoro-3,3-dimethyl-isoquinoline+TX, 1-(6,7-dimethylpyrazolo[1,5-a]pyridin-3-yl)-4,4,6-trifluoro-3,3-dimethyl-isoquinoline+TX, 4,4-difluoro-3,3-dimethyl-1-(6-methylpyrazolo[1,5-a]pyridin-3-yl)isoquinoline+TX, 4,4-difluoro-3,3-dimethyl-1-(7-methylpyrazolo[1,5-a]pyridin-3-yl)isoquinoline+TX, 1-(6-chloro-7-methyl-pyrazolo[1,5-a]pyridin-3-yl)-4,4-difluoro-3,3-dimethyl-isoquinoline+TX (these compounds may be prepared from the methods described in WO2017/025510); 1-(4,5-dimethylbenzimidazol-1-yl)-4,4,5-trifluoro-3,3-dimethyl-isoquinoline+TX, 1-(4,5-dimethylbenzimidazol-1-yl)-4,4-difluoro-3,3-dimethyl-isoquinoline+TX, 6-chloro-4,4-difluoro-3,3-dimethyl-1-(4-methylbenzimidazol-1-yl)isoquinoline+TX, 4,4-difluoro-1-(5-fluoro-4-methyl-benzimidazol-1-yl)-3,3-dimethyl-isoquinoline+TX, 3-(4,4-difluoro-3,3-dimethyl-1-isoquinolyl)-7,8-dihydro-6H-cyclopenta[e]benzimidazole+TX (these compounds may be prepared from the methods described in WO2016/156085); N-methoxy-N-[[4-[5-(trifluoromethyl)-1,2,4-oxadiazol-3-yl]phenyl]methyl]cyclopropanecarboxamide+TX, N,2-dimethoxy-N-[[4-[5-(trifluoromethyl)-1,2,4-oxadiazol-3-yl]phenyl]methyl]propanamide+TX, N-ethyl-2-methyl-N-[[4-[5-(trifluoromethyl)-1,2,4-oxadiazol-3-yl]phenyl]methyl]propanamide+TX, 1-methoxy-3-methyl-1-[[4-[5-(trifluoromethyl)-1,2,4-oxadiazol-3-yl]phenyl]methyl]urea+TX, 1,3-dimethoxy-1-[[4-[5-(trifluoromethyl)-1,2,4-oxadiazol-3-yl]phenyl]methyl]urea+TX, 3-ethyl-1-methoxy-1-[[4-[5-(trifluoromethyl)-1,2,4-oxadiazol-3-yl]phenyl]methyl]urea+TX, N-[[4-[5-(trifluoromethyl)-1,2,4-oxadiazol-3-yl]phenyl]methyl]propanamide+TX, 4,4-dimethyl-2-[[4-[5-(trifluoromethyl)-1,2,4-oxadiazol-3-yl]phenyl]meth yl]isoxazolidin-3-one+TX, 5,5-dimethyl-2-[[4-[5-(trifluoromethyl)-1,2,4-oxadiazol-3-yl]phenyl]methyl]isoxazolidin-3-one+TX, ethyl 1-[[4-[5-(trifluoromethyl)-1,2,4-oxadiazol-3-yl]phenyl]methyl]pyrazole-4-carboxylate+TX, N,N-dimethyl-1-[[4-[5-(trifluoromethyl)-1,2,4-oxadiazol-3-yl]phenyl]methyl]-1,2,4-triazol-3-amine+TX. The compounds in this paragraph may be prepared from the methods described in WO2017/055473, WO2017/055469, WO2017/093348 and WO2017/118689; 2-[6-(4-chlorophenoxy)-2-(trifluoromethyl)-3-pyridyl]-1-(1,2,4-triazol-1-yl)propan-2-ol+TX (this compound may be prepared from the methods described in WO2017/029179); 2-[6-(4-bromophenoxy)-2-(trifluoromethyl)-3-pyridyl]-1-(1,2,4-triazol-1-yl)propan-2-ol+TX (this compound may be prepared from the methods described in WO2017/029179); 3-[2-(1-chlorocyclopropyl)-3-(2-fluorophenyl)-2-hydroxy-propyl]imidazole-4-carbonitrile+TX (this compound may be prepared from the methods described in WO2016/156290); 3-[2-(1-chlorocyclopropyl)-3-(3-chloro-2-fluoro-phenyl)-2-hydroxy-propyl]imidazole-4-carbonitrile+TX (this compound may be prepared from the methods described in WO2016/156290); (4-phenoxyphenyl)methyl 2-amino-6-methyl-pyridine-3-carboxylate+TX (this compound may be prepared from the methods described in WO2014/006945); 2,6-Dimethyl-1H,5H-[1,4]dithiino[2,3-c:5,6-c′]dipyrrole-1,3,5,7(2H,6H)-tetrone+TX (this compound may be prepared from the methods described in WO2011/138281); N-methyl-4-[5-(trifluoromethyl)-1,2,4-oxadiazol-3-yl]benzenecarbothioamide+TX; N-methyl-4-[5-(trifluoromethyl)-1,2,4-oxadiazol-3-yl]benzamide+TX; (Z,2E)-5-[1-(2,4-dichlorophenyl)pyrazol-3-yl]oxy-2-methoxyimino-N,3-dimethyl-pent-3-enamide+TX (this compound may be prepared from the methods described in WO2018/153707); N′-(2-chloro-5-methyl-4-phenoxy-phenyl)-N-ethyl-N-methyl-formamidine+TX; N′-[2-chloro-4-(2-fluorophenoxy)-5-methyl-phenyl]-N-ethyl-N-methyl-formamidine+TX (this compound may be prepared from the methods described in WO2016/202742); 2-(difluoromethyl)-N-[(3S)-3-ethyl-1,1-dimethyl-indan-4-yl]pyridine-3-carboxamide+TX (this compound may be prepared from the methods described in WO2014/095675); (5-methyl-2-pyridyl)-[4-[5-(trifluoromethyl)-1,2,4-oxadiazol-3-yl]phenyl]methanone+TX, (3-methylisoxazol-5-yl)-[4-[5-(trifluoromethyl)-1,2,4-oxadiazol-3-yl]phenyl]methanone+TX (these compounds may be prepared from the methods described in WO2017/220485); 2-oxo-N-propyl-2-[4-[5-(trifluoromethyl)-1,2,4-oxadiazol-3-yl]phenyl]acetamide+TX (this compound may be prepared from the methods described in WO2018/065414); ethyl 1-[[5-[5-(trifluoromethyl)-1,2,4-oxadiazol-3-yl]-2-thienyl]methyl]pyrazole-4-carboxylate+TX (this compound may be prepared from the methods described in WO2018/158365); 2,2-difluoro-N-methyl-2-[4-[5-(trifluoromethyl)-1,2,4-oxadiazol-3-yl]phenyl]acetamide+TX, N-[(E)-methoxyiminomethyl]-4-[5-(trifluoromethyl)-1,2,4-oxadiazol-3-yl]benzamide+TX, N—[(Z)-methoxyiminomethyl]-4-[5-(trifluoromethyl)-1,2,4-oxadiazol-3-yl]benzamide+TX, N—[N-methoxy-C-methyl-carbonimidoyl]-4-[5-(trifluoromethyl)-1,2,4-oxadiazol-3-yl]benzamide+TX (these compounds may be prepared from the methods described in WO2018/202428), chloroinconazide+TX, flumetylsulforim+TX, fluoxytioconazole+TX, flufenoxadiazam+TX, metarylpicoxamid+TX.

The references in brackets behind the active ingredients, e.g. [3878-19-1] refer to the Chemical Abstracts Registry number. The above described mixing partners are known. Where the active ingredients are included in “The Pesticide Manual” [The Pesticide Manual—A World Compendium; Thirteenth Edition; Editor: C. D. S. TomLin; The British Crop Protection Council], they are described therein under the entry number given in round brackets hereinabove for the particular compound; for example, the compound “abamectin” is described under entry number (1). Where “[CCN]” is added hereinabove to the particular compound, the compound in question is included in the “Compendium of Pesticide Common Names”, which is accessible on the internet [A. Wood; Compendium of Pesticide Common Names, Copyright © 1995-2004]; for example, the compound “acetoprole” is described under the internet address http://www.alanwood.net/pesticides/acetoprole.html.

Most of the active ingredients described above are referred to hereinabove by a so-called “common name”, the relevant “ISO common name” or another “common name” being used in individual cases. If the designation is not a “common name”, the nature of the designation used instead is given in round brackets for the particular compound; in that case, the IUPAC name, the IUPAC/Chemical Abstracts name, a “chemical name”, a “traditional name”, a “compound name” or a “development code” is used or, if neither one of those designations nor a “common name” is used, an “alternative name” is employed. “CAS Reg. No” means the Chemical Abstracts Registry Number.

The active ingredient mixture of the compound selected from compounds of formula (I), (I-A), (I-A1), (I-A2), (I-A3), (I-A4), (I-A5), (I-A6), or (I-A7), or compounds selected from compounds listed in Tables C-1 to C-36, or compounds listed in Table P (below), is preferably in a mixing ratio of from 100:1 to 1:100, especially from 50:1 to 1:50, more especially in a ratio of from 20:1 to 1:20, even more especially from 10:1 to 1:10, and still more especially from 5:1 to 1:5 Those mixing ratios are by weight.

The mixtures as described above can be used in a method for controlling pests, which comprises applying a composition comprising a mixture as described above to the pests or their environment, with the exception of a method for treatment of the human or animal body by surgery or therapy and diagnostic methods practised on the human or animal body.

The mixtures comprising a compound selected from compounds of formula (I), (I-A), (I-A1), (I-A2), (I-A3), (I-A4), (I-A5), (I-A6), or (I-A7), or compounds selected from compounds listed in Tables C-1 to C-36, or compounds listed in Table P (below), and one or more active ingredients as described above can be applied, for example, in a single “ready-mix” form, in a combined spray mixture composed from separate formulations of the single active ingredient components, such as a “tank-mix”, and in a combined use of the single active ingredients when applied in a sequential manner, i.e. one after the other with a reasonably short period, such as a few hours or days. The order of applying a compound selected from compounds of formula (I), (I-A), (I-A1), (I-A2), (I-A3), (I-A4), (I-A5), (I-A6), or (I-A7), or compounds selected from compounds listed in Tables C-1 to C-36, or compounds listed in Table P (below), and the active ingredient(s) as described above, is not essential for working the present invention.

The compositions according to the invention can also comprise further solid or liquid auxiliaries, such as stabilizers, for example unepoxidized or epoxidized vegetable oils (for example epoxidized coconut oil, rapeseed oil or soya oil), antifoams, for example silicone oil, preservatives, viscosity regulators, binders and/or tackifiers, fertilizers or other active ingredients for achieving specific effects, for example bactericides, fungicides, nematocides, plant activators, molluscicides or herbicides.

The compositions according to the invention are prepared in a manner known per se, in the absence of auxiliaries for example by grinding, screening and/or compressing a solid active ingredient and in the presence of at least one auxiliary for example by intimately mixing and/or grinding the active ingredient with the auxiliary (auxiliaries). These processes for the preparation of the compositions and the use of the compounds (1) for the preparation of these compositions are also a subject of the invention.

Another aspect of the invention is related to the use of a compound of formula (I) according to the invention or of a preferred individual compound as defined herein, of a composition comprising at least one compound of formula (I) or at least one preferred individual compound as defined herein, or of a fungicidal or insecticidal mixture comprising at least one compound of formula (I) or at least one preferred individual compound as defined herein, in admixture with other fungicides or insecticides as described above, for controlling or preventing infestation of plants, e.g. useful plants such as crop plants, propagation material thereof, e.g. seeds, harvested crops, e.g. harvested food crops, or non-living materials by insects or by phytopathogenic microorganisms, preferably fungal organisms.

A further aspect of invention is related to a method of controlling or preventing an infestation of plants, e.g. useful plants such as crop plants, propagation material thereof, e.g. seeds, harvested crops, e.g. harvested food crops, or of non-living materials by phytopathogenic or spoilage microorganisms or organisms potentially harmful to man, especially fungal organisms, which comprises the application of a compound of formula (I) according to the invention or of a preferred individual compound as defined herein as active ingredient to the plants, to parts of the plants or to the locus thereof, to the propagation material thereof, or to any part of the non-living materials.

Controlling or preventing means reducing infestation by insects or by phytopathogenic or spoilage microorganisms or organisms potentially harmful to man, especially fungal organisms, to such a level that an improvement is demonstrated.

A preferred method of controlling or preventing an infestation of crop plants by phytopathogenic microorganisms, especially fungal organisms, or insects which comprises the application of a compound of formula (I) according to the invention, or an agrochemical composition which contains at least one compound of formula (I), is foliar application. The frequency of application and the rate of application will depend on the risk of infestation by the corresponding pathogen or insect. However, the compounds of formula (I) according to the invention can also penetrate the plant through the roots via the soil (systemic action) by drenching the locus of the plant with a liquid formulation, or by applying the compounds in solid form to the soil, e.g. in granular form (soil application). In crops of water rice such granulates can be applied to the flooded rice field. The compounds of formula (I) may also be applied to seeds (coating) by impregnating the seeds or tubers either with a liquid formulation of the fungicide or coating them with a solid formulation.

A formulation, e.g. a composition containing the compound of formula (I) according to the invention and, if desired, a solid or liquid adjuvant or monomers for encapsulating the compound of formula (I), may be prepared in a known manner, typically by intimately mixing and/or grinding the compound with extenders, for example solvents, solid carriers and, optionally, surface active compounds (surfactants).

Advantageous rates of application are normally from 5 g to 2 kg of active ingredient (a.i.) per hectare (ha), preferably from 10 g to 1 kg a.i./ha, most preferably from 20 g to 600 g a.i./ha. When used as seed drenching agent, convenient dosages are from 10 mg to 1 g of active substance per kg of seeds.

The term “g a.i./ha” as used herein refer to the application rate given in gram [g] of active ingredient [a.i.] per unit of surface [ha]. The unit hectare (symbol ha) is the metric unit of area that equals a square with 100 m side (1 hm²) or 10,000 square meters. Hectare is a commonly used unit of area in the metric system.

When the combinations of the present invention are used fortreating seed, rates of 0.001 to 50 g of a compound of formula (I) per kg of seed, preferably from 0.01 to 10 g per kg of seed are generally sufficient.

Suitably, a composition comprising a compound of formula (I) according to the present invention is applied either preventative, meaning prior to disease development or curative, meaning after disease development.

The compositions of the invention may be employed in any conventional form, for example in the form of a twin pack, a powder for dry seed treatment (DS), an emulsion for seed treatment (ES), a flowable concentrate for seed treatment (FS), a solution for seed treatment (LS), a water dispersible powder for seed treatment (WS), a capsule suspension for seed treatment (CF), a gel for seed treatment (GF), an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a wettable powder (WP) or any technically feasible formulation in combination with agriculturally acceptable adjuvants.

Such compositions may be produced in conventional manner, e.g. by mixing the active ingredients with appropriate formulation inerts (diluents, solvents, fillers and optionally other formulating ingredients such as surfactants, biocides, anti-freeze, stickers, thickeners and compounds that provide adjuvancy effects). Also conventional slow release formulations may be employed where long lasting efficacy is intended. Particularly formulations to be applied in spraying forms, such as water dispersible concentrates (e.g. EC, SC, DC, OD, SE, EW, EO and the like), wettable powders and granules, may contain surfactants such as wetting and dispersing agents and other compounds that provide adjuvancy effects, e.g. the condensation product of formaldehyde with naphthalene sulphonate, an alkylarylsulphonate, a lignin sulphonate, a fatty alkyl sulphate, and ethoxylated alkylphenol and an ethoxylated fatty alcohol.

A seed dressing formulation is applied in a manner known per se to the seeds employing the combination of the invention and a diluent in suitable seed dressing formulation form, e.g. as an aqueous suspension or in a dry powder form having good adherence to the seeds. Such seed dressing formulations are known in the art. Seed dressing formulations may contain the single active ingredients or the combination of active ingredients in encapsulated form, e.g. as slow release capsules or microcapsules.

In general, the formulations include from 0.01 to 90% by weight of active agent, from 0 to 20% agriculturally acceptable surfactant and 10 to 99.99% solid or liquid formulation inerts and adjuvant(s), the active agent consisting of at least the compound of formula (I) according to the invention optionally together with other active agents, particularly microbiocides or conservatives or the like. Concentrated forms of compositions generally contain in between about 2 and 80%, preferably between about 5 and 70% by weight of active agent. Application forms of formulation may for example contain from 0.01 to 20% by weight, preferably from 0.01 to 5% by weight of active agent. Whereas commercial products will preferably be formulated as concentrates, the end user will normally employ diluted formulations.

Whereas it is preferred to formulate commercial products as concentrates, the end user will normally use dilute formulations.

The rates of application vary within wide limits and depend on the nature of the soil, the method of application, the crop plant, the pest to be controlled, the prevailing climatic conditions, and other factors governed by the method of application, the time of application and the target crop. As a general guideline, compounds may be applied at a rate of from 1 to 2000 l/ha, especially from 10 to 1000 l/ha.

Preferred formulations can have the following compositions (weight %):

### Emulsifiable Concentrates:

| | |
|---|---|
| active ingredient: | 1 to 95%, preferably 60 to 90% |
| surface-active agent: | 1 to 30%, preferably 5 to 20% |
| liquid carrier: | 1 to 80%, preferably 1 to 35% |
| Dusts: | |
| active ingredient: | 0.1 to 10%, preferably 0.1 to 5% |
| solid carrier: | 99.9 to 90%, preferably 99.9 to 99% |
| Suspension concentrates: | |
| active ingredient: | 5 to 75%, preferably 10 to 50% |
| water: | 94 to 24%, preferably 88 to 30% |
| surface-active agent: | 1 to 40%, preferably 2 to 30% |
| Wettable powders: | |
| active ingredient: | 0.5 to 90%, preferably 1 to 80% |
| surface-active agent: | 0.5 to 20%, preferably 1 to 15% |
| solid carrier: | 5 to 95%, preferably 15 to 90% |
| Granules: | |
| active ingredient: | 0.1 to 30%, preferably 0.1 to 15% |
| solid carrier: | 99.5 to 70%, preferably 97 to 85% |

The disclosure in the present application makes available each and every combination of embodiments disclosed herein.

The compounds according to the following Tables C-1 to C-36 may be prepared according to the methods described above. The examples which follow are intended to illustrate the invention and show preferred compounds of formula (I). In any of Tables C-1 to C-36 below, the presence of one or more possible asymmetric carbon atoms in a compound of formula (I) according to the invention means that the compounds may occur in chiral isomeric forms, i.e., enantiomeric or diastereomeric forms.

### Table A

This table discloses 22 compounds of formula (A):

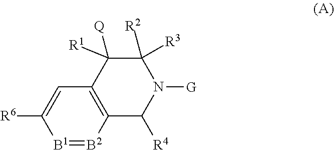

wherein R¹, R², R³, R⁴, R⁶, B¹, B² and Q are as defined above for the compounds of formula (I) and G is of formula

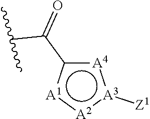

as defined below:

| Index | G |
|---|---|
| G1 | [5-(4-fluorophenyl)-1,2,4-oxadiazole-3- |
| | carbonyl] |
| G2 | [5-(2,4-difluorophenyl)-1,2,4-oxadiazole-3- |
| | carbonyl] |
| G3 | [5-(3,5-difluoro-2-pyridyl)isoxazole-3- |
| | carbonyl] |
| G4 | [5-(3,5-difluoro-2-pyridyl)-1,3,4-thiadiazole- |
| | 2-carbonyl] |
| G5 | [3-(3,5-difluoro-2-pyridyl)-1,2,4-oxadiazole- |
| | 5-carbonyl] |
| G6 | [5-(2-fluorophenyl)isoxazole-3-carbonyl] |
| G7 | [5-(4-fluorophenyl)isoxazole-3-carbonyl] |
| G8 | [5-(2,4-difluorophenyl)isoxazole-3-carbonyl] |
| G9 | [3-(2,4-difluorophenyl)isoxazole-5-carbonyl] |
| G10 | [3-(4-fluorophenyl)isoxazole-5-carbonyl] |
| G11 | [3-(2-fluorophenyl)isoxazole-5-carbonyl] |
| G12 | [5-(2-fluorophenyl)-1,3,4-thiadiazole-2- |
| | carbonyl] |
| G13 | [5-(4-fluorophenyl)-1,3,4-thiadiazole-2- |
| | carbonyl] |
| G14 | [5-(2,4-difluorophenyl)-1,3,4-thiadiazole-2- |
| | carbonyl] |
| G15 | [3-(2,4-difluorophenyl)-1,2,4-oxadiazole-5- |
| | carbonyl] |
| G16 | [3-(4-fluorophenyl)-1,2,4-oxadiazole-5- |
| | carbonyl] |
| G17 | [3-(2-fluorophenyl)-1,2,4-oxadiazole-5- |
| | carbonyl] |
| G18 | [5-(2-fluorophenyl)-1,2,4-oxadiazole-3- |
| | carbonyl] |
| G19 | [5-(3,5-difluoro-2-pyridyl)-1,2,4-oxadiazole- |
| | 3-carbonyl] |
| G20 | [3-(3,5-difluoro-2-pyridyl)isoxazole-5- |
| | carbonyl] |
| G21 | [1-(2,4-difluorophenyl)triazole-4-carbonyl] |
| G22 | [2-(2,4-difluorophenyl)tetrazole-5-carbonyl] |

### Table B

This table discloses 8 compounds of formula (A):

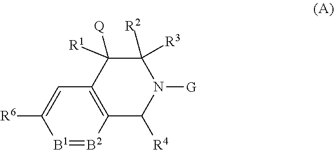

wherein R¹, R², R³, R⁴, R⁵, B¹, and B² are as defined above for the compounds of formula (I) and Q is as defined below:

| Index | Q |
|---|---|
| Q1 | (2,5-dichloro-1-methyl-imidazol-4-yl) |
| Q2 | pyrazin-2-yl |
| Q3 | pyrimidin-5-yl |
| Q4 | (6-methyl-3-pyridyl) |
| Q5 | 4-pyridyl |
| Q6 | 3-pyridyl |
| Q7 | (5-chloro-1-methyl-pyrazol-3-yl) |
| Q8 | 2-pyridyl |

Tables C-1 to C-36 disclose specific compounds of formula (A):

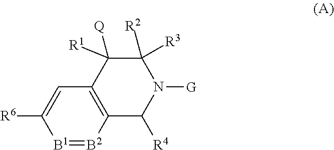

wherein R², R⁴, R⁵ and R⁶ are as defined in any of Tables C-1 to C-36, and G and Q substituents are as defined in Tables A and B, respectively.
Table C-1: This table provides 22 compounds C-1.01 to C-1.22 of formula (A) wherein R¹, R², R³, R⁴ and R⁶ are H, B¹ and B² are C—H, Q is Q¹, and G is as defined in Table A.

For example, compound C-1.01 has the following structure:

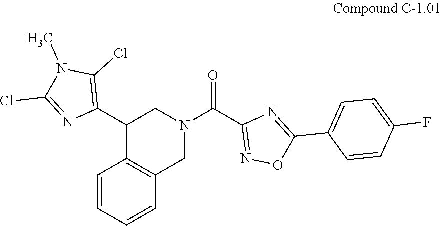 [SMILES: Cn1c(Cl)nc(C2CN(C(=O)c3noc(-c4ccc(F)cc4)n3)Cc3ccccc32)c1Cl]

Table C-2: This table provides 22 compounds C-2.01 to C-2.22 of formula (A) wherein R¹, R², R³, R⁴ and R⁶ are H, B¹ and B² are C—H, Q is Q², and G is as defined in Table A.
Table C-3: This table provides 22 compounds C-3.01 to C-3.22 of formula (A) wherein R¹, R², R³, R⁴ and R⁶ are H, B¹ and B² are C—H, Q is Q³, and G is as defined in Table A.
Table C-4: This table provides 22 compounds C-4.01 to C-4.22 of formula (A) wherein R¹, R², R³, R⁴ and R⁶ are H, B¹ and B² are C—H, Q is Q⁴, and G is as defined in Table A.
Table C-5: This table provides 22 compounds C-5.01 to C-5.22 of formula (A) wherein R¹, R², R³, R⁴ and R⁶ are H, B¹ and B² are C—H, Q is Q⁵, and G is as defined in Table A.
Table C-6: This table provides 22 compounds C-6.01 to C-6.22 of formula (A) wherein R¹, R², R³, R⁴ and R⁶ are H, B¹ and B² are C—H, Q is Q⁶, and G is as defined in Table A.
Table C-7: This table provides 22 compounds C-7.01 to C-7.22 of formula (A) wherein R¹, R², R³, R⁴ and R⁶ are H, B¹ and B² are C—H, Q is Q⁷, and G is as defined in Table A.

For example, compound C-7.22 has the following structure:

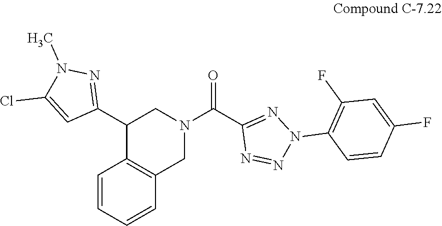 [SMILES: Cn1nc(C2CN(C(=O)c3nnn(-c4ccc(F)cc4F)n3)Cc3ccccc32)cc1Cl]

Table C-8: This table provides 22 compounds C-8.01 to C-8.22 of formula (A) wherein R¹, R², R³, R⁴ and R⁶ are H, B¹ and B² are C—H, Q is Q⁸, and G is as defined in Table A.
Table C-9: This table provides 22 compounds rac-syn-C-9.01 to racemic-syn-C-9.22 of formula (A) wherein R¹, R², R³, and R⁶ are H, R⁴ is CH₃, B¹ and B² are C—H, Q is Q¹, and G is as defined in Table A. In these compounds, the Q substituent and the R⁴ substituent have a syn-relationship to each other.
Table C-10: This table provides 22 compounds racemic-syn-C-10.01 to racemic-syn-C-10.22 of formula (A) wherein R¹, R², R³, and R⁶ are H, R⁴ is CH₃, B¹ and B² are C—H, Q is Q², and G is as defined in Table A. In these compounds, the Q substituent and the R⁴ substituent have a syn-relationship to each other.

For example, compound racemic-syn.C-10.05 has the following structure:

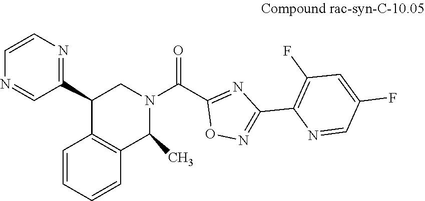 [SMILES: C[C@H]1c2ccccc2[C@@H](c2cnccn2)CN1C(=O)c1nc(-c2ncc(F)cc2F)no1]

Table C-11: This table provides 22 compounds racemic-syn-C-11.01 to racemic-syn-C-11.22 of formula (A) wherein R¹, R², R³, and R⁶ are H, R⁴ is CH₃, B¹ and B² are C—H, Q is Q³, and G is as defined in Table A.
Table C-12: This table provides 22 compounds racemic-syn-C-12.01 to racemic-syn-C-12.22 of formula (A) wherein R¹, R², R³, and R⁶ are H, R⁴ is CH₃, B¹ and B² are C—H, Q is Q⁴, and G is as defined in Table A.
Table C-13: This table provides 22 compounds racemic-syn-C-13.01 to racemic-syn-C-13.22 of formula (A) wherein R¹, R², R³, and R⁶ are H, R⁴ is CH₃, B¹ and B² are C—H, Q is Q⁵, and G is as defined in Table A.
Table C-14: This table provides 22 compounds racemic-syn-C-14.01 to racemic-syn.C-14.22 of formula (A) wherein R¹, R², R³, and R⁶ are H, R⁴ is CH₃, B¹ and B² are C—H, Q is Q⁶, and G is as defined in Table A.
Table C-15: This table provides 22 compounds racemic-syn-C-15.01 to racemic-syn-C-15.22 of formula (A) wherein R¹, R², R³, and R⁶ are H, R⁴ is CH₃, B¹ and B² are C—H, Q is Q⁷, and G is as defined in Table A.
Table C-16: This table provides 22 compounds racemic-syn-C-16.01 to racemic-syn-C-16.22 of formula (A) wherein R¹, R², R³, and R⁶ are H, R⁴ is CH₃, B¹ and B² are C—H, Q is Q⁸, and G is as defined in Table A.
Table C-17: This table provides 22 compounds C-17.01 to C-17.22 of formula (A) wherein R¹ is CH₃, R², R³, R⁴ and R⁶ are H, B¹ and B² are C—H, Q is Q¹, and G is as defined in Table A.
Table C-18: This table provides 22 compounds C-18.01 to C-18.22 of formula (A) wherein R¹ is CH₃, R², R³, R⁴ and R⁶ are H, B¹ and B² are C—H, Q is Q², and G is as defined in Table A.
Table C-19: This table provides 22 compounds C-19.01 to C-19.22 of formula (A) wherein R¹ is CH₃, R², R³, R⁴ and R⁶ are H, B¹ and B² are C—H, Q is Q³, and G is as defined in Table A
Table C-20: This table provides 22 compounds C-20.01 to C-20.22 of formula (A) wherein R¹ is CH₃, R², R³, R⁴ and R⁶ are H, B¹ and B² are C—H, Q is Q⁴, and G is as defined in Table A
Table C-21: This table provides 22 compounds C-21.01 to C-21.22 of formula (A) wherein R¹ is CH₃, R², R³, R⁴ and R⁶ are H, B¹ and B² are C—H, Q is Q⁵, and G is as defined in Table A
Table C-22: This table provides 22 compounds C-22.01 to C-21.22 of formula (A) wherein R¹ is CH₃, R², R³, R⁴ and R⁵ are H, B¹ and B² are C—H, Q is Q⁶, and G is as defined in Table A

For example, compound C-22.03 has the following structure:

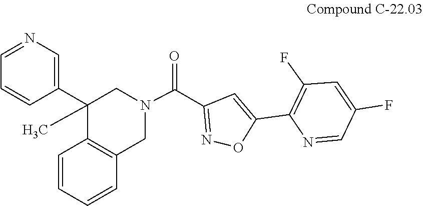 [SMILES: CC1(c2cccnc2)CN(C(=O)c2cc(-c3ncc(F)cc3F)on2)Cc2ccccc21]

Table C-23: This table provides 22 compounds C-23.01 to C-23.22 of formula (A) wherein R¹ is CH₃, R², R³, R⁴ and R⁶ are H, B¹ and B² are C—H, Q is Q⁷, and G is as defined in Table A
Table C-24: This table provides 22 compounds C-24.01 to C-24.22 of formula (A) wherein R¹ is CH₃, R², R³, R⁴ and R⁶ are H, B¹ and B² are C—H, Q is Q⁸, and G is as defined in Table A
Table C-25: This table provides 22 compounds C-25.01 to C-25.22 of formula (A) wherein R¹, R², R³, R⁴ and R⁶ are H, B¹ is CH, B² is C—Cl, Q is Q³, and G is as defined in Table A
Table C-26: This table provides 22 compounds C-26.01 to C-26.22 of formula (A) wherein R¹, R², R³, R⁴ and R⁶ are H, B¹ is CH, B² is C—Cl, Q is Q⁴, and G is as defined in Table A
Table C-27: This table provides 22 compounds C-27.01 to C-27.22 of formula (A) wherein R¹, R², R³, R⁴ and R⁶ are H, B¹ is CH, B² is C—Cl, Q is Q⁵, and G is as defined in Table A
Table C-28: This table provides 22 compounds C-28.01 to C-28.22 of formula (A) wherein R¹, R², R³, R⁴ and R⁶ are H, B¹ is CH, B² is C-Cl. Q is Q⁶, and G is as defined in Table A
Table C-29: This table provides 22 compounds C-29.01 to C-29.22 of formula (A) wherein R¹, R², R³, R⁴ and R⁶ are H, B¹ is CH, B² is C—CN, Q is Q³, and G is as defined in Table A
Table C-30: This table provides 22 compounds C-30.01 to C-30.22 of formula (A) wherein R¹, R², R³, R⁴ and R⁶ are H, B¹ is CH, B² is C—CN, Q is Q⁴, and G is as defined in Table A.

For example, compound 30.14 has the following structure

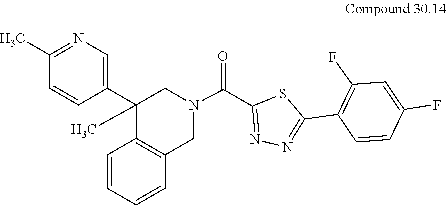 [SMILES: Cc1ccc(C2(C)CN(C(=O)c3nnc(-c4ccc(F)cc4F)s3)Cc3ccccc32)cn1]

Table C-31: This table provides 22 compounds C-31.01 to C-31.22 of formula (A) wherein R¹, R², R³, R⁴ and R⁶ are H, B¹ is CH, B² is C—CN, Q is Q⁵, and G is as defined in Table A
Table C-32: This table provides 22 compounds C-32.01 to C-32.22 of formula (A) wherein R¹, R², R³, R⁴ and R⁶ are H, B¹ is CH, B² is C—CN, Q is Q⁶, and G is as defined in Table A.
Table C-33: This table provides 22 compounds rac-syn-C-33.01 to rac-syn-C-33.22 of formula (A) wherein R¹, R², R³, and R⁶ are H, R⁴ is CH₃, B¹ is CH, B² is C—Cl, Q is Q⁴, and G is as defined in Table A. In these compounds, the Q substituent and the R⁴ substituent have a syn-relationship to each other.
Table C-34: This table provides 22 compounds rac-syn-C-34.01 to rac-syn-C-34.22 of formula (A) wherein R¹, R², R³, and R⁶ are H, R⁴ is CH₃, B¹ is CH, B² is C—Cl, Q is Q⁶, and G is as defined in Table A. In these compounds, the Q substituent and the R⁴ substituent have a syn-relationship to each other.
Table C-35: This table provides 22 compounds rac-syn-C-35.01 to rac-syn-C-35.22 of formula (A) wherein R¹, R², R³, and R⁶ are H, R⁴ is CH₃, B¹ is CH, B² is C—CN, Q is Q⁴, and G is as defined in Table A. In these compounds, the Q substituent and the R⁴ substituent have a syn-relationship to each other.

For example compound rac-syn-C-35.11 has the following structure

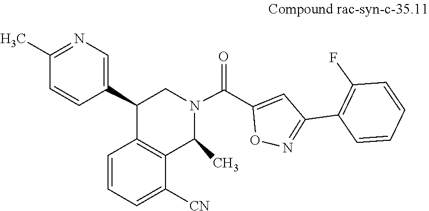 [SMILES: Cc1ccc([C@H]2CN(C(=O)c3cc(-c4ccccc4F)no3)[C@@H](C)c3c(C#N)cccc32)cn1]

Table C-36: This table provides 22 compounds rac-syn-C-36.01 to rac-syn-C-36.22 of formula (A) wherein R¹, R², R³, and R⁶ are H, R⁴ is CH₃, B¹ is CH, B² is C—CN, Q is Q⁶, and G is as defined in Table A. In these compounds, the Q substituent and the R⁴ substituent have a syn-relationship to each other.

### EXAMPLES

The Examples which follow serve to illustrate the invention and are not meant in any way to limit the invention.

The compounds of the invention can be distinguished from known compounds by virtue of greater efficacy at low application rates, which can be verified by a person skilled in the art using the experimental procedures outlined in the Examples, using lower application rates if necessary, for example 60 ppm, 20 ppm or 2 ppm.

Compounds of formula (I) may possess any number of benefits including, inter alia, advantageous levels of biological activity for protecting plants against diseases that are caused by fungi or superior properties for use as agrochemical active ingredients (for example, greater biological activity, an advantageous spectrum of activity, an increased safety profile (including improved crop tolerance), improved physico-chemical properties, or increased biodegradability).

Throughout this description, temperatures are given in degrees Celsius and “m.p.” means melting point. LC/MS means Liquid Chromatography Mass Spectroscopy and the description of the apparatus, and the methods is as follows.

¹H NMR and ¹⁹F NMR measurements were recorded on a Bruker 400 MHz spectrometer, chemical shifts are given in ppm relevant to a TMS (¹H) and CFCl3 (¹⁹F) standard. Spectra measured in deuterated solvents as indicated. Either one of the LCMS methods below was used to characterize the compounds. The characteristic LCMS values obtained for each compound were the retention time (“Rt”, recorded in minutes) and the measured molecular ion (M+H)⁺ or (M−H)⁻.

The following HPLC-MS methods were used for the analysis of the compounds:
Method A (LCMS): Spectra were recorded on a mass spectrometer from Waters (SQD, SQDII Single quadrupole mass spectrometer) equipped with an electrospray source (Polarity: positive and negative ions, Capillary: 3.00 kV, Cone range: 30 V, Extractor: 2.00 V, Source Temperature: 150° C., Desolvation Temperature: 350° C., Cone Gas Flow: 50 I/h, Desolvation Gas Flow: 650 I/h, Mass range: 100 to 900 Da) and an Acquity UPLC from Waters: binary pump, heated column compartment, diode-array detector and ELSD detector. Column: Waters UPLC HSS T3, 1.8 μm, 30×2.1 mm, Temperature: 60° C., DAD Wavelength range (nm): 210 to 500, Solvent Gradient: A=water+5% MeOH+0.05% HCOOH, B=Acetonitrile+0.05% HCOOH, gradient: 10-100% B in 1.2 min; Flow (ml/min): 0.85.
Method B: Spectra were recorded on a Mass Spectrometer from Waters (Acquity QDa Mass Spectrometer) equipped with an electrospray source (Polarity: Positive and Negative Polarity Switch), Capillary: 0.8 kV, Cone range: 25 V, Extractor: V (No extractor voltage for Qda detector) Source Temperature: 120° C., Desolvation Temperature: 600° C., Cone Gas Flow: 50 L/h, Desolvation Gas Flow: 1000 L/h, Mass range: 110 to 850 Da) and an Acquity UPLC from Waters: Quaternary solvent manager, heated column compartment, diode-array detector. Column: Acquity UPLC HSS T3 C18, 1.8 μm, 30×2.1 mm, Temp: 40° C., DAD Wavelength range (nm): 200 to 400, Solvent Gradient: A=water+5% Acetonitrile+0.1% HCOOH, B=Acetonitrile+0.05% HCOOH: gradient: 0 min 10% B; 0.-0.2 min 10-50% B; 0.2-0.6 min 50-100% B; 0.6-1.3 min 100% B; 1.3-1.4 min 100-10% B; 1.4-1.6 min 10% B; Flow (mL/min) 0.6.
Method C: Instrumentation: Mass Spectrometer: 6410 Triple Quadruple Mass Spectrometer from Agilent Technologies; HPLC: Agilent 1200 Series HPLC

Optimized Mass Parameter: lonisation method: Electrospray (ESI); Polarity: Positive and Negative Polarity Switch; Scan Type: MS2 Scan; Capillary (kV): 4.00; Fragmentor (V): 100.00; Gas Temperature (° C.): 350; Gas Flow (L/min): 11; Nebulizer Gas (psi): 40; Mass range: 110 to 1000 Da; Detection (VWD): 254 nm. Optimized Chromatographic Parameter: Gradient conditions: Solvent A: Water with 0.1% formic acid: Acetonitrile: 95:5 v/v; Solvent R: Acetonitrile with 0 10% formic acid

| Time (minutes) | A (%) | B (%) | Flow rate (ml/min) |
|---|---|---|---|
| 0 | 90 | 10 | 1.8 |
| 0.9 | 0 | 100 | 1.8 |
| 1.8 | 0 | 100 | 1.8 |
| 2.2 | 90 | 10 | 1.8 |
| 2.5 | 90 | 10 | 1.8 |

Column: KINETEX EVO C₁₈, Column length: 50 mm, Internal diameter of column: 4.6 mm; Particle Size: 2.6μ. Column oven temperature: 40° C.

FORMULATION EXAMPLES

| Wettable powders | a) | b) | c) |
|---|---|---|---|
| active ingredients | 25% | 50% | 75% |
| sodium lignosulfonate | 5% | 5% | — |
| sodium lauryl sulfate | 3% | — | 5% |
| sodium diisobutylnaphthalenesulfonate | — | 6% | 10% |
| phenol polyethylene glycol ether | — | 2% | — |
| (7-8 mol of ethylene oxide) | | | |
| highly dispersed silicic acid | 5% | 10% | 10% |
| Kaolin | 62% | 27% | — |

The combination is thoroughly mixed with the adjuvants and the mixture is thoroughly ground in a suitable mill, affording wettable powders that can be diluted with water to give suspensions of the desired concentration.

| Powders for dry seed treatment | a) | b) | c) |
|---|---|---|---|
| active ingredients | 25% | 50% | 75% |
| light mineral oil | 5% | 5% | 5% |
| highly dispersed silicic acid | 5% | 5% | — |
| Kaolin | 65% | 40% | — |
| Talcum | — | — | 20% |

The combination is thoroughly mixed with the adjuvants and the mixture is thoroughly ground in a suitable mill, affording powders that can be used directly for seed treatment.

Emulsifiable concentrate

| | |
|---|---|
| active ingredients | 10% |
| octylphenol polyethylene glycol ether (4-5 mol of ethylene oxide) | 3% |
| calcium dodecylbenzene sulfonate | 3% |
| castor oil polyglycol ether (35 mol of ethylene oxide) | 4% |
| Cyclohexanone | 30% |
| xylene mixture | 50% |

Emulsions of any required dilution, which can be used in plant protection, can be obtained from this concentrate by dilution with water.

| Dusts | a) | b) | c) |
|---|---|---|---|
| Active ingredients | 5% | 6% | 4% |
| Talcum | 95% | — | — |
| Kaolin | — | 94% | — |
| mineral filler | — | — | 96% |

Ready-for-use dusts are obtained by mixing the combination with the carrier and grinding the mixture in a suitable mill. Such dusts can also be used for dry dressings for seed.

| Extruder granules | |
|---|---|
| Active ingredients | 15% |
| sodium lignosulfonate | 2% |
| carboxymethylcellulose | 1% |
| Kaolin | 82% |

The combination is mixed and ground with the adjuvants, and the mixture is moistened with water. The mixture is extruded and then dried in a stream of air.

| Coated granules | |
|---|---|
| Active ingredients | 8% |
| polyethylene glycol (mol. wt. 200) | 3% |
| Kaolin | 89% |

The finely ground combination is uniformly applied, in a mixer, to the kaolin moistened with polyethylene glycol. Non-dusty coated granules are obtained in this manner.

Suspension concentrate

| | |
|---|---|
| active ingredients | 40% |
| propylene glycol | 10% |
| nonylphenol polyethylene glycol ether (15 mol of ethylene oxide) | 6% |
| Sodium lignosulfonate | 10% |
| carboxymethylcellulose | 1% |
| silicone oil (in the form of a 75% emulsion in water) | 1% |
| Water | 32% |

The finely ground combination is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

Flowable concentrate for seed treatment

| | |
|---|---|
| active ingredients | 40% |
| propylene glycol | 5% |
| copolymer butanol PO/EO | 2% |
| Tristyrenephenole with 10-20 moles EO | 2% |
| 1,2-benzisothiazolin-3-one (in the form of a 20% | 0.5% |
| solution in water) | |
| monoazo-pigment calcium salt | 5% |
| Silicone oil (in the form of a 75% emulsion in water) | 0.2% |
| Water | 45.3% |

The finely ground combination is intimately mixed with the adjuvants, giving a flowable concentrate from which suspensions of any desired dilution can be obtained by dilution with water, that can be used directly for seed treatment. Using such dilutions, living plants as well as plant propagation material can be treated and protected against infestation by microorganisms, by spraying, pouring or immersion.

### Slow Release Capsule Suspension

28 parts of the combination are mixed with 2 parts of an aromatic solvent and 7 parts of toluene diisocyanate/polymethylene-polyphenylisocyanate-mixture (8:1). This mixture is emulsified in a mixture of 1.2 parts of polyvinylalcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed. The obtained capsule suspension is stabilized by adding 0.25 parts of a thickener and 3 parts of a dispersing agent. The capsule suspension formulation contains 28% of the active ingredients. The medium capsule diameter is 8-15 microns. The resulting formulation is applied to seeds as an aqueous suspension in an apparatus suitable for that purpose.

Formulation types include an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EVW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a wettable powder (WP), a soluble granule (SG) or any technically feasible formulation in combination with agriculturally acceptable adjuvants.

ABBREVIATIONS

| | |
|---|---|
| CDCl₃ | deuterated chloroform |
| DABCO | 1,4-diazabicyclo[2.2.2]octane, also known as |
| | triethylenediamine or TEDA |
| DCC | dicyclohexyl carbodiimide |
| DCM | dichloromethane |
| DMF | dimethylformamide |
| DMSO | dimethyl sulfoxide |
| DMSO-d6 | deuterated Dimethyl sulfoxide |
| EDC | 1-ethyl-3-(3-dimethylaminopropyl)carbodiimide |
| Et₃N | triethylamine |
| EtOAc | ethyl acetate |
| HATU | 1-[bis(dimethylamino)methylene]-1H-1,2,3-triazolo[4,5- |
| | b]pyridinium-3-oxide-hexafluoro phosphate |
| HCl | hydrochloric acid |
| h/hrs | hour/hours |
| LC-MS | Liquid Chromatography Mass Spectrometry (LC-MS or |
| | LCMS) |
| rh | relative humidity |
| rt | room temperature |
| Rt | retention time |
| ssp. | subspecies |
| T3P | propanephosphonic acid anhydride, also called 2,4,6-tripropyl- |
| | 1,3,5,2,4,6-trioxatriphosphorinane-2,4,6-trioxide |
| THF | tetrahydrofuran |

### PREPARATION EXAMPLES

The compounds of formula (I) according to the invention may be prepared using the synthetic techniques described both above and below.

“Mp” means melting point in ° C. Free radicals represent methyl groups. ¹H NMR and ¹⁹F NMR measurements were recorded on a Bruker 400 MHz spectrometer (or 600 MHz as indicated), chemical shifts are given in ppm relevant to a TMS (¹H) and CFCl3 (¹⁹F) standard. Spectra measured in deuterated solvents as indicated. Either one of the LC-MS methods below was used to characterize the compounds. The characteristic LCMS values obtained for each compound were the retention time (“Rt”, recorded in minutes) and the measured molecular ion (M+H)⁺ or (M−H)⁻.

### Example P1: Synthesis of [4-(5-chloro-1,2-dimethyl-imidazol-4-yl)-3,4-dihydro-1H-isoquinolin-2-yl]-(5-phenylisoxazol-3-yl)methanone (Compound P-31, Table P)

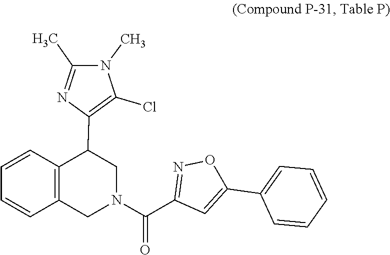 [SMILES: Cc1nc(C2CN(C(=O)c3cc(-c4ccccc4)on3)Cc3ccccc32)c(Cl)n1C]

### Step A: Preparation of 4-(5-chloro-1,2-dimethyl-imidazol-4-yl)isoquinoline

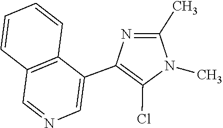 [SMILES: Cc1nc(-c2cncc3ccccc23)c(Cl)n1C]

A solution of 5-chloro-1,2-dimethyl-imidazole (CAS: 937818-18-3, 2.000 g, 14.55 mmol), 4-bromoisoquinoline (CAS: 1532-97-4, 3.277 g, 15.28 mmol) in toluene (58 mL) were treated with cesium carbonate (4.76 g, 14.55 mmol) followed by the 1,10-phenanthroline (0.2649 g, 1.455 mmol) and the mixture degassed with argon for 5 minutes. Palladium(II) acetate (0.3333 g, 1.455 mmol) was then added, and the mixture was stirred for 11 days at 160° C. The reaction mixture was filtered over Celite, and the cake was washed with some EtOAc. The filtrate was concentrated in vacuo, and the crude product obtained was purified by Flash chromatography with EtOAc/cyclohexane to afford 4-(5-chloro-1,2-dimethyl-imidazol-4-yl)isoquinoline.

LC-MS: 258 [M+H], Rt: 0.57 min (Method A)

¹H NMR (400 MHz, CDCl₃) δ ppm 2.57 (s, 3H) 3.68 (s, 3H) 7.78 (td, J=7.63, 1.09 Hz, 1H) 7.90 (ddd, J=8.54, 7.08, 1.45 Hz, 1H) 8.15 (d, J=7.99 Hz, 1H) 8.54 (d, J=8.72 Hz, 1H) 8.66 (s, 1H) 9.32 (s, 1H)

### Step B: Preparation of 4-(5-chloro-1,2-dimethyl-imidazol-4-yl)-1,2,3,4-tetrahydroisoquinoline

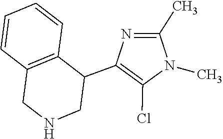 [SMILES: Cc1nc(C2CNCc3ccccc32)c(Cl)n1C]

Sodium cyanoborohydride (0.429 g, 6.60 mmol) was added portionwise to a light-yellow solution of 4-(5-chloro-1,2-dimethyl-imidazol-4-yl)isoquinoline (0.425 g, 1.65 mmol) in methanol (16.5 mL). The resulting light-yellow solution was stirred at rt under argon for 15 minutes. Hydrogen chloride 4N in Dioxane (1.65 mL, 6.60 mmol) was then added dropwise. The resulting brown solution was stirred for 1 hour at rt under argon and the mixture then diluted with water and basified with 2 mL of aqueous NaOH 4N. The reaction mixture was diluted with water and then extracted three times with EtOAc. The combined organic layers, were washed once with brine, dried over anhydrous Na₂SO₄, filtered and concentrated in vacuo to give 4-(5-chloro-1,2-dimethyl-imidazol-4-yl)-1,2,3,4-tetrahydroisoquinoline.

LC-MS: 262 [M+H], Rt: 0.26 min (Method A)

¹H NMR (400 MHz, DMSO d-6) δ ppm 6.98-7.11 (m, 3H) 6.76-6.83 (m, 1H) 3.82-3.99 (m, 3H) 3.47 (s, 3H) 3.01-3.11 (m, 2H) 2.23 (s, 3H).

### Step C: Preparation of [4-(5-chloro-1,2-dimethyl-imidazol-4-yl)-3,4-dihydro-1H-isoquinolin-2-yl]-(5-phenylisoxazol-3-yl)methanone (Compound P-31, Table P)

4-(5-chloro-1,2-dimethyl-imidazol-4-yl)-1,2,3,4-tetrahydroisoquinoline (0.060 g, 0.23 mmol) was dissolved in EtOAc (2.3 mL) and then treated with 1-propanephosphonic anhydride (0.25 mL, 0.41 mmol) followed by the N,N-Diisopropylethylamine (0.12 mL, 0.69 mmol) and then 5-phenylisoxazole-3-carboxylic acid (0.050 g, 0.25 mmol) were added and the resulting mixture was stirred 1 hour at rt under an argon atmosphere. The reaction mixture was diluted with water and EtOAc, the aqueous phase separated, and the aqueous phase back extracted with EtOAc. The combined organic phases were, washed once with brine, dried over anhydrous Na₂SO₄ filtered and concentrated in vacuo. The crude product was purified by Flash chromatography over silica gel eluting with EtOAc to afford [4-(5-chloro-1,2-dimethyl-imidazol-4-yl)-3,4-dihydro-1H-isoquinolin-2-yl]-(5-phenylisoxazol-3-yl)methanone.

LC-MS: 433 [M+H], Rt: 0.85 min (Method A)

### Example P2: Preparation of (5-phenylisoxazol-3-yl)-[4-(3-pyridyl)-3,4-dihydro-1H-isoquinolin-2-yl]methanone (Compound P-6, Table P)

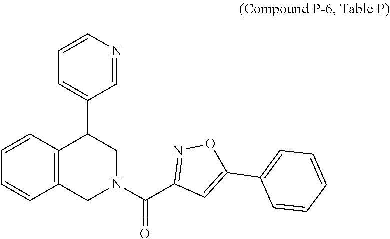 [SMILES: O=C(c1cc(-c2ccccc2)on1)N1Cc2ccccc2C(c2cccnc2)C1]

### Step A: Preparation of 4-(3-pyridyl)isoquinoline

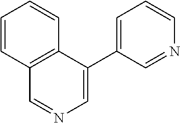 [SMILES: c1cncc(-c2cncc3ccccc23)c1]

Under an Argon atmosphere, 4-bromoisoquinoline (CAS 1532-97-4, 0.550 g, 2.64 mmol) was dissolved in in 1,4-dioxane (8 mL) and water (1.32 mL) and then treated with pyridine-3-boronic acid (CAS: 1692-25-7, 0.502 g, 3.97 mmol), cesium carbonate (2.59 g, 7.93 mmol) and 1,1′-bis(diphenylphosphino)ferrocene-palladium(ii)dichloride dichloromethane complex (0.110 g, 0.132 mmol). The suspension obtained was stirred overnight at 100° C. The reaction was then quenched with water, EtOAc was added, and the aqueous layer was extracted with CHROMABOND® PTL. Isolute® was added to the organic layer and the solvent was removed in vacuo. The crude product was purified by chromatography over silica gel to afford 4-(3-pyridyl)isoquinoline.

LC-MS: 207 [M+H], Rt: 0.48 min (Method A)

¹H NMR (400 MHz, CDCl₃) δ ppm 9.33 (s, 1H) 8.81 (dd, J=2.4, 0.9 Hz, 1H) 8.76 (dd, J=4.8, 1.8 Hz, 1H) 8.51 (s, 1H) 8.06-8.14 (m, 1H) 7.83-7.94 (m, 2H) 7.65-7.79 (m, 2H) 7.45-7.55 (m, 1H)

### Step B: Preparation of 4-(3-pyridyl)-1.2.3.4-tetrahydroisoquinoline

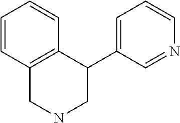 [SMILES: c1cncc(C2CNCc3ccccc32)c1]

A solution of 4-(3-pyridyl)isoquinoline (0.154 g, 0.7468 mmol) in methanol (7.5 mL) was treated with sodium cyanoborohydride (0.2964 g, 4.481 mmol) at room temperature. To this mixture was slowly added hydrochloric acid 4M in Dioxane (0.5 mL, 2 mmol). The solution obtained was stirred for 1 hr at rt and then diluted with water and basified with 2 N NaOH. The MeOH was removed by concentration in vacuo and the remaining aqueous layer was extracted with DCM (three times), The combined organic layers were dried over Na₂SO₄, filtered, and concentrated in vacuo to afford 4-(3-pyridyl)-1,2,3,4-tetrahydroisoquinoline.

LC-MS: 211 [M+H], Rt: 0.22 min (Method A)

¹H NMR (400 MHz, CDCl₃) δ ppm 8.45-8.53 (m, 2H) 7.41 (dt, J=7.8, 2.0 Hz, 1H) 7.19-7.26 (m, 2H) 7.07-7.17 (m, 2H) 6.88 (d, J=7.3 Hz, 1H) 4.08-4.25 (m, 3H) 3.46 (dd, J=13.2, 5.5 Hz, 1H) 3.11 (dd, J=12.8, 6.6 Hz, 1H)

### Step C: Synthesis of (5-phenylisoxazol-3-yl)-[4-(3-pyridyl)-3,4-dihydro-1H-isoquinolin-2-yl]methanone (Compound P-6)

A suspension of 4-(3-pyridyl)-1,2,3,4-tetrahydroisoquinoline (0.161 g, 0.7656 mmol) in DCM (11.4 mL) was cooled to 0° C. and treated with 5-phenylisoxazole-3-carbonyl chloride (0.1621 g, 0.7809 mmol) and triethylamine (0.427 mL, 3.062 mmol). The resulting solution was stirred over night at room temperature and then concentrated in vacuo. The crude product was purified with by combiflash chromatography over silica gel to afford (5-phenylisoxazol-3-yl)-[4-(3-pyridyl)-3,4-dihydro-1H-isoquinolin-2-yl]methanone

LC-MS: 382 [M+H], Rt: 0.91 min (Method A)

¹H NMR (400 MHz, CDCl3) δ ppm 8.37-8.60 (m, 2H) 7.69-7.88 (m, 2H) 7.12-7.57 (m, 8H) 6.88-7.02 (m, 1H) 6.32-6.86 (m, 1H) 4.86-5.30 (m, 2H) 3.92-4.47 (m, 3H)

### Example P3: Preparation of [5-(2,4-difluorophenyl)isoxazol-3-yl]-[4-(1-methyl-1,2,4-triazol-3-yl)-3,4-dihydro-1H-isoquinolin-2-yl]methanone (Compound P-33, Table P)

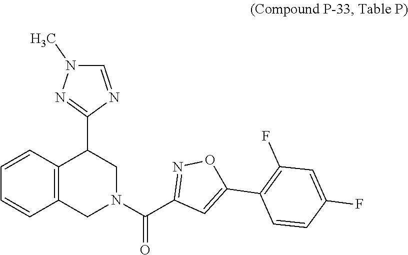 [SMILES: Cn1cnc(C2CN(C(=O)c3cc(-c4ccc(F)cc4F)on3)Cc3ccccc32)n1]

### Step A: Preparation of 4-(1-methyl-1.2.4-triazol-3-yl)isoquinoline

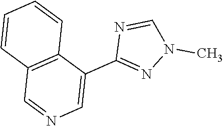 [SMILES: Cn1cnc(-c2cncc3ccccc23)n1]

A Microwave vial was charged with 3-bromo-1-methyl-1,2,4-triazole (0.12 g, 0.74 mmol), 4-isoquinolylboronic acid (0.19 g, 1.1 mmol), sodium carbonate (0.24 g, 2.2 mmol), 1,4-dioxane (2.2 mL) and water (0.74 mL). The reaction mixture was degassed with nitrogen for 5 min and then treated with chloro(2-dicyclohexylphosphino-2′,4′,6′-triisopropyl-1,1′-biphenyl)[2-(2′-amino-1,1′-biphenyl)]palladium(II) (0.030 g, 0.037 mmol). The resulting reaction mixture was stirred at 120° C. for 2 h under microwave irradiation (which turned to dark brown color). Progress of reaction was monitoring by LCMS and TLC. Upon completion, the reaction mixture was cooled to RT and diluted with saturated aqueous NH₄Cl (50 mL). The mixture was extracted with EtOAc (2×50 mL), and the combined organic layers were washed with brine, dried over sodium sulfate, and concentrated in vacuo. The crude product was purified by combi flash chromatography to afford 4-(1-methyl-1,2,4-triazol-3-yl)isoquinoline.

LC-MS (Method B) retention time 0.46 min m/z 211 (M+H)

1H NMR (400 MHz, CDCl₃d) δ ppm 9.29 (s, 1H), 9.20 (s, 1H), 9.04 (d, J=8.56 Hz, 1H), 8.22 (s, 1H), 8.03 (d, J=8.19 Hz, 1H), 7.80 (ddd, J=8.47, 7.00, 1.28 Hz, 1H), 7.64-7.67 (m, 1H), 4.07 (s, 3H).

### Step B: Preparation of 4-(1-methyl-1,2,4-triazol-3-yl)-1,2,3,4-tetrahydroisoquinoline

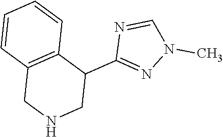 [SMILES: Cn1cnc(C2CNCc3ccccc32)n1]

A round bottom flask, equipped with a magnetic stirrer bar, was charged with 4-(1-methyl-1,2,4-triazol-3-yl)isoquinoline (0.09 g, 0.4 mmol), methanol (5 mL) and sodium cyanoborohydride (0.2 g, 3 mmol). Then, hydrochloric acid 1.25 M in methanol (0.9 mL, 1 mmol) was added dropwise at room temperature (gas evolution) and the mixture was stirred at room temperature for 12 h. The reaction mixture was poured into water (50 mL) and basified with 2M NaOH (pH 12). The mixture was extracted with EtOAc (3×30 mL), and the combined organic layers were then washed with brine, dried over Na₂SO₄ and concentrated in vacuo. The crude compound 4-(1-methyl-1,2,4-triazol-3-yl)-1,2,3,4-tetrahydroisoquinoline (0.08 g) used as such in next step.

LC-MS (Method C) retention time 0.16 min, m/z 215 (M+H)

### Step C: Preparation of [5-(2,4-difluorophenyl)isoxazol-3-yl]-[4-(1-methyl-1,2,4-triazol-3-yl)-3,4-dihydro-1H-isoquinolin-2-yl]methanone (Compound P-33, Table P)

[5-(2,4-difluorophenyl)isoxazol-3-yl]-[4-(1-methyl-1,2,4-triazol-3-yl)-3,4-dihydro-1H-isoquinolin-2-yl]methanone was prepared analogously to Example 1 Step C.

LC-MS: 422 [M+H], Rt: 1.07 min (Method B)

¹H NMR (400 MHz, CDCl₃) δ ppm 3.85 (s, 3H) 3.91 (s, 1H) 4.30-4.37 (m, 2H) 4.48-4.60 (m, 3H) 4.99 (d, J=17.48 Hz, 1H) 5.12 (d, J=17.36 Hz, 1H) 5.20 (d, J=7.82 Hz, 1H) 6.84 (d, J=3.91 Hz, 1H) 6.97-7.26 (m, 8H) 7.92-8.03 (m, 3H)

### Example P4: Preparation of 4-[2-[5-(2,4-difluorophenyl)isoxazole-3-carbonyl]-3,4-dihydro-1H-isoquinolin-4-yl]-1,5-dimethyl-pyrrole-2-carbonitrile (compound P-20, Table P)

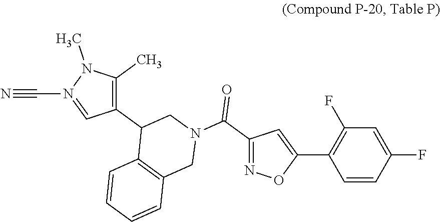

### Step A: Preparation of 4-bromo-1,5-dimethyl-pyrrole-2-carbonitrile

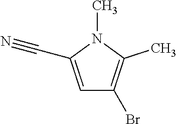

To a stirred solution of 1,5-dimethylpyrrole-2-carbonitrile (3.00 g, 25.0 mmol) in acetic acid (30 mL) was added Bromine (1.32 mL, 25.5 mmol) dropwise, and the resulting reaction mixture was stirred at room temperature for 16 h. The reaction mixture was diluted with water and quenched with a saturated aqueous solution of sodium thiosulphate. The mixture was extracted with EtOAc (three times), and the combined organic layers were washed with sodium bicarbonate solution Na₂SO₄ and concentrated in vacuo, The crude product was further purified over a silica gel column to give 4-bromo-1,5-dimethyl-pyrrole-2-carbonitrile.

¹H NMR (400 MHz, CDCl₃) δ ppm 6.75 (s, 1H), 3.68 (s, 3H), 2.26 (s, 3H)

### Step B: Preparation of 4-(4-isoquinolyl)-1,5-dimethyl-pyrrole-2-carbonitrile

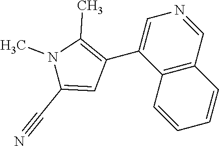 [SMILES: Cc1c(-c2cncc3ccccc23)cc(C#N)n1C]

A Microwave vial was charged with 4-bromo-1,5-dimethyl-pyrrole-2-carbonitrile (1.00 g, 5.02 mmol), 4-isoquinolylboronic acid (1.30 g, 7.54 mmol), sodium carbonate (1.60 g, 15.1 mmol), 1,4-dioxane (15.1 mL) and water (5 mL). The reaction mixture was degassed with nitrogen for 5.0 min and then treated with chloro(2-dicyclohexylphosphino-2′,4′,6′-triisopropyl-1,1′-biphenyl)[2-(2′-amino-1,1′-biphenyl)]palladium(II) (0.20 g, 0.251 mmol). The resulting reaction mixture was stirred at 120° C. for 2 h in a MW and the progress of reaction monitored by LCMS and TLC. After completion of the reaction, the mixture was cooled to room temperature and diluted with NH₄Cl sat. (50 mL). The mixture was extracted with EtOAc (2×50 mL), and the combined organic layers were washed with brine, dried over Na₂SO₄ and concentrated in vacuo. The crude purified over column chromatography to afford 4-(4-isoquinolyl)-1,5-dimethyl-pyrrole-2-carbonitrile.

LC-MS retention time 1.00 min m/z 248 (M+H) (Method C)

### Step C: Preparation of 1,5-dimethyl-4-(1,2,3,4-tetrahydroisoquinolin-4-yl)pyrrole-2-carbonitrile

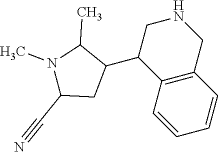 [SMILES: CC1C(C2CNCc3ccccc32)CC(C#N)N1C]

A round bottom flask was charged with 4-(4-isoquinolyl)-1,5-dimethyl-pyrrole-2-carbonitrile (0.90 μm, 3.64 mmol), methanol (18.20 mL) and sodium cyanoborohydride (2.40 g, 36.4 mmol). Then, hydrochloric acid (4.0 mol/L) in dioxane (21.8 mL) was added dropwise at room temperature (gas evolution) and the mixture was stirred at room temperature for 5 h. Reaction was monitored by LCMS. After completion of reaction, reaction mixture was basified by adding sat NaHCO₃ solution and then extracted with EtOAc (three times). The combined organic layers were concentrated in vacuo to yield 1,5-dimethyl-4-(1,2,3,4-tetrahydroisoquinolin-4-yl)pyrrole-2-carbonitrile which was used without further purification used the next step

LC-MS retention time 0.91 min m/z 252 (M+H); (Method C)

### Step D: Preparation of 4-[2-[5-(2,4-difluorophenyl)isoxazole-3-carbonyl]-3,4-dihydro-1H-isoquinolin-4-yl]-1,5-dimethyl-pyrrole-2-carbonitrile (compound P-20)

The desired product was prepared analogously to Example 1 Step C to afford 4-[2-[5-(2,4-difluorophenyl)isoxazole-3-carbonyl]-3,4-dihydro-1H-isoquinolin-4-yl]-1,5-dimethyl-pyrrole-2-carbonitrile

LC-MS: 459 [M+H], Rt: 1.22 min (Method B)

¹H NMR (400 MHz, CDCl₃) δ ppm 0.86-0.91 (m, 1H) 1.18-1.36 (m, 3H) 1.23-1.32 (m, 2H) 2.16 (s, 3H) 2.31 (s, 1H) 3.54 (s, 3H) 3.63-3.67 (m, 1H) 3.69 (s, 1H) 3.97 (dd, J=12.84, 6.72 Hz, 1H) 4.18-4.27 (m, 3H) 4.37 (dd, J=12.65, 4.71 Hz, 1H) 4.97-5.03 (m, 1H) 5.06-5.12 (m, 1H) 5.24 (s, 1H) 5.27-5.29 (m, 1H) 5.30-5.32 (m, 1H) 6.24 (s, 1H) 6.36 (s, 1H) 6.71 (d, J=3.67 Hz, 1H) 6.97-7.09 (m, 5H) 7.12-7.25 (m, 3H) 7.28-7.31 (m, 1H) 7.92-8.01 (m, 1H)

### Example P5: Preparation of [5-(2,4-difluorophenyl)isoxazol-3-yl]-[4-methyl-4-(3-pyridyl)-1,3-dihydroisoquinolin-2-yl]methanone (Compound P-27. Table P)

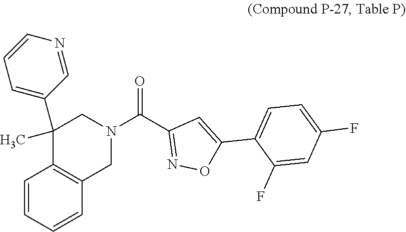 [SMILES: CC1(c2cccnc2)CN(C(=O)c2cc(-c3ccc(F)cc3F)on2)Cc2ccccc21]

### Step 1: Preparation of 2-phenyl-2-(3-pyridyl)propanenitrile

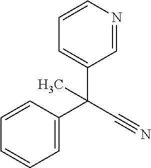 [SMILES: CC(C#N)(c1ccccc1)c1cccnc1]

A sealed tube, equipped with a magnetic stirrer bar, was charged with 3-fluoropyridine (1.78 g, 18.37 mmol), 2-phenylpropanenitrile (2.41 g, 18.373 mmol), potassium tert-butoxide (2.10 g, 18.373 mmol) and DMSO (20 mL) under an argon atmosphere. The reaction mixture was stirred 2 hours at 80° C. The reaction mixture was then poured into an aqueous solution saturated with potassium bicarbonate. The water phase was extracted with EtOAc (2×50 mL), and the combined organic layers dried over Na₂SO₄ and concentrated in vacuo. The crude material was purified by combiflash (silica gel, gradient: EtOAc in cyclohexane) to afford the title compound as a yellow liquid.

LC-MS (Method A): 209 [M+H]+; retention time: 0.82 min.

¹H NMR (400 MHz, CDCl₃) δ ppm 2.17 (s, 3H) 7.26-7.5 (m, 6H) 7.72 (d, 1H) 8.6 (d, 1H) 8.68 (d, 1H)

### Step 2: Preparation of 2-phenyl-2-(3-pyridyl)propan-1-amine

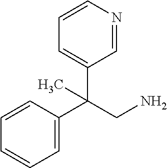 [SMILES: CC(CN)(c1ccccc1)c1cccnc1]

A 100 mL-3-necked flask, equipped with a magnetic stirrer bar, was charged with 2-phenyl-2-(3-pyridyl)propanenitrile (prepared as described above in step 1, 1.10 g, 5.76 mmol) and THF (18 mL). To this yellow solution borane dimethyl sulfide complex (1.74 mL, 17.28 mmol) was added dropwise at room temperature under an argon atmosphere and the resulting colorless mixture was stirred at 65° C. for 2 hours. The reaction mixture was cooled to 0° C. before adding hydrochloric acid (3.86 mL, 23.16 mmol) dropwise (strong gas evolution) and the mixture was stirred at 65° C. for 1 hour and then allowed to cool down to room temperature. The mixture was diluted with water (80 mL), basified with 13 mL 6 M NaOH (pH 12) and then extracted twice with EtOAc (3×50 mL). The combined organic layers were washed with brine, dried over Na₂SO₄, and concentrated in vacuo to give the title compound as an orange gum which was used without further purification in the next step.

LC-MS (Method A): 213 [M+H]+; retention time: 0.21 min.

### Step 3: Preparation of methyl N-[2-phenyl-2-(3-pyridyl)propyl]carbamate

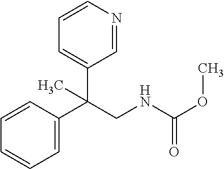 [SMILES: COC(=O)NCC(C)(c1ccccc1)c1cccnc1]

A one necked 250 mL round bottom flask, equipped with a magnetic stirrer bar, was charged with 2-phenyl-2-(3-pyridyl)propan-1-amine (prepared as described above in step 2, 1.55 g, 7.30 mmol) and DCM (30 mL). Methyl chloroformate (0.68 mL, 8.76 mmol), followed by triethylamine (3.07 mL, 21.9 mmol) were added dropwise at 0-10° C. under an argon atmosphere. The ice-bath was removed, and the mixture was stirred at room temperature for 1 hour. The reaction mixture was poured into water and the organic phase was separated. The water phase was extracted twice with EtOAc and the combined organic layers were dried over Na₂SO₄ and concentrated in vacuo. The crude material was purified by combiflash (silica gel, gradient: ethyl acetate in cyclohexane) to afford the title compound as a colourless gum.

LC-MS (Method A): 271 [M+H]+; retention time: 0.58 min.

### Step 4: Preparation of methyl 4-methyl-4-(3-pyridyl)-1,3-dihydroisoquinoline-2-carboxylate

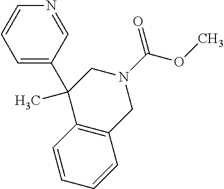 [SMILES: COC(=O)N1Cc2ccccc2C(C)(c2cccnc2)C1]

A one necked round bottom flask, equipped with a magnetic stirrer bar, was charged with methyl N-[2-phenyl-2-(3-pyridyl)propyl]carbamate (prepared as described above in step 3, 180 mg, 0.665 mmol), hydrochloric acid (5.00 mL/mmol, 4.00 g, 3.33 mL, 40.50 mmol) and paraformaldehyde (39.96 mg, 0.42 mmol). The mixture was stirred at room temperature for 40 minutes, where LC-MS analysis showed reaction completion. The reaction mixture was slowly poured into water (30 mL), neutralized with NaHCO₃, and extracted with EtOAc (3×20 mL). The combined organic layers were washed with brine, dried over Na₂SO₄, and concentrated in vacuo. The crude material was purified by combiflash (silica gel, gradient: ethyl acetate in cyclohexane) to afford the title compound as a colourless gum.

LC-MS (Method A): 283 [M+H]+; retention time: 0.67 min.

### Step 5: Preparation of 4-methyl-4-(3-pyridyl)-2.3-dihydro-1H-isoquinoline

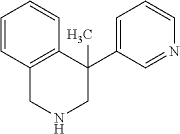 [SMILES: CC1(c2cccnc2)CNCc2ccccc21]

A one necked round bottom flask, equipped with a magnetic stirrer bar, was charged with methyl 4-methyl-4-(3-pyridyl)-1,3-dihydroisoquinoline-2-carboxylate (prepared as described above in step 4, 120 mg, 0.42 mmol), 1,2-dichloroethane (5.00 mL/mmol, 2.12 ml) and iodotrimethylsilane (263.1 mg, 0.18 mL, 1.27 mmol). The mixture was stirred at 60° C. under argon atmosphere for 45 min, where LC-MS analysis showed almost full conversion. The reaction was then allowed to stir overnight at room temperature. The reaction mixture was slowly poured into water, neutralized with NaHCO₃, and extracted twice with EtOAc. The combined organic layers were washed with brine, dried over Na₂SO₄, and concentrated in vacuo to yield the title compound as a dark orange gum which was used without further purification in the next step.

LC-MS (Method A): 225 [M+H]+; retention time: 0.16 min.

### Step 6: Preparation of [5-(2,4-difluorophenyl)isoxazol-3-yl]-[4-methyl-4-(3-pyridyl)-1,3-dihydroisoquinolin-2-yl]methanone (Compound P-27, Table P)

A one necked round bottom flask, equipped with a magnetic stirrer bar, was charged with 4-methyl-4-(3-pyridyl)-2,3-dihydro-1H-isoquinoline (prepared as described above in step 5, 60 mg, 0.267 mmol), EtOAc (2.675 mL, 27.30 mmol), N.N-diisopropylethylamine (104 mg, 0.138 mL, 0.8025 mmol) and 5-(2,4-difluorophenyl)isoxazole-3-carboxylic acid (68.30 mg, 0.2942 mmol). Then, T3P (510.70 mg, 0.473 mL, 0.8025 mmol) was added dropwise at RT and the mixture was stirred at RT for 60 min. The reaction mixture was slowly poured into water, neutralized with NaHCO₃, and extracted twice with EtOAc. The combined organic layers were washed with brine, dried over sodium sulfate, and concentrated in vacuo. The crude material was purified by combiflash (silica gel, gradient: ethyl acetate in cyclohexane) to afford the title compound as a white gum.

LC-MS (Method A): 432 [M+H]+; retention time: 0.95 min.

Further examples of synthesized compounds of formula (I) are shown in Table P.

TABLE P
Synthesized compounds and Spectral and Physical Chemical Data.

| | | | | [M + | | |
|---|---|---|---|---|---|---|
| | | | | H] | | |
| | | | RT | (mea- | Meth- | MP |
| Entry | IUPAC name | Structure | (min) | sured) | od | [° C.] |
| [P-1 | [5-(2,4- difluorophenyl)isoxazol-3- yl]-[8-fluoro-4-(3-pyridyl)- 3,4-dihydro-1H-isoquinolin- 2-yl]methanone | 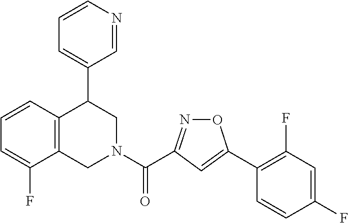 [SMILES: O=C(c1cc(-c2ccc(F)cc2F)on1)N1Cc2c(F)cccc2C(c2cccnc2)C1] | | | | 115-120 |
| P-2 | [5-(2,4- difluorophenyl)isoxazol-3- yl]-4-[4-(4-pyridyl)-3,4- dihydro-1H-isoquinolin-2- yl]methanone | 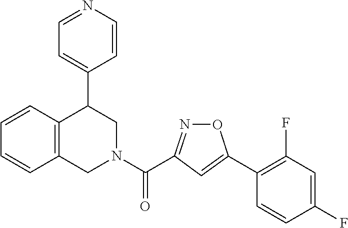 [SMILES: O=C(c1cc(-c2ccc(F)cc2F)on1)N1Cc2ccccc2C(c2ccncc2)C1] | | | | 130-135 |
| P-3 | [5-(2,4- difluorophenyl)isoxazol-3- yl]-[4-(6-methyl-3-pyridyl)- 3,4-dihydro-1H-isoquinolin- 2-yl]methanone | 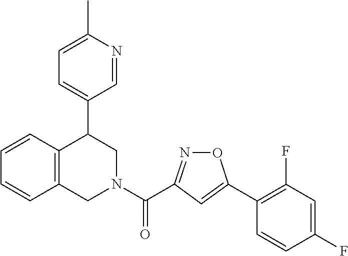 [SMILES: Cc1ccc(C2CN(C(=O)c3cc(-c4ccc(F)cc4F)on3)Cc3ccccc32)cn1] | | | | 65-70 |
| P-4 | [5-(2,4- difluorophenyl)isoxazol-3- yl]-(4-pyrimidin-5-yl-3,4- dihydro-1H-isoquinolin-2- yl)methanone | 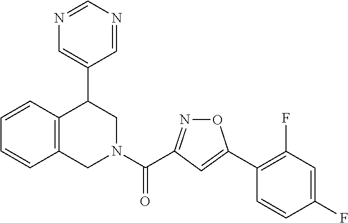 [SMILES: O=C(c1cc(-c2ccc(F)cc2F)on1)N1Cc2ccccc2C(c2cncnc2)C1] | | | | 97-98 |
| P-5 | [5-(2,4- difluorophenyl)isoxazol-3- yl]-[4-(3-pyridyl)-3,4- dihydro-1H-isoquinolin-2- yl]methanone | 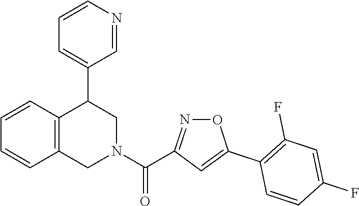 [SMILES: O=C(c1cc(-c2ccc(F)cc2F)on1)N1Cc2ccccc2C(c2cccnc2)C1] | 0.94 | 418 | A | |
| P-6 | (5-phenylisoxazol-3-yl)-[4- (3-pyridyl)-3,4-dihydro-1H- isoquinolin-2-yl]methanone | 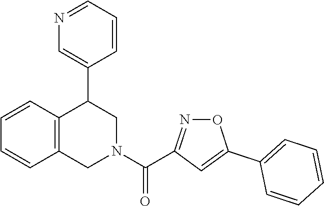 [SMILES: O=C(c1cc(-c2ccccc2)on1)N1Cc2ccccc2C(c2cccnc2)C1] | 0.91 | 382 | A | |
| P-7 | [4-(5-chloro-1-methyl- pyrazol-3-yl)-3,4-dihydro- 1H-isoquinolin-2-yl]-[5-(2,4- difluorophenyl)isoxazol-3- yl]methanone | 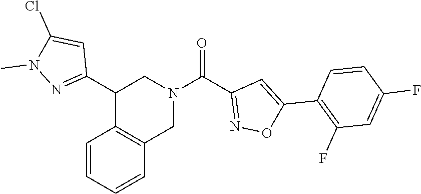 [SMILES: Cn1nc(C2CN(C(=O)c3cc(-c4ccc(F)cc4F)on3)Cc3ccccc32)cc1Cl] | 1.15 | 455 | A | |
| P-8 | [5-(2,4- difluorophenyl)isoxazol-3- yl]-(4-pyrazin-2-yl-3,4- dihydro-1H-isoquinolin-2- yl]methanone | 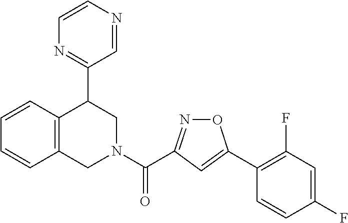 [SMILES: O=C(c1cc(-c2ccc(F)cc2F)on1)N1Cc2ccccc2C(c2cnccn2)C1] | | | | 105-110 |
| P-9 | [5-(2,4- difluorophenyl)isoxazol-3- yl]-[5-fluoro-4-(3-pyridyl)- 3,4-dihydro-1H-isoquinolin- 2-yl]methanone | 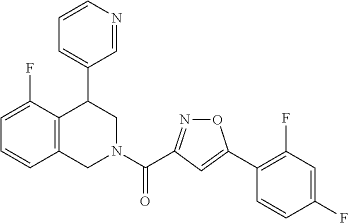 [SMILES: O=C(c1cc(-c2ccc(F)cc2F)on1)N1Cc2cccc(F)c2C(c2cccnc2)C1] | | | | |
| P-10 | [4-(2,5-dichloro-1-methyl- imidazol-4-yl)-3,4-dihydro- 1H-isoquinolin-2-yl]-[5-(2,4- difluorophenyl)isoxazol-3- yl]methanone | 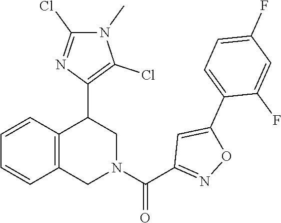 [SMILES: Cn1c(Cl)nc(C2CN(C(=O)c3cc(-c4ccc(F)cc4F)on3)Cc3ccccc32)c1Cl] | 1.15 | 491 | A | |
| P-11 | [4-(2-chloro-1,5-dimethyl- imidazol-4-yl)-3,4-dihydro- 1H-isoquinolin-2-yl]-[5-(2,4- difluorophenyl)-1,3,4- thiadiazol-2-yl]methanone | 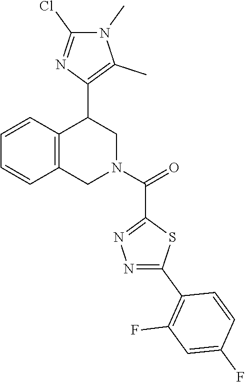 [SMILES: Cc1c(C2CN(C(=O)c3nnc(-c4ccc(F)cc4F)s3)Cc3ccccc32)nc(Cl)n1C] | 1.07 | 486 | A | |
| P-12 | [5-(2,4- difluorophenyl)isoxazol-3- yl]-[4-(6-fluoro-3-pyridyl)- 3,4-dihydro-1H-isoquinolin- 2-yl]methanone | 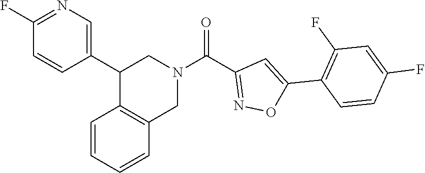 [SMILES: O=C(c1cc(-c2ccc(F)cc2F)on1)N1Cc2ccccc2C(c2ccc(F)nc2)C1] | 1.09 | 436 | A | |
| P-13 | [5-(2,4- difluorophenyl)isoxazol-3- yl]-[4-(2-methyl-3-pyridyl)- 3,4-dihydro-1H-isoquinolin- 2-yl]methanone | 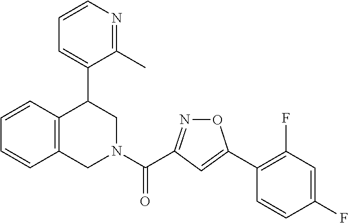 [SMILES: Cc1ncccc1C1CN(C(=O)c2cc(-c3ccc(F)cc3F)on2)Cc2ccccc21] | | | | 115-120 |
| P-14 | [5-(2,4- difluorophenyl)isoxazol-3- yl]-[4-(4-methyl-3-pyridyl)- 3,4-dihydro-1H-isoquinolin- 2-yl]methanone | 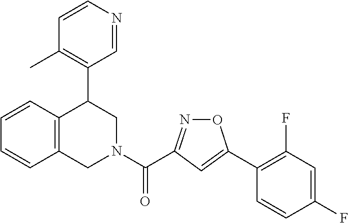 [SMILES: Cc1ccncc1C1CN(C(=O)c2cc(-c3ccc(F)cc3F)on2)Cc2ccccc21] | | | | 140-145 |
| P-15 | [5-(2,4- difluorophenyl)isoxazol-3- yl]-[4-(2-pyridyl)-3,4- dihydro-1H-isoquinolin- 2-yl]methanone | 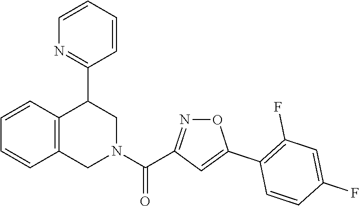 [SMILES: O=C(c1cc(-c2ccc(F)cc2F)on1)N1Cc2ccccc2C(c2ccccn2)C1] | 1.05 | 418 | A | |
| P-16 | [4-(2-chloro-1,5-dimethyl- imidazol-4-yl)-3,4-dihydro- 1H-isoquinolin-2-yl]-[5-(2,4- difluorophenyl)isoxazol-3- yl]methanone | 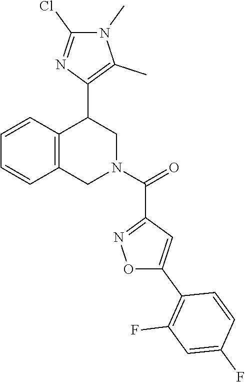 [SMILES: Cc1c(C2CN(C(=O)c3cc(-c4ccc(F)cc4F)on3)Cc3ccccc32)nc(Cl)n1C] | 1.07 | 469 | A | |
| P-17 | [4-(5-chloro-1,2-dimethyl- imidazol-4-yl)-3,4-dihydro- 1H-isoquinolin-2-yl]-[5-(2,4- difluorophenyl)isoxazol-3- yl]methanone | 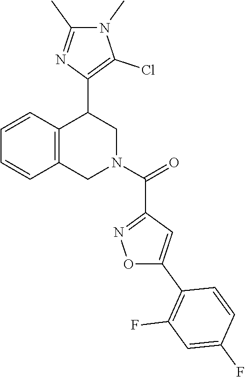 [SMILES: Cc1nc(C2CN(C(=O)c3cc(-c4ccc(F)cc4F)on3)Cc3ccccc32)c(Cl)n1C] | 0.86 | 469 | A | |
| P-18 | 4-[2-[5-(4- fluorophenyl)isoxazole-3- carbonyl]-3,4-dihydro-1H- isoquinolin-4-yl]-1,5- dimethyl-pyrrole-2- carbonitrile | 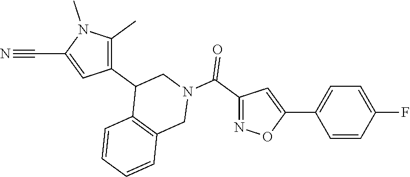 [SMILES: Cc1c(C2CN(C(=O)c3cc(-c4ccc(F)cc4)on3)Cc3ccccc32)cc(C#N)n1C] | 1.19 | 441 | B | 100-103 |
| P-19 | 4-[2-[3-(2,4- difluorophenyl)isoxazole-5- carbonyl]-3,4-dihydro-1H- isoquinolin-4-yl]-1,5- dimethyl-pyrrole-2- carbonitrile | 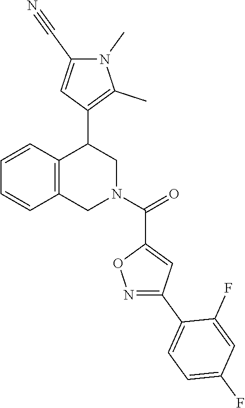 [SMILES: Cc1c(C2CN(C(=O)c3cc(-c4ccc(F)cc4F)no3)Cc3ccccc32)cc(C#N)n1C] | 1.20 | 459 | B | 85-88 |
| P-20 | 4-[2-[5-(2,4- difluorophenyl)isoxazole-3- carbonyl]-3,4-dihydro-1H- isoquinolin-4-yl]-1,5- dimethyl-pyrrole-2- carbonitrile | 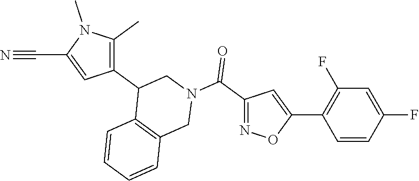 [SMILES: Cc1c(C2CN(C(=O)c3cc(-c4ccc(F)cc4F)on3)Cc3ccccc32)cc(C#N)n1C] | 1.22 | 459 | B | 79-82 |
| P-21 | [4-(5-bromo-1-methyl- pyrazol-3-yl)-3,4-dihydro- 1H-isoquinolin-2-yl]-[5-(2,4- difluorophenyl)isoxazol-3- yl]methanone | 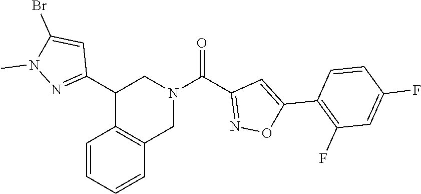 [SMILES: Cn1nc(C2CN(C(=O)c3cc(-c4ccc(F)cc4F)on3)Cc3ccccc32)cc1Br] | 1.14 | 499-501 | A | |
| P-22 | [5-(2,4- difluorophenyl)isoxazol-3- yl]-[4-(5-methyl-3-pyridyl)- 3,4-dihydro-1H-isoquinolin- 2-yl]methanone | 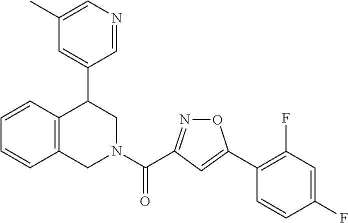 [SMILES: Cc1cncc(C2CN(C(=O)c3cc(-c4ccc(F)cc4F)on3)Cc3ccccc32)c1] | | | | 75-80 |
| P-23 | [4-(5-chloro-6-methyl-3- pyridyl)-3,4-dihydro-1H- isoquinolin-2-yl]-[5-(2,4- difluorophenyl)isoxazol-3- yl]methanone | 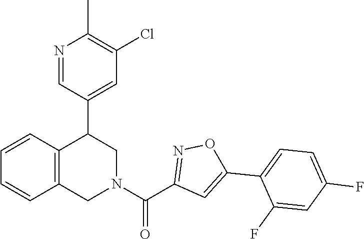 [SMILES: Cc1ncc(C2CN(C(=O)c3cc(-c4ccc(F)cc4F)on3)Cc3ccccc32)cc1Cl] | 1.16 | 466 | A | |
| P-24 | [4-(4-chloro-3-pyridyl)-3,4- dihydro-1H-isoquinolin-2- yl]-[5-(2,4- difluorophenyl)isoxazol-3- yl]methanone | 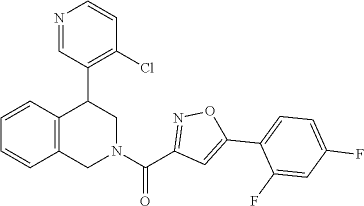 [SMILES: O=C(c1cc(-c2ccc(F)cc2F)on1)N1Cc2ccccc2C(c2cnccc2Cl)C1] | 1.10 | 452 | A | |
| P-25 | [5-(2,4- difluorophenyl)isoxazol-3- yl]-[4-(5-methyl-1,2,4- oxadiazol-3-yl)-3,4-dihydro- 1H-isoquinolin- 2-yl]methanone | 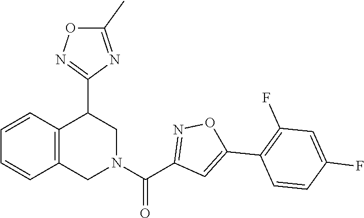 [SMILES: Cc1nc(C2CN(C(=O)c3cc(-c4ccc(F)cc4F)on3)Cc3ccccc32)no1] | 1.14 | 423 | B | |
| P-26 | 1,5-dimethyl-4-[2-(5- phenylisoxazole-3- carbonyl)-3,4-dihydro-1H- isoquinolin-4-yl]pyrrole-2- carbonitrile | 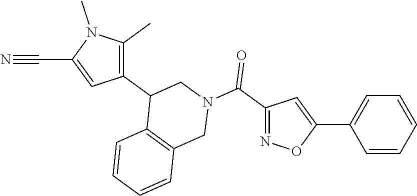 [SMILES: Cc1c(C2CN(C(=O)c3cc(-c4ccccc4)on3)Cc3ccccc32)cc(C#N)n1C] | 1.44 | 423 | C | 93-96 |
| P-27 | [5-(2,4- difluorophenyl)isoxazol-3- yl]-[4-methyl-4-(3-pyridyl)- 1,3-dihydroisoquinolin-2- yl]methanone | 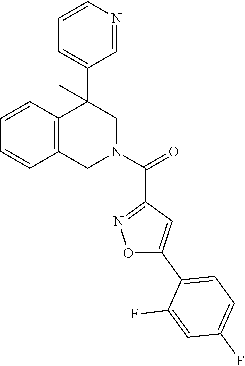 [SMILES: CC1(c2cccnc2)CN(C(=O)c2cc(-c3ccc(F)cc3F)on2)Cc2ccccc21] | 0.95 | 432 | A | |
| P-28 | 4-[2-(5-(2,4-difluorophenyl)- 1,3,4-thiadiazole-2- carbonyl]-3,4-dihydro-1H- isoquinolin-4-yl]-1,5- dimethyl-pyrrole-2- carbonitrile | 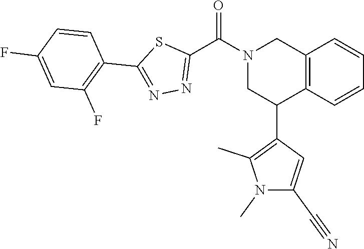 [SMILES: Cc1c(C2CN(C(=O)c3nnc(-c4ccc(F)cc4F)s3)Cc3ccccc32)cc(C#N)n1C] | 1.23 | 476 | B | 220-223 |
| P-29 | [4-(5-chloro-6-methyl-3- pyridyl)-3,4-dihydro-1H- isoquinolin-2-yl]-(5- phenylisoxazol-3- yl)methanone | 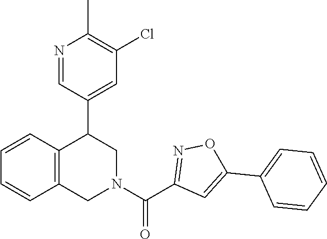 [SMILES: Cc1ncc(C2CN(C(=O)c3cc(-c4ccccc4)on3)Cc3ccccc32)cc1Cl] | 1.13 | 430 | A | |
| P-30 | [4-(4-chloro-3-pyridyl)-3,4- dihydro-1H-isoquinolin-2- yl]-(5-phenylisoxazol-3- yl)methanone | 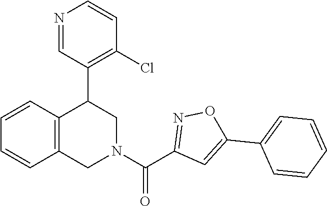 [SMILES: O=C(c1cc(-c2ccccc2)on1)N1Cc2ccccc2C(c2cnccc2Cl)C1] | 1.07 | 416 | A | |
| P-31 | [4-(5-chloro-1,2-dimethyl- imidazol-4-yl)-3,4-dihydro- 1H-isoquinolin-2-yl]-(5- phenylisoxazol-3- yl)methanone | 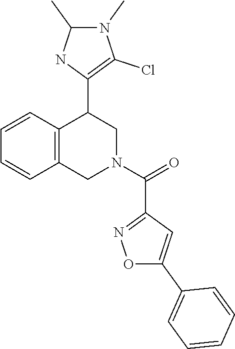 [SMILES: CC1NC(C2CN(C(=O)c3cc(-c4ccccc4)on3)Cc3ccccc32)=C(Cl)N1C] | 0.85 | 433 | A | |
| P-32 | [4-methyl-4-(3-pyridyl)-1,3- dihydroisoquinolin-2-yl]-(5- phenylisoxazol-3- yl)methanone | 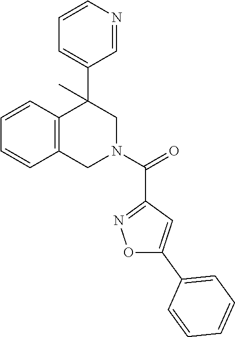 [SMILES: CC1(c2cccnc2)CN(C(=O)c2cc(-c3ccccc3)on2)Cc2ccccc21] | 0.92 | 396 | A | |
| P-33 | [5-(2,4- difluorophenyl)isoxazol-3- yl]-[4-(1-methyl-1,2,4- triazol-3-yl)-3,4-dihydro-1H- isoquinolin-2-yl]methanone | 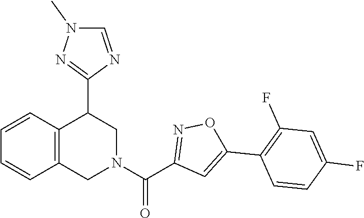 [SMILES: Cn1cnc(C2CN(C(=O)c3cc(-c4ccc(F)cc4F)on3)Cc3ccccc32)n1] | 1.07 | 422 | B | |
| P-34 | [4-(6-fluoro-3-pyridyl)-3,4- dihydro-1H-isoquinolin-2- yl]-(5-phenylisoxazol-3- yl)methanone | 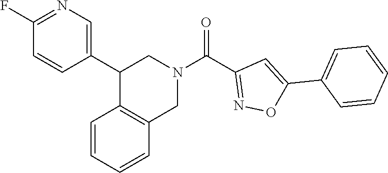 [SMILES: O=C(c1cc(-c2ccccc2)on1)N1Cc2ccccc2C(c2ccc(F)nc2)C1] | 1.07 | 400 | A | |
| P-35 | [5-(2,4- difluorophenyl)isoxazol-3- yl]-[4-(1-methyltriazol-4-yl)- 3,4-dihydro-1H-isoquinolin- 2-yl]methanone | 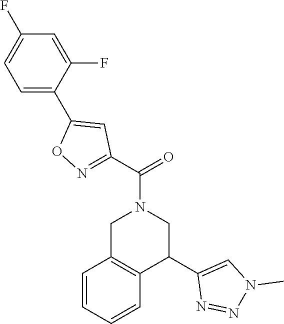 [SMILES: Cn1cc(C2CN(C(=O)c3cc(-c4ccc(F)cc4F)on3)Cc3ccccc32)nn1] | 0.96 | 422 | A | |
| P-36 | [5-(2,4- difluorophenyl)isoxazol-3- yl]-(4-pyridazin-3-yl-3,4- dihydro-1H-isoquinolin-2- yl)methanone | 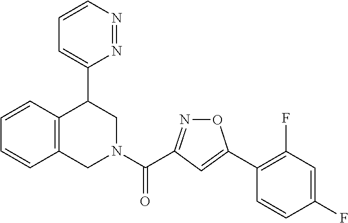 [SMILES: O=C(c1cc(-c2ccc(F)cc2F)on1)N1Cc2ccccc2C(c2cccnn2)C1] | | | | 110-115 |
| P-37 | [4-(6-chloro-3-pyridyl)-3,4- dihydro-1H-isoquinolin-2- yl]-[5-(2,4- difluorophenyl)isoxazol-3- yl]methanone | 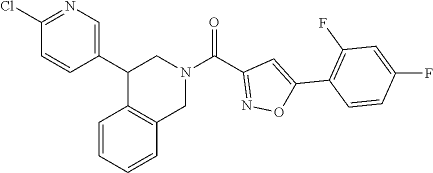 [SMILES: O=C(c1cc(-c2ccc(F)cc2F)on1)N1Cc2ccccc2C(c2ccc(Cl)nc2)C1] | 1.12 | 452 | A | |
| P-38 | [4-(5-chloro-3-pyridyl)-3,4- dihydro-1H-isoquinolin-2- yl]-[5-(2,4- difluorophenyl)isoxazol-3- yl]methanone | 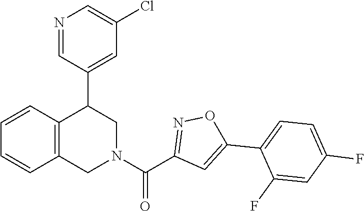 [SMILES: O=C(c1cc(-c2ccc(F)cc2F)on1)N1Cc2ccccc2C(c2cncc(Cl)c2)C1] | 1.12 | 452 | A | |
| P-39 | [5-(2,4- difluorophenyl)isoxazol-3- yl]-[4-(1,5-dimethyl-1,2,4- triazol-3-yl)-3,4-dihydro-1H- isoquinolin-2-yl]methanone | 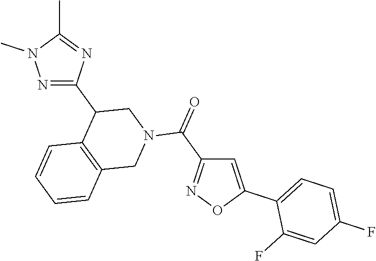 [SMILES: Cc1nc(C2CN(C(=O)c3cc(-c4ccc(F)cc4F)on3)Cc3ccccc32)nn1C] | 1.05 | 436 | B | 150-152 |
| P-40 | [5-(2,4- difluorophenyl)isoxazol-3- yl]-[5-(3-pyridyl)-6,8- dihydro-5H-1,7- naphthyridin-7- yl]methanone | 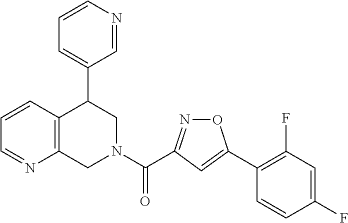 [SMILES: O=C(c1cc(-c2ccc(F)cc2F)on1)N1Cc2ncccc2C(c2cccnc2)C1] | | | | 120-125 |

### BIOLOGICAL EXAMPLES

### Example B1: Alternaria Solani/Tomato/Leaf Disc (Early Blight)

Tomato leaf disks cv. Baby are placed on agar in multiwell plates (24-well format) and sprayed with the formulated test compound diluted in water. The leaf disks are inoculated with a spore suspension of the fungus 2 days after application. The inoculated leaf disks are incubated at 23° C./21° C. (day/night) and 80% rh under a light regime of 12/12 h (light/dark) in a climate cabinet and the activity of a compound is assessed as percent disease control compared to untreated when an appropriate level of disease damage appears on untreated check disk leaf disks (5 to 7 days after application).

The following compounds gave at least 80% control of Alternaria solani at 200 ppm when compared to untreated control under the same conditions, which showed extensive disease development: P-13, P-35

### Example B2: Botryotinia fuckeliana (Botrytis cinerea)/Liquid Culture (Gray Mould)

Conidia of the fungus from cryogenic storage are directly mixed into nutrient broth (Vogels broth). After placing a (DMSO) solution of test compound into a microtiter plate (96-well format), the nutrient broth containing the fungal spores is added. The test plates are incubated at 24° C. and the inhibition of growth is determined photometrically 3 to 4 days after application.

The following compounds gave at least 80% control of Botryotinia fuckeliana at 20 ppm when compared to untreated control under the same conditions, which showed extensive disease development: P-1, P-6

### Example B3: Glomerella lagenarium (Colletotrichum lagenarium)/Liquid Culture (Anthracnose)

Conidia of the fungus from cryogenic storage are directly mixed into nutrient broth (PDB potato dextrose broth). After placing a (DMSO) solution of test compound into a microtiter plate (96-well format), the nutrient broth containing the fungal spores is added. The test plates are incubated at 24° C. and the inhibition of growth is measured photometrically 3 to 4 days after application.

The following compounds gave at least 80% control of Glomerella lagenarium at 20 ppm when compared to untreated control under the same conditions, which showed extensive disease development: P-1, P-2, P-3, P-4, P-5

### Example B4: Phaeosphaeria Nodorum (Septoria nodorum)/Wheat/Leaf Disc Preventative (Glume Blotch)

Wheat leaf segments cv. Kanzler are placed on agar in a multiwell plate (24-well format) and sprayed with the formulated test compound diluted in water. The leaf disks are inoculated with a spore suspension of the fungus 2 days after application. The inoculated test leaf disks are incubated at 20° C. and 75% rh under a light regime of 12 h light/12 h darkness in a climate cabinet and the activity of a compound is assessed as percent disease control compared to untreated when an appropriate level of disease damage appears in untreated check leaf disks (5 to 7 days after application).

The following compounds gave at least 80% control of Phaeosphaeria nodorum at 200 ppm when compared to untreated control under the same conditions, which showed extensive disease development: P-7

### Example B5: Monographella nivalis (Microdochium nivale)/Liquid Culture (Foot Rot Cereals)

Conidia of the fungus from cryogenic storage are directly mixed into nutrient broth (PDB potato dextrose broth). After placing a (DMSO) solution of test compound into a microtiter plate (96-well format), the nutrient broth containing the fungal spores is added. The test plates are incubated at 24° C. and the inhibition of growth is determined photometrically 4 to 5 days after application.

The following compounds gave at least 80% control of Monographella nivalis at 20 ppm when compared to untreated control under the same conditions, which showed extensive disease development: P-1, P-2, P-3, P-4, P-5, P-6, P-7, P-8, P-9, P-12, P-15

### Example B6: Mycosphaerella arachidis (Cercospora arachidicola)/Liquid Culture (Early Leaf Spot)

Conidia of the fungus from cryogenic storage are directly mixed into nutrient broth (PDB potato dextrose broth). After placing a (DMSO) solution of test compound into a microtiter plate (96-well format), the nutrient broth containing the fungal spores is added. The test plates are incubated at 24° C. and the inhibition of growth is determined photometrically 4 to 5 days after application.

The following compounds gave at least 80% control of Mycosphaerella arachidis at 20 ppm when compared to untreated control under the same conditions, which showed extensive disease development: P-1, P-3, P-4, P-5, P-6, P-7, P-8, P-9, P-10, P-11, P-13, P-14

### Example B7: Puccinia recondita f. Sp. Tritici/Wheat/Leaf Disc Curative (Brown Rust)

Wheat leaf segments cv. Kanzler are placed on agar in multiwell plates (24-well format). The leaf segments are inoculated with a spore suspension of the fungus. Plates are stored in darkness at 19° C. and 75% rh. The formulated test compound diluted in water is applied 1 day after inoculation. The leaf segments are incubated at 19° C. and 75% rh under a light regime of 12 h light/12 h darkness in a climate cabinet and the activity of a compound is assessed as percent disease control compared to untreated when an appropriate level of disease damage appears in untreated check leaf segments (6 to 8 days after application).

The following compounds gave at least 80% control of Puccinia recondita f sp. tritici at 200 ppm when compared to untreated control under the same conditions, which showed extensive disease development: P-19

### Example B8: Magnaporthe grisea (Pyricularia oryzae)/Rice/Leaf Disc Preventative (Rice Blast)

Rice leaf segments cv. Ballila are placed on agar in a multiwell plate (24-well format) and sprayed with the formulated test compound diluted in water. The leaf segments are inoculated with a spore suspension of the fungus 2 days after application. The inoculated leaf segments are incubated at 22° C. and 80% rh under a light regime of 24 h darkness followed by 12 h light/12 h darkness in a climate cabinet and the activity of a compound is assessed as percent disease control compared to untreated when an appropriate level of disease damage appears in untreated check leaf segments (5 to 7 days after application).

The following compounds gave at least 80% control of Magnaporthe grisea at 200 ppm when compared to untreated control under the same conditions, which showed extensive disease development: P-13, P-30

### Example B9: Pyrenophora teres/Barley/Leaf Disc Preventative (Net Blotch)

Barley leaf segments cv. Hasso are placed on agar in a multiwell plate (24-well format) and sprayed with the formulated test compound diluted in water. The leaf segments are inoculated with a spore suspension of the fungus 2 days after application. The inoculated leaf segments are incubated at 20° C. and 65% rh under a light regime of 12 h light/12 h darkness in a climate cabinet and the activity of a compound is assessed as disease control compared to untreated when an appropriate level of disease damage appears in untreated check leaf segments (5 to 7 days after application).

The following compounds gave at least 80% control of Pyrenophora teres at 200 ppm when compared to untreated control under the same conditions, which showed extensive disease development: P-1

### Example B10: Pythium ultimum/Liquid Culture (Seedling Damping Off)

Mycelia fragments and oospores of a newly grown liquid culture of the fungus are directly mixed into nutrient broth (PDB potato dextrose broth). After placing a (DMSO) solution of test compound into a microtiter plate (96-well format), the nutrient broth containing the fungal mycelia/spore mixture is added. The test plates are incubated at 24° C. and the inhibition of growth is determined photometrically 2 to 3 days after application.

The following compounds gave at least 80% control of Pythium ultimum at 20 ppm when compared to untreated control under the same conditions, which showed extensive disease development: P-2

### Example B11: Thanatephorus cucumeris (Rhizoctonia solani)/Liquid Culture (Foot Rot, Damping-Off)

Mycelia fragments of a newly grown liquid culture of the fungus are directly mixed into nutrient broth (PDB potato dextrose broth). After placing a (DMSO) solution of the test compounds into a microtiter plate (96-well format), the nutrient broth containing the fungal material is added. The test plates are incubated at 24° C. and the inhibition of growth is determined photometrically 3 to 4 days after application.

The following compounds gave at least 80% control of Thanatephorus cucumeris at 20 ppm when compared to untreated control under the same conditions, which showed extensive disease development: P-2

### Example B12: Mycosphaerella graminicola (Septoria tritici)/Liquid Culture (Septoria Blotch)

Conidia of the fungus from cryogenic storage are directly mixed into nutrient broth (PDB potato dextrose broth). After placing a (DMSO) solution of test compound into a microtiter plate (96-well format), the nutrient broth containing the fungal spores is added. The test plates are incubated at 24° C. and the inhibition of growth is determined photometrically 4 to 5 days after application.

The following compounds gave at least 80% control of Mycosphaerella graminicola at 20 ppm when compared to untreated control under the same conditions, which showed extensive disease development:
P-1, P-2, P-3, P-4, P-5, P-6, P-7, P-8, P-9, P-10, P-11, P-12, P-13, P-14, P-15, P-16, P-17, P-18, P-19, P-20, P-21, P-22, P-23, P-24, P-25, P-26, P-27, P-28, P-29, P-30, P-31, P-32, P-33, P-34, P-35, P-36, P-39

1. A compound of formula (I) 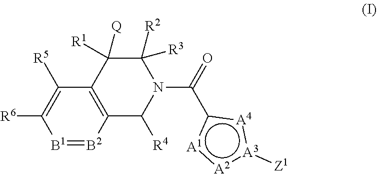
wherein
Q is selected from a 5- or 6-membered heteroaryl; wherein any of said 5- or 6-membered heteroaryl contains 1, 2, 3 or 4 heteroatoms independently selected from N, O or S, with the proviso that no more than one is O or S; and wherein any of said 5- or 6-membered heteroaryl is unsubstituted or substituted with 1, 2 or 3 substituents independently selected from halogen, cyano, C₁-C₄-alkyl, C₁-C₄-haloalkyl, or C₃-C₆cycloalkyl;
R¹ is selected from hydrogen, halogen, C₁-C₄haloalkyl, C₃-C₆ cycloalkyl, or C₁-C₄ alkyl;
R² and R³ are independently selected from hydrogen, or C₁-C₄-alkyl;
R⁴ is selected from hydrogen, C₁-C₄-alkyl, C₁-C₄-alkylcarbonyl, N—C₁-C₄-alkoxy-C—C₁-C₄ alkyl-carbonimidoyl, N-hydroxy-C—C₁-C₄-alkyl-carbonimidoyl, C₁-C₄-alkoxycarbonyl, N-methoxy-N-methyl-carbonyl, C₁-C₄ alkylaminocarbonyl, di(C₁-C₄ alkyl)aminocarbonyl, phenyl, a 5- to 6-membered heteroaryl, or C₃-C₆-cycloalkyl;
wherein any of said 5- or 6-membered heteroaryl contains 1, 2, 3 or 4 heteroatoms independently selected from N, O or S, with the proviso that no more than one is O or S;
wherein any of said phenyl and 5- to 6-membered heteroaryl are unsubstituted or substituted with 1, 2 or 3 substituents independently selected from halogen, cyano, C₁-C₄-alkyl, C₁-C₄-haloalkyl, carboxy, or C₁-C₄-alkoxy; and wherein said C₃-C₆-cycloalkyl is unsubstituted or substituted with 1, 2, or 3 substituents independently selected from halogen, cyano, C₁-C₄alkyl, C₁-C₄haloalkyl, or C₁-C₄alkoxy;
B¹ is selected from CR⁷, or N;
B² is selected from CR⁸, or N;
R⁵, R⁶, R⁷ and R⁸ are independently selected from hydrogen, halogen, C₁-C₄ alkyl, C₁-C₄ haloalkyl, C₁-C₄ alkoxy, C₁-C₄haloalkoxy, C₂-C₄alkenyloxy, C₂-C₄ alkynyloxy, C₁-C₄ alkylsulfanyl, C₁-C₄ alkylsulfinyl, C₁-C₄ alkylsulfonyl, C₁-C₄ alkoxy-C₁-C₄ alkyl, N—C₁-C₄alkylamino, N,N-di(C₁-C₄alkyl)amino, C₁-C₄ alkoxycarbonyl, C₁-C₄ alkylcarbonyl, N—C₁-C₄ alkoxy-C₁-C₄ alkyl-carbonimidoyl, N-hydroxy-C₁-C₄ alkyl-carbonimidoyl, hydroxy, trifluoromethylsulfonyloxy, cyano, carboxy, amino, phenyl, 5- or 6-membered heteroaryl, or C₃-C₆ cycloalkyl; wherein any of said 5- or 6-membered heteroaryl contains 1, 2, 3 or 4 heteroatoms independently selected from N, O or S, with the proviso that no more than one is O or S; and wherein any of said phenyl, 5- or 6-membered heteroaryl and C₃-C₆-cycloalkyl are unsubstituted or substituted with 1, 2 or 3 substituents independently selected from halogen, cyano, C₁-C₄ alkyl, C₁-C₄haloalkyl, or C₁-C₄alkoxy;
A¹, A², A⁴ are independently selected from methine, N, NR¹⁰, O, or S, with the proviso that at least one of A¹, A² and A⁴ is selected from N, NR¹⁰, O or S, and that no more than one of A¹, A² and A⁴ is O or S, wherein R¹⁰ is selected from hydrogen or C₁-C₄ alkyl;
A³ is C or N with the proviso that when A³ is N, A¹, A² and A⁴ are methine, N or NR¹⁰ wherein R¹⁰ is selected from hydrogen or C₁-C₄ alkyl; and
Z¹ is selected from C₁-C₄ alkyl, phenyl, 5- or 6-membered heteroaryl, or C₃-C₆ cycloalkyl; wherein any of said 5- or 6-membered heteroaryl contains 1, 2, 3, or 4 heteroatoms independently selected from N, O, or S, with the proviso that no more than one is O or S; wherein any of said phenyl, and 5- or 6-membered heteroaryl are unsubstituted or substituted by 1, 2, or 3 substituents independently selected from halogen, cyano, C₁-C₄ alkyl, C₂-C₄ alkenyl, C₁-C₄ haloalkyl, C₁-C₄ alkoxy, C₁-C₄ haloalkoxy, C₁-C₄ alkylsulfanyl, C₁-C₄ alkylsulfinyl, C₁-C₄ alkylsulfonyl, or C₂-C₄ alkynyl; and wherein said C₃-C₆ cycloalkyl is unsubstituted or substituted by 1, 2, or 3 substituents independently selected from halogen, cyano, C₁-C₄ alkyl, C₁-C₄ haloalkyl, or C₁-C₄alkoxy;
or an agrochemically acceptable salt, stereoisomer, or N-oxide thereof, with the proviso that the compound of formula (I) is not 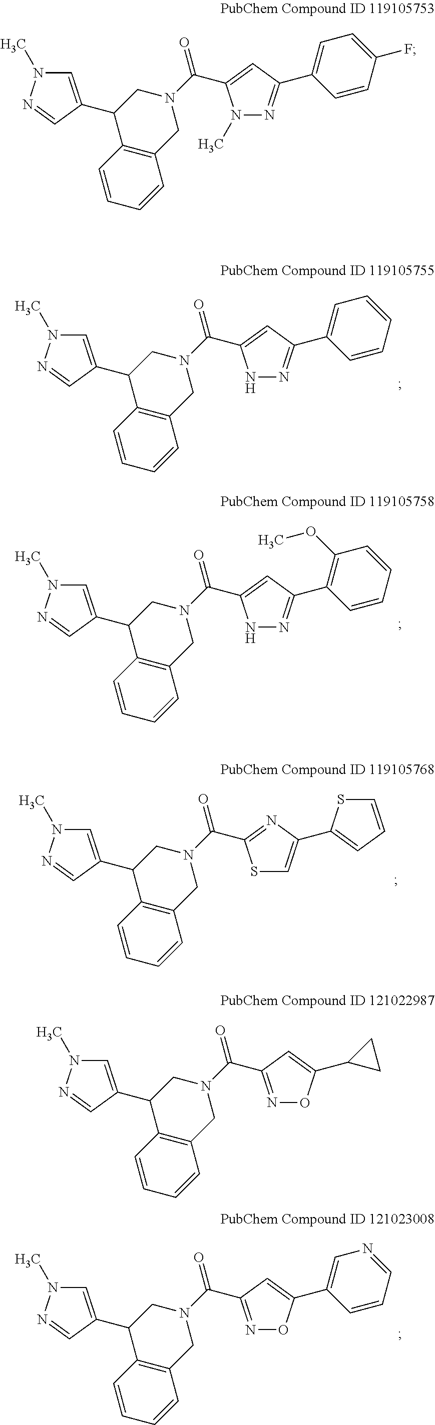 [SMILES: Cn1cc(C2CN(C(=O)c3cc(-c4ccc(F)cc4)nn3C)Cc3ccccc32)cn1 ; Cn1cc(C2CN(C(=O)c3cc(-c4ccccc4)n[nH]3)Cc3ccccc32)cn1 ; COc1ccccc1-c1cc(C(=O)N2Cc3ccccc3C(c3cnn(C)c3)C2)[nH]n1 ; Cn1cc(C2CN(C(=O)c3nc(-c4cccs4)cs3)Cc3ccccc32)cn1 ; Cn1cc(C2CN(C(=O)c3cc(C4CC4)on3)Cc3ccccc32)cn1 ; Cn1cc(C2CN(C(=O)c3cc(-c4cccnc4)on3)Cc3ccccc32)cn1] 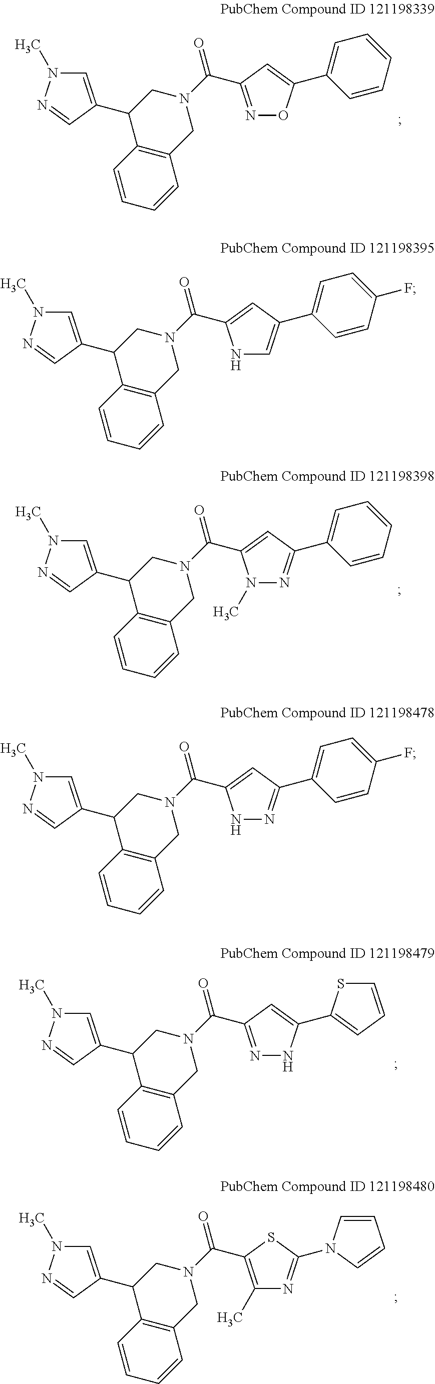 [SMILES: Cn1cc(C2CN(C(=O)c3cc(-c4ccccc4)on3)Cc3ccccc32)cn1 ; Cn1cc(C2CN(C(=O)c3cc(-c4ccc(F)cc4)c[nH]3)Cc3ccccc32)cn1 ; Cn1cc(C2CN(C(=O)c3cc(-c4ccccc4)nn3C)Cc3ccccc32)cn1 ; Cn1cc(C2CN(C(=O)c3cc(-c4ccc(F)cc4)n[nH]3)Cc3ccccc32)cn1 ; Cn1cc(C2CN(C(=O)c3cc(-c4cccs4)[nH]n3)Cc3ccccc32)cn1 ; Cc1nc(-n2cccc2)sc1C(=O)N1Cc2ccccc2C(c2cnn(C)c2)C1] 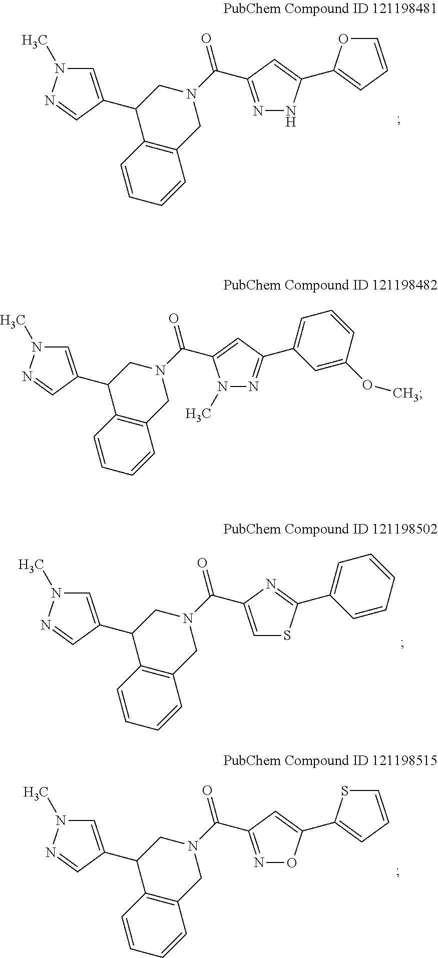 [SMILES: Cn1cc(C2CN(C(=O)c3cc(-c4ccco4)[nH]n3)Cc3ccccc32)cn1 ; COc1cccc(-c2cc(C(=O)N3Cc4ccccc4C(c4cnn(C)c4)C3)n(C)n2)c1 ; Cn1cc(C2CN(C(=O)c3csc(-c4ccccc4)n3)Cc3ccccc32)cn1 ; Cn1cc(C2CN(C(=O)c3cc(-c4cccs4)on3)Cc3ccccc32)cn1] 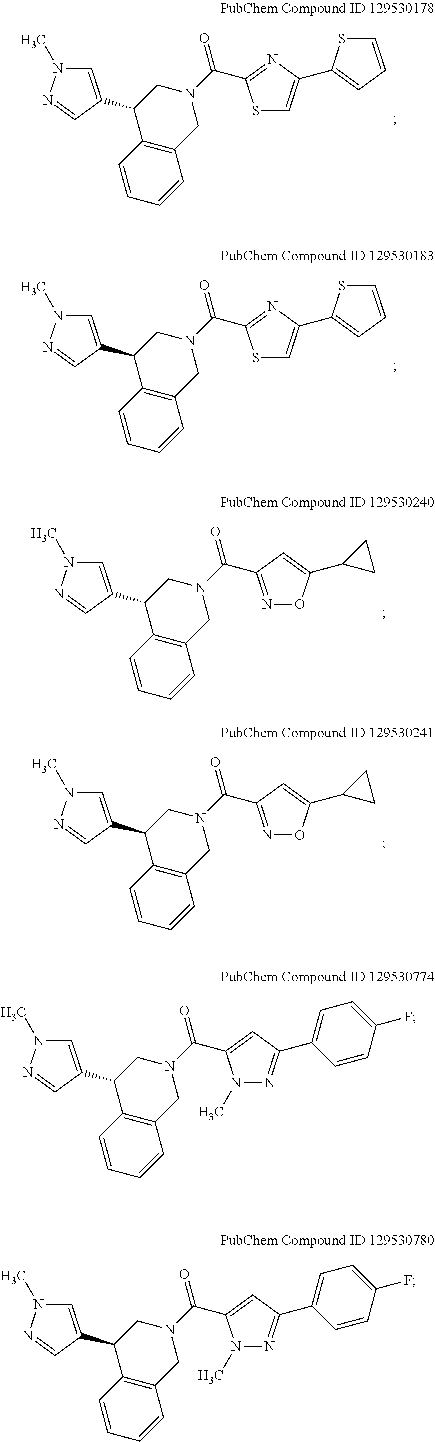 [SMILES: Cn1cc([C@@H]2CN(C(=O)c3nc(-c4cccs4)cs3)Cc3ccccc32)cn1 ; Cn1cc([C@H]2CN(C(=O)c3nc(-c4cccs4)cs3)Cc3ccccc32)cn1 ; Cn1cc([C@@H]2CN(C(=O)c3cc(C4CC4)on3)Cc3ccccc32)cn1 ; Cn1cc([C@H]2CN(C(=O)c3cc(C4CC4)on3)Cc3ccccc32)cn1 ; Cn1cc([C@@H]2CN(C(=O)c3cc(-c4ccc(F)cc4)nn3C)Cc3ccccc32)cn1 ; Cn1cc([C@H]2CN(C(=O)c3cc(-c4ccc(F)cc4)nn3C)Cc3ccccc32)cn1] 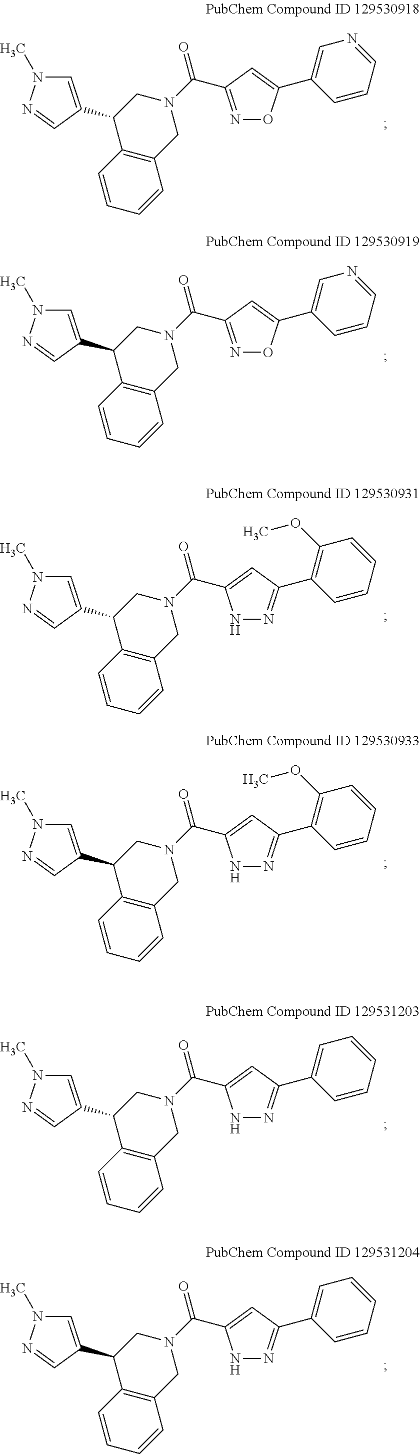 [SMILES: Cn1cc([C@@H]2CN(C(=O)c3cc(-c4cccnc4)on3)Cc3ccccc32)cn1 ; Cn1cc([C@H]2CN(C(=O)c3cc(-c4cccnc4)on3)Cc3ccccc32)cn1 ; COc1ccccc1-c1cc(C(=O)N2Cc3ccccc3[C@H](c3cnn(C)c3)C2)[nH]n1 ; COc1ccccc1-c1cc(C(=O)N2Cc3ccccc3[C@@H](c3cnn(C)c3)C2)[nH]n1 ; Cn1cc([C@@H]2CN(C(=O)c3cc(-c4ccccc4)n[nH]3)Cc3ccccc32)cn1 ; Cn1cc([C@H]2CN(C(=O)c3cc(-c4ccccc4)n[nH]3)Cc3ccccc32)cn1]
or an agrochemically acceptable salt, stereoisomer, enantiomer, tautomer or N-oxide thereof.

2. The compound of formula (I) according to claim 1, wherein B¹ and B² are CH.

3. The compound of formula (I) according to claim 1, wherein R¹ is selected from hydrogen or methyl, and wherein R² and R³ are hydrogen.

4. The compound of formula (I) according to claim 1, wherein R⁴ is selected from hydrogen, or C₁-C₄ alkyl.

5. The compound of formula (I) according to claim 1, wherein R⁵ and R⁶ are hydrogen.

6. The compound of formula (I) according to claim 1, wherein Q is selected from 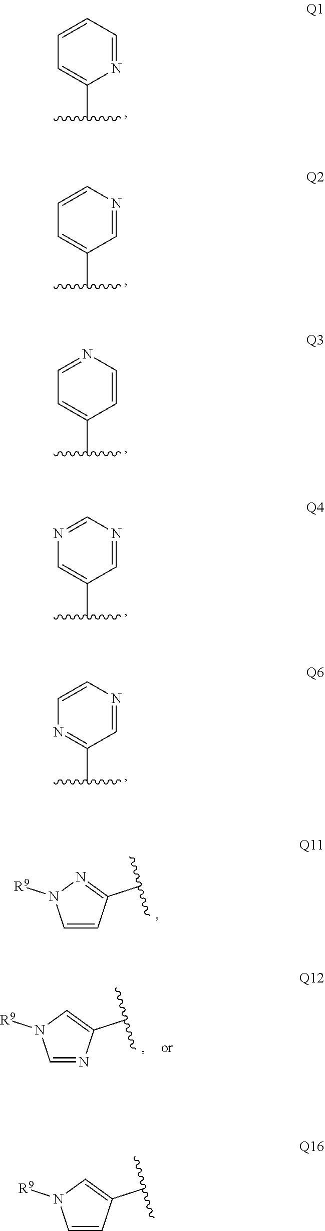
wherein any of said Q groups is unsubstituted or substituted with 1 or 2 substituents selected from fluorine, chlorine, bromine, cyano or methyl, and wherein R⁹ is methyl, and
wherein [symbol] denotes the position, which is attached to the tetrahydroisoquinoline-moiety.

7. The compound of formula (I) according to claim 6, wherein Q is selected from 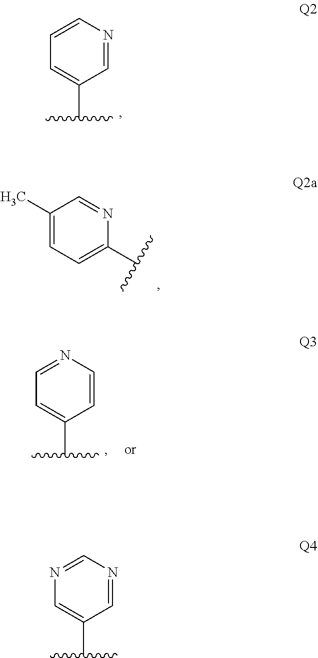
wherein [symbol] denotes the position, which is attached to the tetrahydroisoquinoline-moiety.

8. The compound of formula (I) according to claim 1, wherein Z¹ is selected from C₁-C₄ alkyl, phenyl, a 5- to 6-membered heteroaryl, or C₃-C₆-cycloalkyl; wherein any of said 5- or 6-membered heteroaryl contains 1 heteroatom selected from N, and wherein any of said phenyl and 5- to 6-membered heteroaryl are unsubstituted or substituted with 1 or 2 substituents independently selected from fluoro, chloro, methyl, ethyl, ethynyl, methoxy, or methylsulfonyl; and wherein said C₃-C₆-cycloalkyl is unsubstituted or substituted with 1 or 2 substituents selected from methyl.

9. The compound of formula (I) according to claim 8, wherein Z¹ is 2,4-difluorophenyl, 3,5-difluoro-2-pyridyl, 5-fluoro-2-pyridyl, 3-fluoro-2-pyridyl, 3,5-difluoro-2-furyl, 3-fluoro-2-furyl, 5-fluoro-2-furyl, 3,5-difluoro-2-thienyl, 3-fluoro-2-thienyl, 5-fluoro-2-thienyl, 2-fluorophenyl, 4-fluorophenyl, or phenyl.

10. A compound of formula (I) according to claim 1, wherein the compound of formula (I) is a compound of formula (I-A1), 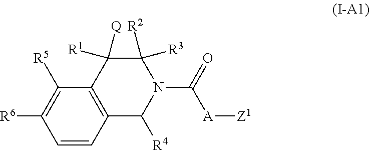
wherein R¹, R², R³, R⁴, R⁵, Q and Z¹ are as defined for the compounds of formula (I) according to claim 1, and A is selected from 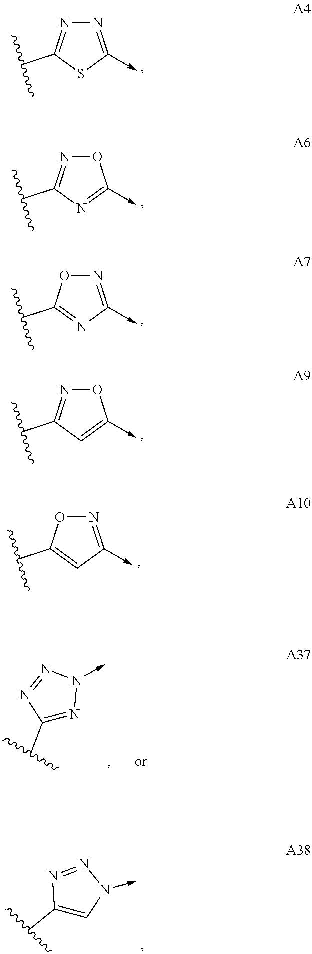
wherein [symbol] denotes the position, which is attached to the C(═O) group and the arrow denotes the position, which is attached to the Z¹ group.

11. The compound of formula (I-A1) according to claim 10, wherein A is selected from: 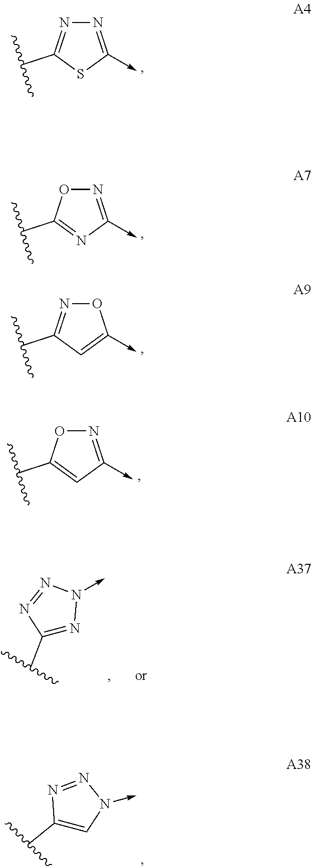
wherein [symbol] denotes the position, which is attached to the C(═O) group and the arrow denotes the position, which is attached to the Z¹ group.

12. Use of a compound of formula (I) as a fungicide 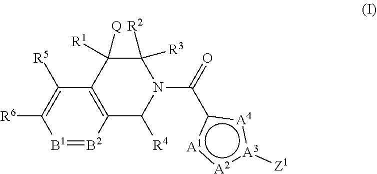
wherein
Q is selected from a 5- or 6-membered heteroaryl, wherein any of said 5- or 6-membered heteroaryl contains 1, 2, 3 or 4 heteroatoms independently selected from N, O or S, with the proviso that no more than one is O or S; and wherein any of said 5- or 6-membered heteroaryl is unsubstituted or substituted with 1, 2 or 3 substituents independently selected from halogen, cyano, C₁-C₄-alkyl, C₁-C₄-haloalkyl, or C₃-C₆cycloalkyl;
R¹ is selected from hydrogen, halogen, C₁-C₄haloalkyl, C₃-C₆ cycloalkyl, or C₁-C₄ alkyl;
R² and R³ are independently selected from hydrogen, or C₁-C₄-alkyl;
R⁴ is selected from hydrogen, C₁-C₄-alkyl, C₁-C₄-alkylcarbonyl, N—C₁-C₄-alkoxy-C—C₁-C₄ alkyl-carbonimidoyl, N-hydroxy-C—C₁-C₄-alkyl-carbonimidoyl, C₁-C₄-alkoxycarbonyl, N-methoxy-N-methyl-carbonyl, C₁-C₄ alkylaminocarbonyl, di(C₁-C₄alkyl)aminocarbonyl, phenyl, a 5- to 6-membered heteroaryl, or C₃-C₆-cycloalkyl;
wherein any of said 5- or 6-membered heteroaryl contains 1, 2, 3 or 4 heteroatoms independently selected from N, O or S, with the proviso that no more than one is O or S;
wherein said phenyl and 5- to 6-membered heteroaryl are unsubstituted or substituted with 1, 2 or 3 substituents independently selected from halogen, cyano, C₁-C₄-alkyl, C₁-C₄-haloalkyl, carboxy, or C₁-C₄-alkoxy; and wherein said C₃-C₆-cycloalkyl is unsubstituted or substituted with 1, 2, or 3 substituents independently selected from halogen, cyano, C₁-C₄alkyl, C₁-C₄haloalkyl, or C₁-C₄alkoxy;
B¹ is selected from CR⁷, or N;
B² is selected from CR⁸, or N;
R⁵, R⁶, R⁷ and R⁸ are independently selected from hydrogen, halogen, C₁-C₄ alkyl, C₁-C₄ haloalkyl, C₁-C₄ alkoxy, C₁-C₄haloalkoxy, C₂-C₄alkenyloxy, C₂-C₄ alkynyloxy, C₁-C₄ alkylsulfanyl, C₁-C₄ alkylsulfinyl, C₁-C₄ alkylsulfonyl, C₁-C₄ alkoxy-C₁-C₄ alkyl, N—C₁-C₄alkylamino, N,N-diC₁-C₄alkylamino, C₁-C₄ alkoxycarbonyl, C₁-C₄ alkylcarbonyl, N—C₁-C₄ alkoxy-C₁-C₄ alkyl-carbonimidoyl, N-hydroxy-C₁-C₄ alkyl-carbonimidoyl, hydroxy, trifluoromethylsulfonyloxy, cyano, carboxy, amino, phenyl, 5- or 6-membered heteroaryl, or C₃-C₆ cycloalkyl; wherein any of said 5- or 6-membered heteroaryl contains 1, 2, 3 or 4 heteroatoms independently selected from N, O or S, with the proviso that no more than one is O or S; and wherein any of said phenyl, 5- or 6-membered heteroaryl and C₃-C₆-cycloalkyl are unsubstituted or substituted with 1, 2 or 3 substituents independently selected from halogen, cyano, C₁-C₄ alkyl, C₁-C₄haloalkyl, or C₁-C₄alkoxy;
A¹, A², A⁴ are independently selected from of methine, N, NR¹⁰, O, or S, with the proviso that at least one of A¹, A² and A⁴ is selected from N, NR¹⁰, O or S, and that no more than one of A¹, A² and A⁴ is O or S, wherein R¹⁰ is selected from hydrogen or C₁-C₄ alkyl;
A³ is C or N with the proviso that when A³ is N, A¹, A² and A⁴ are methine, N or NR¹⁰ wherein R¹⁰ is selected from hydrogen or C₁-C₄ alkyl; and
Z¹ is selected from C₁-C₄ alkyl, phenyl, 5- or 6-membered heteroaryl, or C₃-C₆ cycloalkyl, wherein any of said 5- or 6-membered heteroaryl contains 1, 2, 3, or 4 heteroatoms independently selected from N, O, or S, with the proviso that no more than one is O or S; wherein any of said phenyl, and 5- or 6-membered heteroaryl are unsubstituted or substituted by 1, 2, or 3 substituents independently selected from halogen, cyano, C₁-C₄ alkyl, C₂-C₄ alkenyl, C₁-C₄ haloalkyl, C₁-C₄ alkoxy, C₁-C₄ haloalkoxy, C₁-C₄ alkylsulfanyl, C₁-C₄ alkylsulfinyl, C₁-C₄ alkylsulfonyl, or C₂-C₄ alkynyl; and wherein said C₃-C₆ cycloalkyl is unsubstituted or substituted by 1, 2, or 3 substituents independently selected from halogen, cyano, C₁-C₄ alkyl, C₁-C₄ haloalkyl, or C₁-C₄alkoxy;
or an agrochemically acceptable salt, stereoisomer, or N-oxide thereof.

13. An agrochemical composition comprising a fungicidally effective amount of a compound of formula (I) as defined in claim 1.

14. The agrochemical composition according to claim 13, further comprising at least one additional active ingredient and/or an agrochemically-acceptable diluent or carrier.

15. A method of controlling or preventing infestation of useful plants by phytopathogenic microorganisms, wherein a fungicidally effective amount of a compound of formula (I) as defined in claim 1, or a composition comprising the compound of formula (I), is applied to the plants, to parts thereof or the locus thereof.
